SEQUENCE LISTING (54 sequences)

SEQ ID NO: 1 - protein, 9 aa, Artificial Sequence
  homing peptide
CRGDKGPDC

SEQ ID NO: 2 - protein, 10 aa, Artificial Sequence
  homing peptide
CCRGDKGPDC

SEQ ID NO: 3 - protein, 16 aa, Artificial Sequence
  homing peptide
AKPAPPKPEPKPKKAP

SEQ ID NO: 4 - protein, 15 aa, Artificial Sequence
  homing peptide
AKVKDEPQRRSARLS

SEQ ID NO: 5 - protein, 7 aa, Artificial Sequence
  homing peptide
CAGALCY

SEQ ID NO: 6 - protein, 9 aa, Artificial Sequence
  homing peptide
CAGRRSAYC

SEQ ID NO: 7 - protein, 9 aa, Artificial Sequence
  homing peptide
CARSKNKDC

SEQ ID NO: 8 - protein, 9 aa, Artificial Sequence
  homing peptide
CDCRGDCFC

SEQ ID NO: 9 - protein, 5 aa, Artificial Sequence
  homing peptide
CDTRL

SEQ ID NO: 10 - protein, 5 aa, Artificial Sequence
  homing peptide
CGKRK

SEQ ID NO: 11 - protein, 10 aa, Artificial Sequence
  homing peptide
CGLIIQKNEC

SEQ ID NO: 12 - protein, 7 aa, Artificial Sequence
  homing peptide
CGNKRTR

SEQ ID NO: 13 - protein, 9 aa, Artificial Sequence
  homing peptide
CGNKRTRGC

SEQ ID NO: 14 - protein, 9 aa, Artificial Sequence
  homing peptide
CGRRAGGSC

SEQ ID NO: 15 - protein, 7 aa, Artificial Sequence
  homing peptide
CKAAKNK

SEQ ID NO: 16 - protein, 11 aa, Artificial Sequence
  homing peptide
CKGGRAKDCGG

SEQ ID NO: 17 - protein, 9 aa, Artificial Sequence
  homing peptide
CLSDGKRKC

SEQ ID NO: 18 - protein, 12 aa, Artificial Sequence
  homing peptide
CMYIEALDKYAC

SEQ ID NO: 19 - protein, 12 aa, Artificial Sequence
  homing peptide
KKCGGGGIRLRG

SEQ ID NO: 20 - protein, 10 aa, Artificial Sequence
  homing peptide
CNAGESSKNC

SEQ ID NO: 21 - protein, 5 aa, Artificial Sequence
  homing peptide
CNGRC

SEQ ID NO: 22 - protein, 9 aa, Artificial Sequence
  homing peptide
CNRRTKAGC

SEQ ID NO: 23 - protein, 9 aa, Artificial Sequence
  homing peptide
CPGPEGAGC

SEQ ID NO: 24 - protein, 9 aa, Artificial Sequence
  homing peptide
CPKTRRPVC

SEQ ID NO: 25 - protein, 9 aa, Artificial Sequence
  homing peptide
CPRECESIC

SEQ ID NO: 26 - protein, 9 aa, Artificial Sequence
  homing peptide
CRAKSKVAC

SEQ ID NO: 27 - protein, 9 aa, Artificial Sequence
  homing peptide
CREAGRKAC

SEQ ID NO: 28 - protein, 5 aa, Artificial Sequence
  homing peptide
CREKA

SEQ ID NO: 29 - protein, 9 aa, Artificial Sequence
  homing peptide
CRGDKGPDC

SEQ ID NO: 30 - protein, 7 aa, Artificial Sequence
  homing peptide
CRGRRST

SEQ ID NO: 31 - protein, 6 aa, Artificial Sequence
  homing peptide
CRKDKC

SEQ ID NO: 32 - protein, 5 aa, Artificial Sequence
  homing peptide
CRPPR

SEQ ID NO: 33 - protein, 9 aa, Artificial Sequence
  homing peptide
CRRETAWAC

SEQ ID NO: 34 - protein, 6 aa, Artificial Sequence
  homing peptide
CRSRKG

SEQ ID NO: 35 - protein, 9 aa, Artificial Sequence
  homing peptide
CSRPRRSEC

SEQ ID NO: 36 - protein, 10 aa, Artificial Sequence
  homing peptide
CTTHWGFTLC

SEQ ID NO: 37 - protein, 9 aa, Artificial Sequence
  homing peptide
CVPELGHEC

SEQ ID NO: 38 - protein, 13 aa, Artificial Sequence
  homing peptide
EKGEGALPTGKSK

SEQ ID NO: 39 - protein, 6 aa, Artificial Sequence
  homing peptide
FALGEA

SEQ ID NO: 40 - protein, 13 aa, Artificial Sequence
  homing peptide
GLNGLSSADPSSD

SEQ ID NO: 41 - protein, 8 aa, Artificial Sequence
  homing peptide
GSMSIARL

SEQ ID NO: 42 - protein, 8 aa, Artificial Sequence
  homing peptide
GVSFLEYR

SEQ ID NO: 43 - protein, 7 aa, Artificial Sequence
  homing peptide
IFLLWQR

SEQ ID NO: 44 - protein, 10 aa, Artificial Sequence
  homing peptide
IFLLWQRCRR

SEQ ID NO: 45 - protein, 5 aa, Artificial Sequence
  homing peptide
PEPHC

SEQ ID NO: 46 - protein, 13 aa, Artificial Sequence
  homing peptide
PISNDQKVSDDDK

SEQ ID NO: 47 - protein, 15 aa, Artificial Sequence
  homing peptide
RMWPSSTVNLSAGRR

SEQ ID NO: 48 - protein, 7 aa, Artificial Sequence
  homing peptide
RPARPAR

SEQ ID NO: 49 - protein, 7 aa, Artificial Sequence
  homing peptide
SMSIARL

SEQ ID NO: 50 - protein, 13 aa, Artificial Sequence
  homing peptide
VDEDRASLLKSQE

SEQ ID NO: 51 - protein, 7 aa, Artificial Sequence
  homing peptide
VSFLEYR

SEQ ID NO: 52 - protein, 11 aa, Artificial Sequence
  homing peptide
WNAPAEEWGNW

SEQ ID NO: 53 - protein, 6 aa, Artificial Sequence
  peptidyl linker sequence
PLGLWA

SEQ ID NO: 54 - protein, 4 aa, Artificial Sequence
  peptidyl linker sequence
GFLG

(19) United States
(12) Patent Application Publication
(10) Pub. No.: US 2019/0365897 A1
(43) Pub. Date: Dec. 5, 2019

(54) THERAPEUTIC COMPOSITIONS AND RELATED METHODS FOR PHOTOIMMUNOTHERAPY

(71) Applicant: Rakuten Medical, Inc.

(72) Inventors: Miguel GARCIA-GUZMAN, San Diego, CA (US); Lewis R. MAKINGS, Encinitas, CA (US); Eileen Sun CHIN, San Diego, CA (US)

(73) Assignees: Rakuten Medical, Inc., San Mateo, CA (US); Rakuten Medical, Inc., San Mateo, CA (US)

(21) Appl. No.: 16/487,419

(22) PCT Filed: Feb. 22, 2018
(86) PCT No.: PCT/US18/19294

§ 371 Date: Aug. 20, 2019

Related U.S. Application Data
(60) Provisional application No. 62/462,898, filed on Feb. 23, 2017.

(51) Int. Cl. A61K 41/00; A61K 47/68; A61P 35/00

(52) U.S. Cl. CPC A61K 41/0071; A61K 47/6845; A61K 45/06; A61P 35/00; A61K 47/6889

(57) ABSTRACT

Provided are conjugates, e.g., dual conjugates, compositions and methods for use in photoimmunotherapy, such as photoimmunotherapy induced by activation of a phthalocyanine dye in the dual conjugate. In some embodiments, the dual conjugate contains a targeting molecule and a therapeutic agent. In some embodiments, the phthalocyanine-dye in the conjugate, e.g., dual conjugate, can be activated by irradiation with near-infrared light. Also provided are therapeutic methods using the conjugates, e.g., dual conjugates, and compositions for treatment of a lesion associated with diseases and conditions, including tumors or cancers. Features of the conjugates, e.g., dual conjugates, compositions, combinations and methods, including the dose of the conjugate, provide various advantages, such as efficient delivery and targeting of the therapeutic agent to the site of the lesion.

### CROSS-REFERENCE TO RELATED APPLICATIONS

This application claims priority from U.S. provisional application No. 62/462,898, filed Feb. 23, 2017, entitled “THERAPEUTIC COMPOSITIONS AND RELATED METHODS FOR PHOTOIMMUNOTHERAPY,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 751702000640SeqList.TXT, created Feb. 22, 2018 which is 10,121 bytes in size. The information in the electronic format of the Sequence Listing is incorporated by reference in its entirety.

### FIELD

The present disclosure relates to conjugates, e.g., dual conjugates, compositions and methods for use in photoimmunotherapy, such as photoimmunotherapy induced by activation of a phthalocyanine dye in the dual conjugate. In some embodiments, the dual conjugate contains a targeting molecule and a therapeutic agent. In some embodiments, the phthalocyanine-dye in the conjugate, e.g., dual conjugate, can be activated by irradiation with near-infrared light. The disclosure also provides therapeutic methods using the conjugates, e.g., dual conjugates, and compositions for treatment of a lesion associated with diseases and conditions, including tumors or cancers. Features of the conjugates, e.g., dual conjugates, compositions, combinations and methods, including the dose of the conjugate, provide various advantages, such as efficient delivery and targeting of the therapeutic agent to the site of the lesion.

### BACKGROUND

Various therapies are available for treating disease, such as cancer. For example, photoimmunotherapy (PIT) is a method that uses a photosensitizer conjugated to an antibody or other targeting molecule to target a cell surface molecule in order to permit the targeted killing of specific cells. In some cases, PIT can selectively target disease cells, such as tumor cells, and thereby selectively kill such cells without damaging healthy cells. Improved strategies are needed for photoimmunotherapy methods, for example, to increase the efficacy of treatment. Provided are conjugates, compositions and methods that meet such needs.

### SUMMARY

Provided herein in some embodiments is a dual conjugate including a phthalocyanine dye, a targeting molecule, and a therapeutic agent. In some embodiments, the phthalocyanine dye and therapeutic agent are each independently linked to the targeting molecule. In some embodiments, the targeting molecule and therapeutic agent are each independently linked to the phythalocyanine dye. In some embodiments, the phythalocyanine dye and the targeting molecule are each independently linked to the therapeutic agent.

In some embodiments, the dual conjugate includes the following components: (phthalocyanine dye)n, (targeting molecule)q and (therapeutic agent)m, wherein n, q and m, which are selected independently, are at least 1. In some embodiments, n and q, which are selected independently, are 1 to 5. In some embodiments, n and m, which are selected independently, are 1 to 5. In some embodiments, q is 1, n is between 1 and 100, and m is between 1 and 5. In some embodiments, the ratio of n to q is from or from about 1 to about 1000, from or from about 1 to about 10 or from or from about 2 to about 5.

In some embodiments, the targeting molecule is capable of binding a cell surface molecule on a cell in a microenvironment of a lesion. In some embodiments, the targeting molecule is linked directly with the phthalocyanine dye or the therapeutic agent. In some embodiments, the linkage between the targeting molecule and the phthalocyanine dye and/or the therapeutic agent is covalent or non-covalent. In some embodiments, the phthalocyanine dye is linked directly with the targeting molecule or the therapeutic agent. In some embodiments, the linkage between the phthalocyanine dye and the targeting molecule and/or the therapeutic agent is covalent or non-covalent. In some embodiments, the therapeutic agent is linked directly with the phthalocyanine dye or the targeting molecule. In some embodiments, the linkage between the therapeutic agent and the phthalocyanine dye or the targeting molecule is covalent or non-covalent.

In some embodiments, therapeutic agent is linked indirectly via a linker to the phthalocyanine dye or the targeting molecule. In some embodiments, the targeting molecule is linked indirectly via a linker to the phthalocyanine dye or the therapeutic agent. In some embodiments, the phthalocyanine dye is linked indirectly via a linker to the targeting molecule or the therapeutic agent.

In some embodiments, the linker is a peptide or a polypeptide or is a chemical linker. In some embodiments, the linker is a releasable linker or a cleavable linker. In some embodiments, the releasable linker or the cleavable linker is released or cleaved in the microenvironment of the lesion. In some embodiments, the lesion is a tumor, and the releasable linker or the cleavable linker is released or cleaved in the tumor microenvironment (TME). In some embodiments, the releasable linker or the cleavable linker is released or cleaved by a matrix metalloproteinase (MMP) present in in the TME. In some embodiments, the cleavable linker contains the sequence of amino acids set forth in PLGLWA.

In some embodiments, the releasable linker or the cleavable linker is released or cleaved in hypoxic conditions or acidic conditions. In some embodiments, the cleavable linker is cleavable under acidic conditions, and the cleavable linker includes one or more hydrazone, semicarbazone, thiosemicarbazone, cis-aconitic amide, orthoester, acetal, ketal or thioether linkages. In some embodiments, the cleavable linker is cleavable under hypoxic conditions, and the linker includes one or more disulfide linkages. In some embodiments, the cleavable linker is cleavable by light irradiation, and the linker includes one or more photolabile phenacyl ester, photolabile hydrazine or photolabile o-nitrobenzyl linkages or photolabile quinoxaline with thioether.

In some embodiments, the therapeutic agent is an immune modulating agent and/or an anti-cancer agent. In some embodiments, the immune modulating agent is a cytokine or is an agent that induces increased expression of a cytokine in the microenvironment of the lesion. In some embodiments, the cytokine is selected from among IL-1, IL-1α, IL-2, IL-3, IL-4, IL-5, IL-6, IL-7, IL-8, IL-9, IL-10, IL-11, IL-12, IL-15, interferon (IFN)-α, IFN-β, IFN-γ, tumor necrosis factor (TNF)-α, TNF-β, human growth hormone, N-methionyl human growth hormone, parathyroid hormone, thyroxine, insulin, proinsulin, relaxin, prorelaxin, glycoprotein hormones such as follicle stimulating hormone (FSH), thyroid stimulating hormone (TSH), and luteinizing hormone (LH), hepatic growth factor, fibroblast growth factor (FGF), prolactin, placental lactogen, tumor necrosis factor-α and -β, mullerian-inhibiting substance, mouse gonadotropin-associated peptide, inhibin, activin, vascular endothelial growth factor (VEGF), integrin, thrombopoietin (TPO), nerve growth factors (NGF)-β, platelet-growth factor, transforming growth factor (TGF)-α, TGF-β, insulin-like growth factor (IGF)-1, IGF-2, erythropoietin (EPO), osteoinductive factors, macrophage-CSF (M-CSF), granulocyte-macrophage-CSF (GM-CSF), granulocyte-CSF (G-CSF), leukemia inhibitory factor (LIF), kit ligand (KL) and/or a portion and/or combination thereof In some embodiments, the immune modulating agent is a cytokine and the cytokine is IL-2, IL-4, IL-12, IFN-γ, TNF-α or GM-CSF.

In some embodiments, the immune modulating agent is an immune checkpoint inhibitor. In some embodiments, the immune modulating agent specifically binds a molecule selected from among CD25, PD-1, PD-L1, PD-L2, CTLA-4, LAG-3, TIM-3, 4-1BB, GITR, CD40, CD40L, OX40, OX40L, CXCR2, B7-H3, B7-H4, BTLA, HVEM, CD28 VISTA, ICOS, ICOS-L, CD27, CD30, STING, and A2A adenosine receptor. In some embodiments, the immune modulating agent is an antibody or an antigen-binding fragment thereof, a small molecule or a polypeptide. In some embodiments, the immune modulating agent is selected from among nivolumab, pembrolizumab, pidilizumab, MK-3475, BMS-936559, MPDL3280A, ipilimumab, tremelimumab, IMP31, BMS-986016, urelumab, TRX518, dacetuzumab, lucatumumab, SEQ-CD40, CP-870, CP-893, MED16469, MED14736, MOXR0916, AMP-224, and MSB001078C, or is an antigen-binding fragment thereof.

In some embodiments, the anti-cancer agent is an alkylating agent, a platinum drug, an antimetabolite, an anti-tumor antibiotic, a topoisomerase inhibitor, a mitotic inhibitor, a corticosteroid, a proteasome inhibitor, a kinase inhibitor, a histone-deacetylase inhibitor, an anti-neoplastic agent, or a combination thereof.

In some embodiments, the anti-cancer agent is an antibody or an antigen-binding fragment thereof, a small molecule or a polypeptide. In some embodiments, the anti-cancer agent is selected from among 5-Fluorouracil/leukovorin, oxaliplatin, irinotecan, regorafenib, ziv-afibercept, capecitabine, cisplatin, paclitaxel, toptecan, carboplatin, gemcitabine, docetaxel, 5-FU, ifosfamide, mitomycin, pemetrexed, vinorelbine, carmustine wager, temozolomide, methotrexate, capacitabine, lapatinib, etoposide, dabrafenib, vemurafenib, liposomal cytarabine, cytarabine, interferon alpha, erlotinib, vincristine, cyclophosphamide, lomusine, procarbazine, sunitinib, somastostatin, doxorubicin, pegylated liposomal encapsulated doxorubicin, epirubicin, eribulin, albumin-bound paclitaxel, ixabepilone, cotrimoxazole, taxane, vinblastine, temsirolimus, temozolomide, bendamustine, oral etoposide, everolimus, octreotide, lanredtide, dacarbazine, mesna, pazopanib, eribulin, imatinib, regorafenib, sorafenib, nilotinib, dasantinib, celecoxib, tamoxifen, toremifene, dactinomycin, sirolimus, crizotinib, certinib, enzalutamide, abiraterone acetate, mitoxantrone, cabazitaxel, fluoropyrimidine, oxaliplatin, leucovorin, afatinib, ceritinib, gefitinib, cabozantinib, oxoliplatin and auroropyrimidine.

In some embodiments, the anti-cancer agent is selected from among bevacizumab, cetuximab, panitumumab, ramucirumab, ipilimumab, rituximab, trastuzumab, ado-trastuzumab emtansine, pertuzumab, nivolumab, lapatinib, dabrafenib, vemurafenib, erlotinib, sunitinib, pazopanib, imatinib, regorafenib, sorafenib, nilotinib, dasantinib, celecoxib, crizotinib, certinib, afatinib, axitinib, bevacizumab, bosutinib, cabozantinib, afatinib, gefitinib, temsirolimus, everolimus, sirolimus, ibrutinib, imatinib, lenvatinib, olaparib, palbociclib, ruxolitinib, trametinib, vandetanib or vismodegib, or an antigen-binding fragment thereof.

In some embodiments, the phthalocyanine dye has a maximum absorption wavelength from or from about 600 nm to about 850 nm. In some embodiments, the phthalocyanine dye contains the formula:

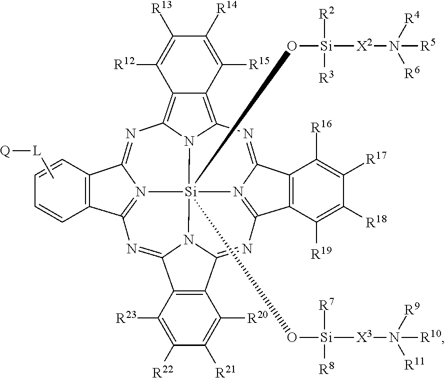

wherein:

L is a linker;

Q is a reactive group for attachment of the dye to the targeting molecule;

R², R³, R⁷, and R⁸ are each independently selected from optionally substituted alkyl and optionally substituted aryl;

R⁴, R⁵, R⁶, R⁹, R¹⁰, and R¹¹ are each independently selected from hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ contains a water soluble group;

R¹², R¹³, R¹⁴, R¹⁵, R¹⁶, R¹⁷, R¹⁸, R¹⁹, R²⁰, R²¹, R²² and R²³ are each independently selected from hydrogen, halogen, optionally substituted alkylthio, optionally substituted alkylamino and optionally substituted alkoxy; and

X² and X³ are each independently C₁-C₁₀ alkylene, optionally interrupted by a heteroatom.

In some embodiments, the phthalocyanine dye contains the formula:

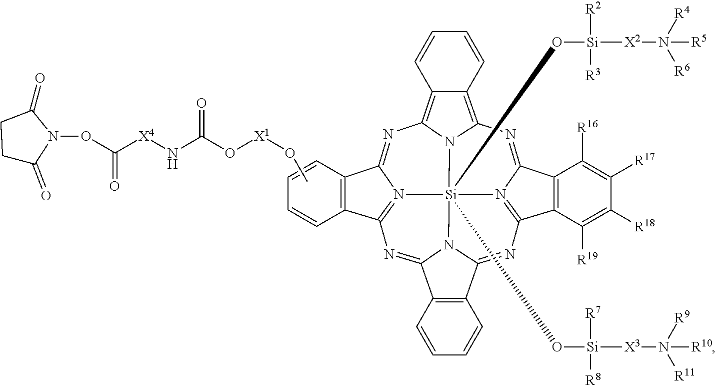

wherein:

X¹ and X⁴ are each independently a C₁-C₁₀ alkylene optionally interrupted by a heteroatom;

R², R³, R⁷, and R⁸ are each independently selected from optionally substituted alkyl and optionally substituted aryl;

R⁴, R⁵, R⁶, R⁹, R¹⁰, and R¹¹ are each independently selected from hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ contains a water soluble group; and

R¹⁶, R¹⁷, R¹⁸ and R¹⁹ are each independently selected from hydrogen, halogen, optionally substituted alkylthio, optionally substituted alkylamino and optionally substituted alkoxy.

In some embodiments, the phthalocyanine dye includes IRDye 700DX (IR700).

In some embodiments, the targeting molecule is an antibody or an antigen-binding fragment thereof. In some embodiments, the antibody is an antigen-binding fragment that is a Fab, single V_{H} domain, a single chain variable fragment (scFv), a multivalent scFv, a bispecific scFv or an scFv-CH₃ dimer.

In some embodiments, the lesion is premalignant dysplasia, carcinoma in situ, neoplasm, hyperplasia tumor or a tumor that is associated with a cancer.

Also provided herein in some embodiments is a composition containing any of the dual conjugates described herein. In some embodiments, the composition further includes a pharmaceutically acceptable excipient.

Also provided herein in some embodiments is a kit that contains any of the dual conjugates or compositions described herein and optionally instructions for use.

Also provided herein in some embodiments is a method of treating a lesion in a subject including administering to the subject a therapeutically effective amount of the dual conjugate of any of claims 1-43 or the composition of claim 44 or claim 45 or the kit of claim 46; and after administering the conjugate, irradiating the lesion at a wavelengths to induce phototoxic activity of the conjugate.

In some embodiments, the lesion is carried out at a wavelength of 500 nm to 900 nm, inclusive, at a dose of at least 1 J cm⁻or 1 J/cm of fiber length. In some embodiments, irradiating of the lesion is carried out at wavelength of 600 nm to 850 nm. In some embodiments, irradiating of the lesion is carried out at a wavelength of 690±50 nm or at a wavelength of or about 690±20 nm. In some embodiments, irradiating of the lesion is carried out at a dose of from or from about 2 J cm⁻² to about 400 J cm⁻² or from or from about 2 J/cm fiber length to about 500 J/cm fiber length.

In some embodiments, irradiating of the lesion is carried out at a dose of at least or at least about 2 J cm², 5 J cm⁻², 10 J cm⁻², 25 J cm⁻², 50 J cm⁻², 75 J cm⁻², 100 J cm⁻², 150 J cm⁻², 200 J cm⁻², 300 J cm⁻², 400 J cm⁻², or 500 J cm⁻²; or irradiating of the lesion is carried out at a dose of at least or at least about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

In some embodiments, the lesion is a tumor or a tumor that is associated with a cancer. In some embodiments, the tumor is a sarcoma or carcinoma. In some embodiments, the tumor is a carcinoma that is a squamous cell carcinoma, basal cell carcinoma or adenocarcinoma. In some embodiments, the tumor is a carcinoma that is a carcinoma of the bladder, pancreas, colon, ovary, lung, breast, stomach, prostate, cervix, esophagus or head and neck. In some embodiments, the cancer is a cancer located at the head and neck, breast, liver, colon, ovary, prostate, pancreas, brain, cervix, bone, skin, eye, bladder, stomach, esophagus, peritoneum, or lung.

In some embodiments, irradiating of the lesion is carried out between or between about 30 minutes and about 96 hours after administering the method.

In some embodiments, the dual conjugate is administered at a dose from or from about 50 mg/m² to about 5000 mg/m², from about 250 mg/m² to about 2500 mg/m², from about 750 mg/m² to about 1250 mg/m² or from about 100 mg/m² to about 1000 mg/m².

In some embodiments, the method further includes administering an additional therapeutic agent or anti-cancer treatment. In some embodiments, the dual conjugate is combined with another therapeutic for the treatment of the lesion, disease, or condition. In some embodiments, the additional anti-cancer treatment includes radiation therapy.

In some embodiments, the lesion targeted comprises neurons and the disease, disorder or condition is a neurological disorder, which optionally comprises pain. In some embodiments, the lesion targeted comprises fat cells or adipocytes and the disease, disorder or condition comprises excess fat. In some embodiments, the lesion targeted comprises pathogen infected cells and the disease, disorder or condition comprises an infection. In some embodiments, the lesion targeted comprises an inflammatory cell and the disease, disorder or condition comprises inflammation.

### BRIEF DESCRIPTION OF THE DRAWINGS

FIG. 1A shows the effect of PIT treatment in A431 and FaDu cells using Cetuximab-IRDye 700DX on the amount of HMGB1 detected in extracellular solution.

FIG. 1B shows the upregulation of dendritic cell (DC) maturation markers on immature dendric cells (iDCs) co-cultured with tumors subjected to PIT via cetuximab-IRDye 700DX.

FIG. 1C shows the effect on activating antigen-presenting cells by co-culturing with PIT-treated A431 or FaDu cells (treated using Cetuximab-IRDye 700DX and in the presence of light irradiation) or with non-PIT-treated A431 or FaDu cells (treated using Cetuximab-IRDye 700DX but with no light irradiation), as assessed by the expression of the exemplary activation marker CD86 on THP-1 cells.

FIG. 2 shows the effect on activation of dendritic cells by priming dendritic cells with PIT-treated tumor cells (treated using Cetuximab-IRDye 700DX) or non-PIT treated tumor cells (treated using Cetuximab-IRDye 700DX but with no light irradiation) followed by their stimulation with an immune modulator (Poly I:C) as assessed by the expression of exemplary activation markers CD80 and CD86.

FIG. 3A shows the effect of IFNgamma treatment on the percent death of BxPC3 cells.

FIG. 3B shows the effect of IFNgamma treatment on PD-L1 expression in BxPC3 cells.

FIG. 3C shows the effect of IFNgamma treatment on anti-PD-L1 IRDye 700DX PIT killing activity in BxPC3 cells.

### DETAILED DESCRIPTION

Provided herein are conjugates, e.g., dual conjugates, containing a photosensitizer, such as a phthalocyanine dye, for example IR700, a targeting molecule (e.g., antibody or an antigen binding fragment of an antibody) that binds to a cell surface molecule, and a therapeutic agent. Also provided are compositions, article of manufacture, kits and methods for using the conjugates provided herein.

Photoimmunotherapy (PIT) is a molecular targeted therapy that utilizes a target-specific photosensitizer based on phthalocyanine dye, such as a near infrared (NIR) phthalocyanine dye (e.g., IR700), conjugated to a targeting molecule that targets a protein, such as a cell surface molecule on a cell in a disease, disorder or condition, such as a cell in a tumor. For example, in some cases a phthalocyanine dye-conjugate used in photoimmunotherapy can include conjugation to a monoclonal antibody (mAb) targeting a cell surface molecule receptor or receptor expressed on a cell in the environment of a disease lesion, such as a tumor microenvironment (TME), which can include tumor cells and other cells, such as immune cells. In some embodiments, activation of the dye-conjugate by irradiation with absorbing light, such as NIR light, excites the photosensitizer and results in cell killing, thereby reducing or eliminating the lesion (e.g., tumor) and treating the disease, disorder or condition. In some cases, the use of light in the NIR range leads to deeper tissue penetration resulting in successful eradication of tumors after only a single dose of external NIR light irradiation.

Generally, targeted phototoxicity is primarily dependent on binding of the dye-conjugate to the cell membrane via the specific targeting molecule (e.g., an antibody). For example, studies using an exemplary antibody-IR700 molecule indicate that the conjugate must be bound to the cellular membrane to be active, and that cell killing does not require intracellular localization to be effective (see, e.g., U.S. Pat. No. 8,524,239 and U.S. published application No. US20140120119). Photo-activation of the conjugate-bound cells results in rapid cell death and necrosis.

Typically, PIT results in cell death primarily of those cells to which the phthalocyanine-dye conjugate, such as IR700-antibody conjugate, binds after the cells are irradiated with NIR, while cells that do not express the cell surface molecule recognized by the targeting molecule (e.g., antibody) are not killed in significant numbers. Thus, because the therapy is targeted specifically to disease cells, such as cells in a tumor, its effects are highly selective to disease tissue compared to healthy tissue or cells. For example, although a targeted photosensitizer can be distributed throughout the body, it is only active where intense light is applied, reducing the likelihood of off-target effects. This is in contrast to non-PIT-based methods in which the activity of similar targeting molecules used as therapeutic agents (e.g., therapeutic antibodies) that are not conjugated to a photosensitizer cannot be localized, thereby resulting in significant risks of off-target side effects. Thus, PIT is an effective method of specifically targeting and killing disease cells or target lesions without substantially affecting healthy cells.

Improved strategies are needed for photoimmunotherapy methods, for example, to increase the efficacy of treatment and efficient delivery and targeting of additional therapeutic agents. For example, the efficacy of PIT can be reduced by the immunosuppressive environments of the lesion, e.g., tumor. The tumor microenvironment (TME) is generally immunosuppressive and can inhibit or hinder the anti-tumor activity of the immune cells. By targeting additional therapeutic agents that can help overcome such environments to particular sites, e.g., site of a lesion or a lesion associated with a disease, disorder or condition, the conjugates and methods provided herein can enhance the efficacy of PIT.

Cancerous cells contain tumor-specific antigens that should be recognized by the immune system. Typically, in an active immune system, immune cells, such as cytotoxic T cells, can attack, kill and/or eradicate these cancerous cells. Under normal physiological conditions, the T cell-mediated immune response is initiated by antigen recognition by the T cell receptor (TCR) and is regulated by a balance of co-stimulatory and inhibitory signals (e.g., immune checkpoint proteins). In particular, CD4+ and CD8+ T cells expressing a TCR can become activated upon recognition of antigenic peptides presented on antigen-presenting cells on major histocompatibility complex (MHC) class I or class II molecules, respectively. In some aspects, activated CD8+ cells, or cytotoxic T cells, can kill tumor cells expressing the antigen, which can be helped by the presence of CD4+ T cells. In some embodiments, the immune cell is an antigen presenting cell. In some embodiments, the immune cell is a dendritic cell.

In the case of lesions such as tumors, however, the TME has mechanisms to suppress the immune system, thereby evading immune recognition and preventing or reducing killing of tumor cells. For example, in some cases, immune checkpoint proteins can be dysregulated in tumors, thereby resulting in a suppression of the immune response in the TME as a mechanism of evading the immune system. In some cases, other mechanisms can act to inhibit access of immune cells to tumor antigens, thereby also contributing to the tumor's ability to evade the immune system. In some cases, existing therapies for tumors may not sufficiently address the immunosuppressive aspects of the TME.

In some cases, a combination therapy with an agent for PIT (e.g., phthalocyanine dye-antibody conjugate) and an additional therapy, e.g., an immune modulating agent or an anti-cancer agent, can be used to address some of the immunosuppressive effects of the TME and increase efficacy of the PIT. In some cases, however, the additional therapeutic agent is not targeted to the site or microenvironment of the lesion. Thus, the efficacy of the combination therapy may be reduced due to the lack of availability of the additional therapeutic agent at the site of the lesion. For example, anti-cancer agents that are administered generally or systemically may not be available at the site of the tumor for immediate uptake by the tumor cells in the TME.

In some aspects, the provided dual conjugates exploit the cytotoxic killing and/or lysis effects induced by PIT to enhance therapeutic outcomes in connection with tumor therapy, and can exploit binding of the targeting molecule to a cell surface molecule present in the microenvironment of the lesion, e.g., tumor antigen, to specifically target delivery of an additional therapeutic agent and maximize therapeutic efficacy of the therapeutic agent and/or PIT. In particular aspects, the dual conjugates contain one or more therapeutic agents that can be targeted or delivered to the site or microenvironment of the lesion. In some embodiments, such therapeutic agents include immune modulating agents that can boost or augment the activity of the immune cells in the TME. In other embodiments, such therapeutic agents include anti-cancer agents. Thus, the dual conjugates provided herein can effectively and efficiently activate specific killing of disease cells and also provide a boost or augmentation of immune activity or anti-cancer activity at the site of a lesion associated with the disease.

In some embodiments of the dual conjugates provided herein, the therapeutic agent is an immune modulating agent that inhibits immunosuppressive signaling or enhances immunostimulant signaling. For example, inhibitory checkpoint protein antagonists and/or agonists of co-stimulatory receptors can stimulate a host's endogenous anti-tumor immune response by amplifying antigen-specific T cell responses. In aspects of the provided dual conjugates and related methods, photoimmunotherapy also can be performed, which can result in the killing of tumor cells, thereby releasing tumor antigens and augmenting the anti-tumor immune response. By performing photoimmunotherapy with a dual conjugate containing an immune modulating agent, the release of PIT-induced antigens can provide a source of antigenic stimuli for the T cells whose response has been amplified or stimulated by the immune modulating agent. In some aspects, the enhanced immune response that is generated upon therapy with an immune modulating agent is primed and ready to respond to tumor antigens that are exposed upon lysis of cells after PIT. Thus, in some aspects, the dual conjugates provided herein address the natural evasion mechanisms that can be present in a tumor microenvironment, in order to provide a more robust immune response against the tumor while also killing tumor cells by photolytic mechanisms.

The dual conjugates and methods of using the dual conjugates provided herein address immune evasion mechanisms of tumors, in order to provide a more robust immune response against the tumor while also specifically targeting tumor cells by photolytic mechanisms, and also allow specific targeting of any additional therapeutic agent to be efficiently delivered to the site of the tumor. By combining the specific phototoxic killing of tumor cells and efficient delivery of therapeutic agents, such as immunomodulatory agents or anti-cancer agents, to the site or microenvironment of the lesion, the dual conjugates and related methods provided herein can improve the efficacy and safety of tumor therapy, and in some cases, increase the therapeutic outcome or survival of the treated subject.

For example, in contrast to combination therapy methods where a therapeutic agent is delivered systemically and requires separate administration of the therapeutic agent(s), the instant method permits rapid and effective delivery of the additional therapeutic agent to the site or microenvironment of the lesion, and reduces any lag time required in achieving a therapeutic effect. Because the additional therapeutic agent, e.g., immune modulating agent or anti-cancer agent, is available for direct and immediate uptake into the tumor space, the therapeutic response to the therapeutic agent can be maximized, in particular, with the activation of PIT. In some embodiments, the enhanced therapeutic outcome from the dual conjugate therapy can result in an increased reduction in tumor size (e.g., tumor volume or weight) or an increased or longer survival of the subject compared to methods involving treatment with either PIT or therapy with the additional therapeutic agent. Thus, in some embodiments, the therapeutic effect of the dual conjugate can be synergistic compared to that of treatment methods involving treatment with the phthalocyanine dye-conjugate/PIT or treatments involving the additional therapeutic agent, such as treatments with only the immune modulating agent or only the anti-cancer agent.

### I. DUAL CONJUGATES FOR PHOTOIMMUNOTHERAPY

Provided herein are conjugates, e.g., dual conjugates, containing a photosensitizer, such as a phthalocyanine dye, for example, IR700, a targeting molecule (e.g., antibody or an antigen binding fragment of an antibody) that binds to a cell surface molecule, and a therapeutic agent. In some embodiments, the dual conjugate contains a phthalocyanine dye, a targeting molecule and a therapeutic agent.

In some embodiments, the targeting molecule is capable of binding a cell surface molecule on a cell in a microenvironment of a lesion. In some embodiments, binding of the targeting molecule in the dual conjugate to the cell surface molecule permits the targeting of the dual conjugate to cells involved in a disease, disorder or condition, such as a tumor or cancer, infection, inflammatory disease or condition, neuronal disease or condition or other diseases or conditions. In some embodiments, the targeted cells (e.g., cells expressing the cell surface molecule capable of being bound by the targeting molecule) are present in the microenvironment of a lesion associated with the disease, disorder or condition, for example, the cells are present in a tumor microenvironment. In some embodiments, cell targeting increases the efficacy of photoimmunotherapy (PIT) induced upon local irradiation of the lesion (e.g., tumor) of the subject at a wavelength that is absorbed by the phthalocyanine dye (e.g., a near-infrared (NIR) wavelength), since cell killing is selective to those cells in which the dual conjugate is bound.

In some embodiments, the dual conjugate contains a therapeutic agent, such as an immune modulating agent or an anti-cancer agent. In some embodiments, the therapeutic agent is targeted or delivered to the site of the lesion, e.g., via the binding of the targeting molecule to the cell surface molecule. In some embodiments, the therapeutic agent is linked to the phthalocyanine dye or the targeting molecule via a releasable or cleavable linker, and release or cleavage of the linker permits release of the therapeutic agent from the dual conjugate. Thus, the therapeutic agent can be targeted or delivered directly to the cells involved in a disease, disorder or condition and/or be released into the microenvironment of a lesion associated with the disease, disorder or condition.

In some embodiments, the dual conjugate comprises the following components: (phthalocyanine dye)n, (targeting molecule)q and (therapeutic agent)m, wherein: n, q and m, which are selected independently, are at least 1. In some embodiments, n and q, which are selected independently, are between 1 and 10, such as between 1 and 9, between 1 and 8, between 1 and 7, between 1 and 6, between 1 and 5, such as 1, 2, 3, 4, 5, 6, 7, 8, 9 or 10. In some embodiments, n and q, which are selected independently, are 1 to 5. In some embodiments, n and m, which are selected independently, are between 1 and 10, such as between 1 and 9, between 1 and 8, between 1 and 7, between 1 and 6, between 1 and 5, such as 1, 2, 3, 4, 5, 6, 7, 8, 9 or 10. In some embodiments, n and m, which are selected independently, are 1 to 5. In some embodiments, q is 1, n is between 1 and 100, and m is between 1 and 5. In some embodiments, the ratio of n to q is from or from about 1 to about 1000, from or from about 1 to about 10 or from or from about 2 to about 5. In some embodiments, the targeting molecule is contacted with the phthalocyanine dye at a molar ratio of dye to targeting molecule from 1:1 to 100:1 or 1:1 to 10:1. In some embodiments, the molar ratio of dye to targeting molecule is at least or at least about 4:1 or is at least or at least about 10:1. In some embodiments, the dual conjugate includes from or from about 1 to about 1000 phthalocyanine dye molecules per targeting molecule, from or from about 1 to about 10 phthalocyanine dye molecules per targeting molecule or from or from about 2 to about 5 phthalocyanine dye molecules per targeting molecule. In some embodiments, the ratio of m to q is from or from about 1 to about 10 or from or from about 2 to about 5.

In some embodiments, the dual conjugate contains a number of dye residues per targeting molecule that is from or from about 1 to about 1000, such as from or from about 1 to about 100, from or from about 1 to about 50, from or from about 1 to about 25, from or from about 1 to about 10, from or from about 1 to about 5. In some embodiments, the ratio of dye molecules to targeting molecule is or is about 2:1, 3:1, 4:1, 5:1, 10:1, 15:1, 20:1, 25:1, 50:1, 75:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1 or 1000:1, or is between or between about any two of such values. In some embodiments, the targeting molecule may contain up to 2, 3, 4, 5, 6, 7, 8, 9, 10, 15, 20, 50, 75, 100, 150, 200, 250, 300, 350, 400, 450, 500, 550, 600, 650, 700, 750, 800, 850, 900, 950 or 1000 dye molecules. In some embodiments, the targeting molecule may contain more than 1000 dye molecules or less than 10 dye molecules.

In some embodiments, the dual conjugate contains a number of therapeutic agents per targeting molecule that is from or from about 1 to about 100, such as from or from about 1 to about 50, from or from about 1 to about 25, from or from about 1 to about 10, from or from about 1 to about 5. In some embodiments, the ratio of therapeutic agents to targeting molecule is or is about 2:1, 3:1, 4:1, 5:1, 10:1, 15:1, 20:1, 25:1, 50:1, 75:1 or 100:1, or is between or between about any two of such values. In some embodiments, the targeting molecule may contain up to 2, 3, 4, 5, 6, 7, 8, 9, 10, 15, 20, 50, 75 or 100 therapeutic agents. In some embodiments, the targeting molecule may contain more than 100 therapeutic agents or less than 10 therapeutic agents.

In some embodiments, the dual conjugate contains a number of dye residues per therapeutic agent that is from or from about 1 to about 1000, such as from or from about 1 to about 100, from or from about 1 to about 50, from or from about 1 to about 25, from or from about 1 to about 10, from or from about 1 to about 5. In some embodiments, the ratio of dye molecules to therapeutic agent is or is about 2:1, 3:1, 4:1, 5:1, 10:1, 15:1, 20:1, 25:1, 50:1, 75:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1 or 1000:1, or is between or between about any two of such values. In some embodiments, the therapeutic agent may contain up to 2, 3, 4, 5, 6, 7, 8, 9, 10, 15, 20, 50, 75, 100, 150, 200, 250, 300, 350, 400, 450, 500, 550, 600, 650, 700, 750, 800, 850, 900, 950 or 1000 dye molecules. In some embodiments, the therapeutic agent may contain more than 1000 dye molecules or less than 10 dye molecules.

In some embodiments, the dual conjugate contains a number of therapeutic agent per dye molecule that is from or from about 1 to about 1000, such as from or from about 1 to about 100, from or from about 1 to about 50, from or from about 1 to about 25, from or from about 1 to about 10, from or from about 1 to about 5. In some embodiments, the ratio of therapeutic agent to dye molecule is or is about 2:1, 3:1, 4:1, 5:1, 10:1, 15:1, 20:1, 25:1, 50:1, 75:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1 or 1000:1, or is between or between about any two of such values. In some embodiments, the dye molecule may contain up to 2, 3, 4, 5, 6, 7, 8, 9, 10, 15, 20, 50, 75, 100, 150, 200, 250, 300, 350, 400, 450, 500, 550, 600, 650, 700, 750, 800, 850, 900, 950 or 1000 dye therapeutic agents. In some embodiments, the dye molecule may contain more than 1000 therapeutic agents or less than 10 therapeutic agents.

In some embodiments, the components of the dual conjugates provided herein, e.g., the phthalocyanine dye, the targeting molecule and the therapeutic agent, can be linked in any order, each linkage being direct or indirect. In some embodiments, the phthalocyanine dye, the targeting molecule and the therapeutic agent can be linked via covalent or non-covalent linkage. In some aspects, the linkage is a cleavable linkage.

In some embodiments of the dual conjugates provided herein, the phthalocyanine dye and therapeutic agent are each independently linked to the targeting molecule. For example, in some embodiments, the dual conjugate comprises one of each components, in the order of phthalocyanine dye-targeting molecule-therapeutic agent. In some embodiments of the dual conjugates provided herein, the targeting molecule and therapeutic agent are each independently linked to the phythalocyanine dye. For example, in some embodiments, the dual conjugate comprises one of each components, in the order of targeting molecule-phthalocyanine dye-therapeutic agent. In some embodiments of the dual conjugates provided herein, the phythalocyanine dye and the targeting molecule are each independently linked to the therapeutic agent. For example, in some embodiments, the dual conjugate comprises one of each components, in the order of targeting molecule-therapeutic agent-phthalocyanine dye.

In some aspects, depending on the context and use of the dual conjugate, one type of molecule, e.g., a molecule that can specifically bind to or target another molecule and that also has therapeutic properties, may be considered either the therapeutic agent component or the targeting molecule component within the dual conjugate. In some embodiments, a molecule such as an antibody or antigen-binding fragment thereof or a cytokine, can be the targeting molecule component in the dual conjugate, with a different molecule as the therapeutic agent component in the dual conjugate. In some embodiments, a molecule such as an antibody or antigen-binding fragment thereof or a cytokine, can be the therapeutic agent component in the dual conjugate, with a different molecule as the targeting molecule component in the dual conjugate.

In some embodiments of the dual conjugate, a targeting molecule (e.g., an antibody or antigen-binding fragment thereof) is independently linked to a phthalocyanine dye (e.g., IR700) and a therapeutic agent (e.g., a cytokine or an anti-cancer agent). In some embodiments, an exemplary dual conjugate comprises an anti-HER1-IR700-therapeutic agent, such as cetuximab-IR700-IL-2.

A. Components of Conjugates

1. Phathalocyanine Dye

The provided dual conjugates contain a phthalocyanine dye, which can be linked, directly or indirectly, to one or both of the targeting molecule or the therapeutic agent. Phthalocyanines are a group of photosensitizer compounds having the phthalocyanine ring system. Phthalocyanines are azaporphyrins that contain four benzoindole groups connected by nitrogen bridges in a 16-membered ring of alternating carbon and nitrogen atoms (i.e., C₃₂H₁₆N₈) which form stable chelates with metal and metalloid cations. In these compounds, the ring center is occupied by a metal ion (either a diamagnetic or a paramagnetic ion) that may, depending on the ion, carry one or two ligands. In addition, the ring periphery may be either unsubstituted or substituted. The synthesis and use of a wide variety of phthalocyanines in photodynamic therapy are described in International Publication WO 2005/099689 and U.S. Pat. No. 7,005,518. In some embodiments, the phthalocyanine dye is conjugated to a targeting molecule and/or therapeutic agent via a reactive group of the dye molecule.

In some embodiments, phthalocyanines strongly absorb red or near IR radiation with absorption peaks falling between about 600 nm and 810 nm, which, in some cases, allow deep penetration of tissue by the light. Phthalocyanines are generally photostable. This photostability is typically advantageous in pigments and dyes and in many of the other applications of phthalocyanines.

In some embodiments, the phthalocyanine dye is water soluble and contains a luminescent fluorophore moiety having at least one aqueous-solubilizing moiety. In some embodiments, the aqueous solubilizing moiety contains silicon. In some embodiments, the phthalocyanine dye has a core atom such as Si, Ge, Sn, or Al. In some embodiments, the phthalocyanine dye exists as a single core isomer, essentially free of other isomers. In some embodiments, the phthalocyanine dye contains a linker that has a reactive or activatable group, which is able to form a bond between the linker and targeting molecule. In some embodiments, the phthalocyanine dye can be tailored to fluoresce at a particular wavelength.

In some embodiments, the phthalocyanine dye contains a linker, i.e., is a linker-phthalocyanine dye moiety (L-D). In some embodiments, the linker contains a reactive group. In some embodiments, the phthalocyanine dye is of Formula Ia:

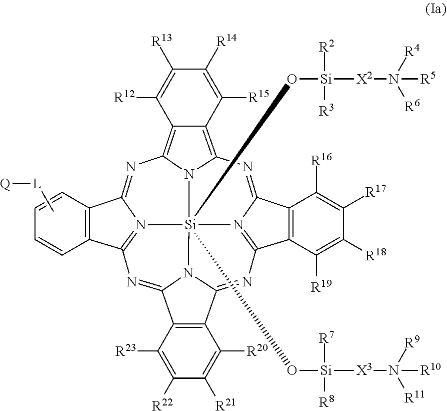

Wherein:

L is selected from a direct link, or a covalent linkage;

Q is a reactive group or an activatable group that can be part of the linker L, and is any group that can react to form a bond between L and the targeting molecule A;

R², R³, R⁷, and R⁸ are each independently selected from optionally substituted alkyl and optionally substituted aryl;

R⁴, R⁵, R⁶, R⁹, R¹⁰, and R¹¹, if present, are each independently selected from hydrogen, optionally substituted alkyl, optionally substituted alkanoyl, optionally substituted alkoxycarbonyl, optionally substituted alkylcarbamoyl, or a chelating ligand, wherein at least one of R⁴, R⁵, R⁶, R⁹, R¹⁰, and R¹¹ comprises a water soluble group;

R¹², R¹³, R¹⁴, R¹⁵, R¹⁶, R¹⁷, R¹⁸, R¹⁹, R²⁰, R²¹, R²² and R²³ are each functional groups that can be independently selected from hydrogen, halogen, optionally substituted alkylthio, optionally substituted alkylamino or optionally substituted alkoxy;

or in an alternative embodiment, at least one of i) R¹³ and R¹⁴, and the carbons to which they are attached, or ii) R¹⁷ and R¹⁸, and the carbons to which they are attached, or iii) R²¹ and R²², and the carbons to which they are attached, join to form a fused ring; and

X² and X³ are each independently C₁-C₁₀ alkylene, optionally interrupted by a heteroatom.

In some embodiments, L is a covalent linkage. In some embodiments, the covalent linkage is linear or branched, cyclic or heterocyclic, saturated or unsaturated, having 1-60 atoms, such as 1-45 atoms or 1-25 atoms. In some cases, such atoms can be selected from C, N, P, O, and S. In some embodiments, L can have additional hydrogen atoms to fill valences (in addition to the 1-60 atoms). Generally, the linkage contains any combination of ether, thioether, amine, ester, carbamate, urea, thiourea, oxy or amide bonds; or single, double, triple or aromatic carbon-carbon bonds; or phosphorus-oxygen, phosphorus-sulfur, nitrogen-nitrogen, nitrogen-oxygen, or nitrogen-platinum bonds; or aromatic or heteroaromatic bonds.

In some embodiments, L is of the formula —R¹—Y—X¹—Y¹—, wherein R¹ is a bivalent radical or direct link; Y and Y¹ are each independently selected from t a direct link, oxygen, an optionally substituted nitrogen, or sulfur; and X¹ is selected from t a direct link and C₁-C₁₀ alkylene optionally interrupted by an atom. Bivalent radicals include, but are not limited to, optionally substituted alkylene, optionally substituted alkyleneoxycarbonyl, optionally substituted alkylenecarbamoyl, optionally substituted alkylenesulfonyl, and optionally substituted arylene.

Exemplary R¹ substituents include, but are not limited to, optionally substituted alkylene, optionally substituted alkyleneoxycarbonyl, optionally substituted alkylenecarbamoyl, optionally substituted alkylenesulfonyl, optionally substituted alkylenesulfonylcarbamoyl, optionally substituted arylene, optionally substituted arylenesulfonyl, optionally substituted aryleneoxycarbonyl, optionally substituted arylenecarbamoyl, optionally substituted arylenesulfonylcarbamoyl, optionally substituted carboxyalkyl, optionally substituted carbamoyl, optionally substituted carbonyl, optionally substituted heteroarylene, optionally substituted heteroaryleneoxycarbonyl, optionally substituted heteroarylenecarbamoyl, optionally substituted heteroarylenesulfonylcarbamoyl, optionally substituted sulfonylcarbamoyl, optionally substituted thiocarbonyl, a optionally substituted sulfonyl, and optionally substituted sulfinyl.

In some embodiments, Q contains a reactive group for optional attachment to a material, such as a targeting molecule. As used herein, the term “reactive group” means a moiety on the compound that is capable of chemically reacting with the functional group on a different material (e.g., targeting molecule) to form a linkage, such as a covalent linkage. Typically, the reactive group is an electrophile or nucleophile that can form a covalent linkage through exposure to the corresponding functional group that is a nucleophile or electrophile, respectively. Alternatively, the reactive group is a photoactivatable group, and becomes chemically reactive only after illumination with light of an appropriate wavelength. Typically, the conjugation reaction between the reactive dye and the targeting molecule to be conjugated results in one or more atoms of the reactive group Q incorporated into a new linkage attaching the dye to the conjugated targeting molecule and/or therapeutic agent.

In some embodiments, Q contains a reactive group that is reactive with a carboxyl group, an amine, or a thiol group on the targeting molecule. Suitable reactive groups include, but are not limited to, an activated ester, an acyl halide, an alkyl halide, an anhydride, a carboxylic acid, a carbodiimide, a carbonate, a carbamate, a haloacetamide (e.g., iodoacetamide), an isocyanate, an isothiocyanate, a maleimide, an NHS ester, a phosphoramidite, a platinum complex, a sulfonate ester and a thiocyanate for optional attachment to the targeting molecule. In some embodiments, the reactive groups are reactive with a carboxyl group, an amine, or a thiol group on a targeting molecule. In some embodiments, the reactive group is a sulfhydryl-reactive chemical group such as maleimide, haloacetyl, and pyridyl disulfide. In some embodiments, the reactive group is amine-reactive. In some embodiments, the reactive group is an NHS ester.

In some embodiments, R², R³, R⁷, and R⁸ are each optionally substituted alkyl such as optionally substituted methyl, ethyl, or isopropyl groups.

In some embodiments, at least one of R⁴, R⁵, R⁶, R⁹, R¹⁰, and R¹¹ contains a water soluble group. For example, the alkyl portion of R⁴, R⁵, R⁶, R⁹, R¹⁰, and R¹¹ is substituted with a water soluble substituent. As used herein, “water soluble group” refers to a group comprising one or more polar and/or ionic substituents that improves the solubility of the overall molecule in aqueous media. In some cases, at least two of R⁴, R⁵, R⁶, R⁹, R¹⁰, and R¹¹ comprise water soluble groups. In other embodiments, three or more comprise water soluble groups. Water soluble groups include, but are not limited to, a carboxylate (—CO₂⁻) group, a sulfonate (—SO₃⁻) group, a sulfonyl (—SO₂⁻) group, a sulfate (—SO₄⁻²) group, a hydroxyl (—OH) group, a phosphate (—OPO₃⁻²) group, a phosphonate (—PO₃⁻²) group, an amine (—NH₂) group and an optionally substituted quaternized nitrogen with each having an optional counter ion.

Suitable counter ions include, but are not limited to, sodium, potassium, calcium, ammonium, organic amino salt, or magnesium salt, or a similar salt. Preferably, the counter ion is a biologically acceptable counter ion.

In some embodiments, the nitrogen atom(s) to which R⁴, R⁵, R⁶, R⁹, R¹⁰, and R¹¹ are attached can be trivalent or tetravalent.

In some embodiments, R¹², R¹³, R¹⁴, R¹⁵, R¹⁶, R¹⁷, R¹⁸, R¹⁹, R²⁰, R²¹, R²² and R²³ are each hydrogen.

In some embodiments, X² and X³ are each independently selected from C₁-C₁₀ alkylene optionally interrupted by an atom. In some embodiments, the nitrogens appended to X² and/or X³ can be optionally quaternized.

In some embodiments, the phthalocyanine dye is of Formula Ib:

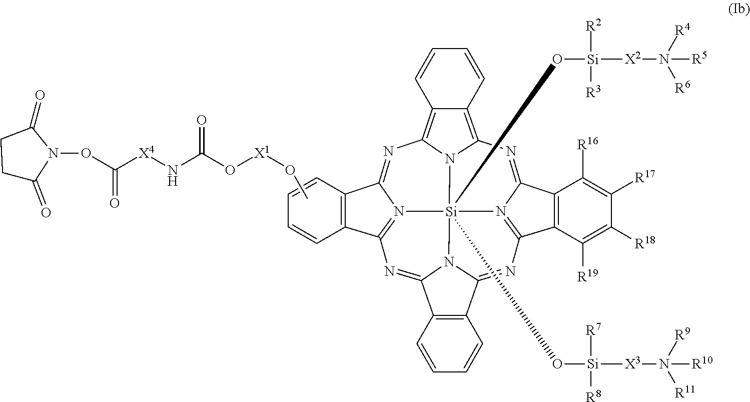

wherein

X¹ and X⁴ are each independently a C₁-C₁₀ alkylene optionally interrupted by a heteroatom; and

R², R³, R⁴, R⁵, R⁶, R⁷, R⁸, R⁹, R¹⁰, R¹¹, R¹⁶, R¹⁷, R¹⁸, R¹⁹, X², and X³ are as defined herein.

In some embodiments, the reactive group is an NHS ester. In some embodiments, the reactivity of the NHS ester can be adjusted by varying the length of the alkylene group of X⁴, between the NHS ester and carbamate functionality. In some embodiments, the length of the alkylene group of X⁴ between the NHS ester and the carbamate functionality is inversely proportional to the NHS ester reactivity. In some embodiments, X⁴ is C₅-alkylene. In other embodiments, X⁴ is C₃-alkylene. In some embodiments, X¹ is C₆-alkylene. In other embodiments, X¹ is C₃-alkylene.

In some embodiments, the phthalocyanine dye has an overall electronic charge of zero. This charge neutrality can in certain instances by obtained with one or more optional counterions, or quaternized nitrogens.

In some embodiments, the phthalocyanine dye has sufficient solubility in aqueous solutions that once it is attached to a soluble targeting molecule, the targeting molecule retains its solubility. In some embodiments, the dye also is soluble in organic media (e.g., DMSO or DMF).

In some embodiments, the phthalocyanine dye has a maximum light absorption in the near infrared (NIR range). In some embodiments, the phthalocyanine dye has a maximum light absorption wavelength between 400 nm and 900 nm, such as between 600 nm and 850 nm, such as between 680 nm and 850 nm, for example at approximately 690 nm ±50 nm or 690 ±20 nm. In some embodiments, the phthalocyanine dye can be excited efficiently by commercially available laser diodes that emit light at these wavelengths.

In some embodiments, the phthalocyanine dye containing the reactive group is IR700 NHS ester, such as IRDye 700DX NHS ester (Li-Cor 929-70010, 929-70011). Thus, in some embodiments, the dye is a compound having the following formula:

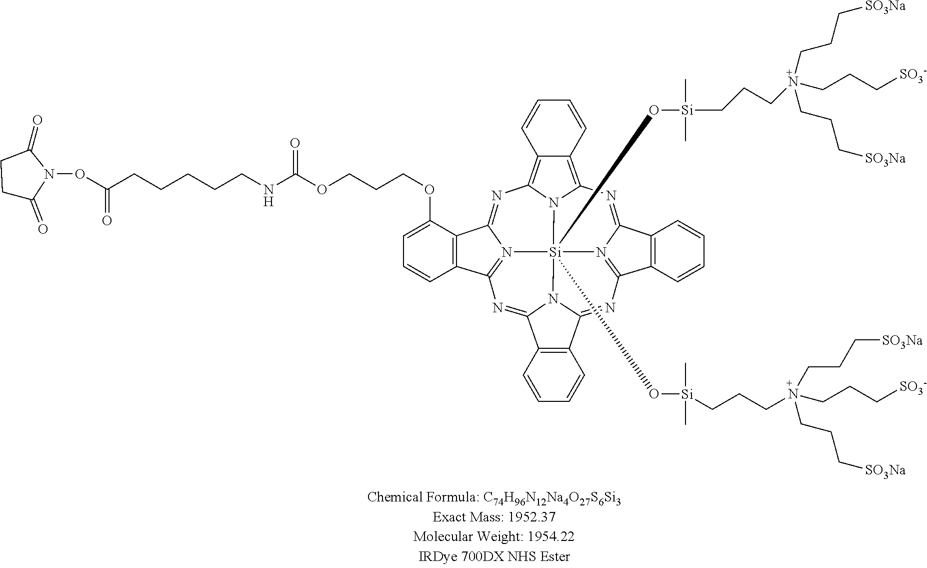

For purposes herein, the term “IR700,” “IRDye 700DX,” or variations thereof refer to the above formula when the dye is conjugated to a targeting molecule via its reactive group. Generally, IR700 has several favorable chemical properties. Amino-reactive IR700 is a relatively hydrophilic dye and can be covalently conjugated with an antibody using the NHS ester of IR700. Typically, IR700 also has more than 5-fold higher extinction coefficient (2.1×10⁵ M⁻¹cm⁻¹ at the absorption maximum of 689 nm), than conventional photosensitizers such as the hematoporphyrin derivative Photofrin® (1.2×10³ M⁻¹cm⁻¹ at 630 nm), meta-tetrahydroxyphenylchlorin; Foscan® (2.2×10⁴ M⁻¹cm⁻¹ at 652 nm), and mono-L-aspartylchlorin e6; NPe6/Laserphyrin® (4.0×10⁴M⁻¹cm⁻¹ at 654 nm).

The phthalocyanine dyes described herein can be made with commercially available starting material. The core structure is synthesized by condensation of two or more different diiminoisoindolines. Synthetic strategies using different dinitriles or diiminoisoindolines can lead to various degrees of substitution of the phthalocyanine and/or distribution of regioisomers. Exemplary synthetic schemes for generating the dyes are described in U.S. Pat. No. 7,005,518.

In some embodiments, the dual conjugate can comprise one or more phthalocyanine dyes, and the one or more phthalocyanine dyes can be the same or different.

2. Targeting Molecule

The provided dual conjugates contain a targeting molecule, which can be linked, directly or indirectly, to one or both of the phthalocyanine dye or the therapeutic agent. In some embodiments, the targeting molecule is one that is able to target the dual conjugate to a cell or pathogen, for example, by binding to a cell surface molecule (e.g. cell surface receptor) on the cell or pathogen. In some embodiments, the targeting molecule is an antibody or antigen-binding fragment thereof. In some embodiments, the targeting molecule, e.g., an antibody or antigen-binding fragment thereof, can selectively bind to a desired cell type, cells with a particular phenotype, or cells displaying one or more cell surface markers or antigens. In some cases, the targeting molecule binds to a cell that is a cancer cell, a tumor cell, an inflammatory cell, an immune cell, a neuron, a stem cell, a proliferating cell, or a cell in a hyperplasia. In some cases, the targeting molecule binds to a pathogen or a pathogen infected cell. In some embodiments, the cell is an inflammatory cell, such a leukocyte, for example, a neutrophil, an eosinophil, a basophil, a lymphocyte, or a monocyte. In some embodiments, the cell is an immune cell, such as a T cell, a B cell, a Natural Killer (NK) cell, a dendritic cell, a macrophage or a neutrophil. In some embodiments, the cell is a neuron that is a peripheral nervous system neuron or a central nervous system neuron, such as a nociceptor, for example, thermal nociceptors, mechanical nociceptors, chemical nociceptors or polymodal nociceptors. In some cases, the targeting molecule binds to a pathogen or a pathogenic cell, such as a virus, bacterium, fungus, biofilm or other prokaryotic cell system. In some embodiments, the targeting molecule binds to a pathogen that is a gram-negative or gram-positive bacterium.

In some embodiments, the targeting molecule (e.g., antibody) of the dual conjugate bind to a protein on the surface of a cell or cells present in a microenvironment of a lesion that is associated with or present as a result of a disease, disorder or condition. For example, in some embodiments, the dual conjugate binds to a protein on the surface of a cell or cells present in a tumor microenvironment associated with or present in a tumor. In some embodiments, the dual conjugate binds to a protein present the extracellular matrix in the microenvironment of the tumor.

In some embodiments, the targeting molecule itself also can be an agent used in therapy or treatment of a disease, disorder or condition. In some embodiments, the targeting molecule also can mediate a therapeutic effect. In some embodiments, the targeting molecule is also an agent used in therapy or treatment of a disease, disorder or condition by binding to a protein on the surface of a cell or cells present in a microenvironment of a lesion that is associated with or present as a result of a disease, disorder or condition. In some embodiments, the targeting molecule is an antibody or antigen-binding fragment thereof that binds to a cell surface protein present in a microenvironment of a lesion. In some embodiments, the targeting molecule is an antibody or antigen-binding fragment thereof that binds to an immunologic target, such as a cell surface receptor expressed on immune cells or cell surface proteins involved in immune modulation. In some aspects, the targeting molecule is an immune modulating agent, such as an immune checkpoint inhibitor or a cytokine. In some aspects, the targeting molecule itself can be an agent selected from those described in Section I.A.3 below, such as an immune modulating agent or an anti-cancer agent. In some aspects, depending on the context and use of the dual conjugate, one type of molecule, e.g., a molecule that can specifically bind to or target another molecule and that also has therapeutic properties, may be considered either the therapeutic agent component or the targeting molecule component within the dual conjugate.

As used herein, a “cell present in the microenvironment of a lesion” refers to any cell present in the cellular environment associated with a lesion, a disease a disorder or a condition, such as any cell present in or immediately adjacent to a tumor, such as cells present in a tumor microenvironment (TME), or the extracellular matrix in the tumor microenvironment.

As used herein, a “cell present in a tumor microenvironment” or a “cell present in the TME” refers to any cell present in the cellular environment in which the tumor exists, such as any cell present in or immediately adjacent to the tumor, including the proliferating tumor cells (e.g., cancer cells), the tumor stroma, blood vessels, infiltrating inflammatory cells (e.g., immune cells) and a variety of associated tissue cells (e.g., fibroblasts). Thus, it is understood that reference to the tumor refers not only to the tumor cells, which can include malignant or cancer cells, but also to other cells present in the tumor microenvironment that regulate the growth of the tumor, including immune cells. In some cases, immune cells present in a tumor microenvironment can include T lymphocytes, including regulatory T lymphocytes (Treg), dendritic cells, natural killer (NK) cells, B cells, macrophages and other immune cells (Whiteside (2008) Oncogene, 27:5904-5912). It is recognized that, in some aspects, many non-cancerous cells present in and around the tumor can regulate the proliferation, angiogenesis, invasion and/or metastasis of tumor cells, thereby promoting the growth of the tumor. Thus, in some cases, targeting such non-cancerous cells, such as immune cells (e.g., T cells, such as regulatory T cells), present in a tumor can be an effective therapy for killing a tumor by PIT.

Generally, cancerous cells contain antigens associated with a tumor that should be recognized by the immune system. Typically, in an active immune system, immune cells, such as cytotoxic T cells, attack and eradicate these cancerous cells. Under normal physiological conditions, the T cell-mediated immune response is initiated by antigen recognition by the T cell receptor (TCR) and is regulated by a balance of co-stimulatory and inhibitory signals (e.g., immune checkpoint proteins). In particular, CD4+ and CD8+ T cells expressing a TCR can become activated upon recognition of antigenic peptides presented on antigen-presenting cells on major histocompatibility complex (MHC) class I or class II molecules, respectively. In some aspects, activated CD8+ cells, or cytotoxic T cells, can kill tumor cells expressing the antigen, which can be helped by the presence of CD4+ T cells.

In the case of tumors, however, the tumor microenvironment (TME) has mechanisms to suppress the immune system, thereby evading immune recognition and preventing or reducing killing of tumor cells. For example, in some cases, immune checkpoint proteins can be dysregulated in tumors, thereby resulting in a suppression of the immune response in the tumor microenvironment as a mechanism of evading the immune system. In some cases, tumor-infiltrating lymphocytes can include Tregs (e.g., CD4+CD25+ T cells), which are cells that are capable of suppressing proliferation of other T cells in the microenvironment (Whiteside, T L (2008) Oncogene, 27:5904-5912). In some cases, other mechanisms can act to inhibit access of immune cells to tumor antigens, thereby also contributing to the tumor's ability to evade the immune system.

In some embodiments, the targeting molecule is a targeting molecule that binds to a cell surface molecule on a tumor or cancer cell. In some embodiments, the targeting molecule binds to a cell surface molecule on an immune cell or other non-cancerous cell present in a tumor microenvironment. In some embodiments, the targeting molecule binds to a cell surface molecule on the surface of a T lymphocyte, such as a Treg, a dendritic cell, a natural killer (NK) cell, a B cell, a macrophage or other immune cell that is present in a tumor microenvironment. In some cases, the tumor or cancer is located at the head and neck, breast, liver, colon, ovary, prostate, pancreas, brain, cervix, bone, skin, eye, bladder, stomach, esophagus, peritoneum, or lung.

Exemplary of targeting molecules, such as targeting molecules that target a tumor or cancer or a tumor associated with a cancer, include, but are not limited to, any as described in published international PCT appl. Nos. WO2014120974, WO2014176284, WO2015042325, U.S. Pat. No. 8,524,239 or U.S. patent publication No. US20140120119.

Exemplary targeting molecules include, but are not limited to, a protein, a glycoprotein, an antibody, an antibody fragment, an antigen, an antigen binding fragment, a peptide, a polypeptide, a tissue homing peptide, a small molecule, a polymeric synthetic molecule, a polymeric nanoparticle, a liposome, an enzyme substrate, a hormone, a neurotransmitter, a cell metabolite, a viral particle, a viral capsid, a viral nanoparticle, a bacterial particle, a marker, a cell, a hapten, an avidin, a streptavidin, a monomeric streptavidin, a biotin, a carbohydrate, an oligosaccharide, a polysaccharide, a nucleic acid, a deoxy nucleic acid, a fragment of DNA, a fragment of RNA, an aptamer, nucleotide triphosphates, acyclo terminator triphosphates, PNA or a combination thereof.

In some embodiments, the targeting molecule is an amino acid, peptide, protein, tyramine, polysaccharide, a small molecule, ion-complexing moiety, nucleoside, nucleotide, oligonucleotide, psoralen, drug, hormone, lipid, lipid assembly, polymer, polymeric microparticle, a biological cell, or virus, or any combination thereof. In some embodiments, the targeting molecule is an antigen, steroid, vitamin, drug, metabolite, toxin, environmental pollutant, nucleic acid polymer, carbohydrate, lipid, or glass, plastic or other non-biological polymer or any combination thereof. In some embodiments, the targeting molecules is a cell, cellular system, cellular fragment, or subcellular particle, e.g., a virus particle, bacterial particle, virus component, biological cell (such as animal cell, plant cell, bacteria, yeast, or protist), or cellular component or any combination thereof. In some embodiments, reactive dyes may label functional groups at the cell surface, in cell membranes, organelles, or cytoplasm or any combination thereof.

In some embodiments, the targeting molecule targets or binds to an antigen, such as any structural substance that serves as a target capable of being bound by the targeting molecule. In some embodiments, the antigen is or is comprised as part of a cell surface molecule, such as a protein, e.g., a receptor, that is expressed on a cell surface. In some embodiments, for example, the antigen is or is comprised as part of a molecule expressed on the surface of a cell present in a tumor, including any cell present in the tumor microenvironment. Examples of cell surface molecules include, but are not limited to, an antigen, peptides, lipids, polysaccharides, carbohydrate, or nucleic acids containing antigenic determinants or any combination thereof, such as those recognized by an immune cell. In some examples, an antigen includes a tumor-specific peptide (such as one found on the surface of a cancer cell) or immunogenic fragment thereof. In some embodiments, the targeting molecule is an antibody or an antigen-binding antibody fragment thereof.

In some embodiments, the cell surface molecule can be ACTHR, endothelial cell Anxa-1, aminopetidase N, anti-IL-6R, alpha-4-integrin, alpha-5-beta-3 integrin, alpha-5-beta-5 integrin, alpha-fetoprotein (AFP), ANPA, ANPB, APA, APN, APP, 1AR, 2AR, AT1, B1, B2, BAGE1, BAGE2, B-cell receptor BB1, BB2, BB4, calcitonin receptor, cancer antigen 125 (CA 125), CCK1, CCK2, CD5, CD10, CD11a, CD13, CD14, CD19, CD20, CD22, CD25, CD30, CD33, CD38, CD45, CD52, CD56, CD68, CD90, CD133, CD7, CD15, CD34, CD44, CD206, CD271, CEA (CarcinoEmbryonic Antigen), CGRP, chemokine receptors, cell-surface annexin-1, cell-surface plectin-1, Cripto-1, CRLR, CXCR2, CXCR4, DCC, DLL3, E2 glycoprotein, EGFR, EGFRvIII, EMR1, Endosialin, EP2, EP4, EpCAM, EphA2, ET receptors, Fibronectin, Fibronectin ED-B, FGFR, frizzled receptors, GAGE1, GAGE2, GAGE3, GAGE4, GAGE5, GAGE6, GLP-1 receptor, G-protein coupled receptors of the Family A (Rhodopsin-like), G-protein coupled receptors of the Family B (Secretin receptor-like) like), G-protein coupled receptors of the Family C (Metabotropic Glutamate Receptor-like), GD2, GP100, GP120, Glypican-3, hemagglutinin, Heparin sulfates, HER1, HER2, HER3, HER4, HMFG, HPV 16/18 and E6/E7 antigens, hTERT, IL11-R, IL-13R, ITGAM, Kalikrien-9, Lewis Y, LH receptor, LHRH-R, LPA1, MAC-1, MAGE 1, MAGE 2, MAGE 3, MAGE 4, MART 1, MC1R, Mesothelin, MUC1, MUC16, Neu (cell-surface Nucleolin), Neprilysin, Neuropilin-1, Neuropilin-2, NG2, NK1, NK2, NK3, NMB-R, Notch-1, NY-ESO-1, OT-R, mutant p53, p97 melanoma antigen, NTR2, NTR3, p32 (p32/gC1q-R/HABP1), p′75, PAC1, PAR1, Patched (PTCH), PDGFR, PDFG receptors, PDT, Protease-cleaved collagen IV, proteinase 3, prohibitin, protein tyrosine kinase 7, PSA, PSMA, purinergic P2X family (e.g., P2X1-5), mutant Ras, RAMP1, RAMP2, RAMP3 patched, RET receptor, plexins, smoothened, sstl, sst2A, sst2B, sst3, sst4, sst5, substance P, TEMs, T-cell CD3 Receptor, TAG72, TGFBR1, TGFBR2, Tie-1, Tie-2, Trk-A, Trk-B, Trk-C, TR1, TRPA, TRPC, TRPV, TRPM, TRPML, TRPP (e.g., TRPV1-6, TRPA1, TRPC1-7, TRPM1-8, TRPP1-5, TRPML1-3), TSH receptor, VEGF receptors (VEGFR1 or Flt-1, VEGFR2 or FLK-1/KDR, and VEGF-3 or FLT-4), voltage-gated ion channels, VPAC1, VPAC2, Wilms tumor 1, Y1, Y2, Y4, or Y5.

In some embodiments, the targeting molecule is a binding partner, such as a ligand, capable of binding to a cell surface molecule, such as a cell surface molecule, e.g., a cell surface receptor. In some embodiments, the targeting molecule is selected from adrenocorticotropic hormone (ACTH), angiotensin II, atrial natriuretic factor (ANF), bombesin, bradykinin, brain derived neurotropihic factor (BDNF), bone morphogenetic protein 2 (BMP-2), bone morphogenetic protein 6 (BMP-6), bone morphogenetic protein 7 (BMP-7), calcitonin, cardiotrophin 1 (BMP-2), CD22, CD40, cholecystokinin (CCK), ciliary neurotrophic factor (CNTF), CCL1-CCL28, CXCL1-CXCL17, XCL1, XCL2, CX3CL1, cripto 1 binding peptide, vascular endothelial cell growth factor (VEGF), epidermal growth factor (EGF), endothelin 1, endothelin 1/3, FAS-ligand, fibroblast growth factor 1 (FGF-1), fibroblast growth factor 2 (FGF-2), fibroblast growth factor 4 (FGF-4), fibroblast growth factor 5 (FGF-5), fibroblast growth factor 6 (FGF-6), fibroblast growth factor 1 (FGF-7), fibroblast growth factor 1 (FGF-10), Flt-3, gastrin, gastrin releasing peptide (GRP), granulocyte colony-stimulating factor (G-CSF), granulocyte macrophage stimulating factor (GM-CSF), glucagon like peptide (GLP-1), hepatocyte growth factor (HGF), interferon alpha (IFN-a), interferon beta (IFN-b), interferon gamma (IFNg), insulin-like growth factor 1(IGF-1), insulin-like growth factor 2 (IGF-2), interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 15 (IL-15), interleukin 17 (IL-17), interleukin 19 (IL-19), luteinizing hormone (LH), luteinizing-releasing hormone (LHRH), macrophage colony-stimulating factor (M-CSF), monocyte chemotactic protein 1 (MCP-1), macrophage inflammatory protein 3a (MIP-3a), macrophage inflammatory protein 3b (MIP-3b), nerve growth factor (NGF), neuromedin B, neurotrophin 3 (NT-3), neurotrophin 4 (NT-4), neurotensin, neuropeptide Y, oxytocin, pituitary adenylate cyclase activating peptide (PACAP), platelet derived growth factor AA (PDGF-AA), platelet derived growth factor AB (PDGF-AB), platelet derived growth factor BB (PDGF-BB), platelet derived growth factor CC (PDGF-CC), platelet derived growth factor DD (PDGF-DD), netrin-1 (NTN1), netrin-2 (NTN2), netrin-4 (NTN4), netrin-G1 (NTNG1) and netrin-G2 (NTNG2), ephrin Al (EFNA1), ephrin A2 (EFNA2), ephrin A3 (EFNA3), ephrin A4 (EFNA4), ephrin A5 (EFNA5), semaphorin 3A (SEMA3A), semaphorin 3B (SEMA3B), semaphorin 3C (SEMA3C), semaphorin 3D (SEMA3D), semaphorin 3F (SEMA3F), semaphorin 3G (SEMA3G), semaphorin 4A (SEMA4A), semaphorin 4B (SEMA4B), semaphorin 4C (SEMA4C), semaphorin 4D (SEMA4D), semaphorin 4F (SEMA4F), semaphorin 4G (SEMA4G), semaphorin 5A (SEMA5A), semaphorin 5B (SEMA5B), semaphorin 6A (SEMA6A), semaphorin 6B (SEMA6B), semaphorin 6D (SEMA6D), semaphorin 7A (SEMA7A), SLIT1, SLIT2, SLIT3, SLIT and NTRK-like family, member 1 (SLITRK1), SLIT and NTRK-like family, member 2 (SLITRK2), SLIT and NTRK-like family, member 3 (SLITRK3), SLIT and NTRK-like family, member 4 (SLITRK4), SLIT and NTRK-like family, member 5 (SLITRK5), SLIT and NTRK-like family, member 6 (SLITRK6), prostaglandin E2 (PGE2), RANTES, Somatostatin-14, Somatostatin-28, stem cell factor (SCF), stromal cell derived factor 1 (SDF-1), substance P, thyroid stimulating hormone (TSH), transforming growth factor alpha (TGF-a), transforming growth factor beta (TGF-b), tumor necrosis factor alpha (TNF-α), thrombin, vasoactive intestinal peptide (VIP), Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16, Sonic hedgehog, Desert hedgehog, and Indian hedgehog, or is a binding fragment thereof that is capable of binding to its cognate cell surface molecule, such as a cell surface molecule, e.g., cell surface receptor.

In some embodiments, the targeting molecule can be an immune modulating agent, which can bind to a cell surface molecule or protein on an immune cell to either suppress or activate the body's immune response. In some embodiments, binding of the immune modulating agent to the cell surface molecule or protein can stimulate an immune response to a tumor and/or a pathogen, such as by inhibiting immune suppression or by enhancing immunostimulation. In some embodiments, the cell surface molecule or protein can be CD25, PD-1 (CD279), PD-L1 (CD274, B7-H1), PD-L2 (CD273, B7-DC), CTLA-4, LAG3 (CD223), TIM3 (HAVCR2), 4-1BB (CD137, TNFRSF9), CXCR2, CXCR4 (CD184), CD27, CEACAM1, Galectin 9, BTLA, CD160, VISTA (PD1 homologue), B7-H4 (VCTN1), CD80 (B7-1), CD86 (B7-2), CD28, HHLA2 (B7-H7), CD28H, CD155, CD226, TIGIT, CD96, Galectin 3, CD40, CD40L, CD70, LIGHT (TNFSF14), HVEM (TNFRSF14), B7-H3 (CD276), Ox40L (TNFSF4), CD137L (TNFSF9, GITRL), B7RP1, ICOS (CD278), ICOSL, KIR, GALS, NKG2A (CD94), GARP, TL1A, TNFRSF25, TMIGD2, BTNL2, Butyrophilin family, CD48, CD244, Siglec family, CD30, CSF1R, MICA (MHC class I polypeptide-related sequence A), MICB (MHC class I polypeptide-related sequence B), NKG2D, KIR family (Killer-cell immunoglobulin-like receptor, LILR family (Leukocyte immunoglobulin-like receptors, CD85, ILTs, LIRs), SIRPA (Signal regulatory protein alpha), CD47 (IAP), Neuropilin 1 (NRP-1), a VEGFR or VEGF. In some example, the targeting molecule is an antibody or antigen-binding fragment that is an immune modulating agent. In some embodiments, the immune modulating agent is an immune checkpoint inhibitor.

In some embodiments, the cell surface molecule can be HER1/EGFR, HER2/ERBB2, CD20, CD25 (IL-2Rα receptor), CD33, CD52, CD133, CD206, CEA, CEACAM1, CEACAM3, CEACAM5, CEACAM6, cancer antigen 125 (CA125), alpha-fetoprotein (AFP), Lewis Y, TAG72, Caprin-1, mesothelin, PDGF receptor, PD-1, PD-L1, CTLA-4, IL-2 receptor, vascular endothelial growth factor (VEGF), CD30, EpCAM, EphA2, Glypican-3, gpA33, mucins, CAIX, PSMA, folate-binding protein, gangliosides (such as GD2, GD3, GM1 and GM2), VEGF receptor (VEGFR), integrin αVβ3, integrin α5β1, ERBB3, MET, IGF1R, EPHA3, TRAILR1, TRAILR2, RANKL, FAP, tenascin, AFP, BCR complex, CD3, CD18, CD44, CTLA-4, gp72, HLA-DR 10 β, HLA-DR antigen, IgE, MUC-1, nuC242, PEM antigen, metalloproteinases, Ephrin receptor, Ephrin ligands, HGF receptor, CXCR4, CXCR4, Bombesin receptor, or SK-1 antigen.

In some embodiments, the targeting molecule is an antibody or an antigen-binding antibody fragment that specifically binds to an antigen that is or is part of a cell surface molecule expressed on the surface of a cell. Included among such antibodies are antibodies or antigen-binding antibody fragments capable of binding to a cell surface molecule, such as a cell surface molecule, e.g., cell surface receptor, described herein. In some cases, the antibody can bind to an antigen of a protein expressed on a cell in a tumor, including a tumor-specific protein. In some embodiments, the antibody is an antigen-binding fragment is a Fab, single V_{H} domain, a single chain variable fragment (scFv), a multivalent scFv, a bispecific scFv or an scFv-CH₃ dimer.

In some embodiments, the targeting molecule binds to an antigen or protein directly or indirectly. For example, in some embodiments, the targeting molecule is a second binding molecule that binds to a first binding molecule which is capable of binding to the antigen or protein. For example, the targeting molecule is a secondary antibody, which binds to a first binding molecule, e.g., a primary antibody, capable of binding the protein or antigen, e.g., a cell surface molecule or a cell surface receptor. Thus, in some embodiments, the dye is conjugated to a secondary antibody.

An “antibody” is a polypeptide ligand comprising at least a light chain and/or heavy chain immunoglobulin variable region that specifically recognizes and binds an epitope of an antigen. Generally, antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. The term antibody includes intact antibodies and antigen-binding antibody fragments that exhibit antigen-binding, such as Fab fragments, Fab′ fragments, F(ab)′₂ fragments, single chain Fv proteins (“scFv”), single domain antibodies (“sdAb”) and disulfide stabilized Fv proteins (“dsFv”). An scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as modified forms of immunoglobulins, chimeric antibodies, for example, humanized murine antibodies, and heteroconjugate antibodies, such as bispecific antibodies. See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes, or isotypes, which determine the functional activity of an antibody molecule: IgM, IgD, IgG, IgA and IgE.

Each heavy and light chain contains a constant region and a variable region, also known as “domains.” In combination, the heavy and the light chain variable regions generally specifically bind the antigen. Light and heavy chain variable regions may contain a “framework” region interrupted by three hypervariable regions, also called “complementarity-determining regions” or “CDRs.” The extent of the framework region and CDRs has been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are typically responsible for binding to an epitope of an antigen. The CDRs of each chain are typically referred to as CDR1, CDR2, and CDR3, numbered sequentially starting from the N-terminus, and are also generally identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities, such as different combining sites for different antigens, have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments. Other antibody fragments or multispecific antibodies formed from antibody fragments include a multivalent scFv, a bispecific scFv or an scFv-C_{H}3 dimer. Antibody fragments can be made by various techniques, including but not limited to proteolytic digestion of an intact antibody as well as production by recombinant host cells. In some embodiments, the targeting molecule is an antibody or an antigen-binding fragment that is a Fab, single V_{H} domain, a single chain variable fragment (scFv), a multivalent scFv, a bispecific scFv or an scFv-C_{H}3 dimer.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs, which generally confer antigen binding, from another species, such as a murine antibody that specifically binds mesothelin.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In some embodiments, the CDRs are from the donor immunoglobulin in a humanized immunoglobulin. Constant regions need not be present, but if they are, they may be substantially identical to human immunoglobulin constant regions, such as at least about 85-90%, such as about 95% or more identical. Hence, parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

A “human” antibody (also called a “fully human” antibody) is an antibody that includes human framework regions and CDRs from a human immunoglobulin. In some embodiments, the framework and the CDRs are from the same originating human heavy and/or light chain amino acid sequence. However, frameworks from one human antibody can be engineered to include CDRs from a different human antibody. Parts of a human immunoglobulin may be substantially identical to corresponding parts of natural human immunoglobulin sequences.

“Specifically binds” refers to the ability of a molecule, such as an antibody or antigen-binding fragment, to specifically bind an antigen, such as a tumor-specific antigen, relative to binding to unrelated proteins, such as non-tumor proteins, for example β-actin. In some embodiments, a molecule, such as an antibody or fragment, including a molecule, such as an antibody or fragment, attached to a phthalocyanine dye molecule and a therapeutic agent molecule, specifically binds to a target, such as a cell surface molecule, with a binding constant that is at least 10³ M⁻¹ greater, 10⁴ M⁻¹ greater or 10⁵ M⁻¹ greater than a binding constant for other molecules in a sample or subject. In some embodiments, a molecule, such as an antibody or fragments thereof, has an equilibrium association constant (K_{A}) of greater than or equal to about 10⁶M⁻¹, greater than or equal to about 10⁷M⁻¹, greater than or equal to about 10⁸M⁻¹, or greater than or equal to about 10⁹ M⁻¹ . 10¹⁰ M⁻¹, 10¹¹ M⁻¹ or 10¹² M⁻¹. Antibodies also can be characterized by an equilibrium dissociation constant (K_{D}) of 10⁻⁶M, 10⁻⁷ M, 10⁻⁸M, 10⁻¹⁰ M, 10⁻¹¹M or 10⁻¹²M or lower. In some embodiments, an equilibrium dissociation constant (K_{D}) can be 1 nM or less. Affinity constants, such as K_{D} or K_{A}, can be estimated empirically or affinities can be determined comparatively, e.g. by comparing the affinity of one antibody and another antibody for a particular antigen. For example, such affinities can be readily determined using techniques known in the art, such as, for example, by competitive ELISA (enzyme-linked immunosorbent assay) or using a surface-plasmon resonance device, such as the Biacore T100 (available from Biacore, Inc., Piscataway, N.J.), a radioimmunoassay using radiolabeled target antigen, or by another method known to the skilled artisan.

In some embodiments of the dual conjugates provided herein, the phthalocyanine dye (e.g., IR700) and/or the therapeutic agents are conjugated to an antibody or an antigen-binding antibody fragment. Exemplary antibodies to which the phthalocyanine dye (e.g., IR700) and/or the therapeutic agents can be conjugated to include, but are not limited to, cetuximab, panitumumab, zalutumumab, nimotuzumab, trastuzumab, Ado-trastuzumab emtansine, Tositumomab (Bexxar®), Rituximab (Rituxan, Mabthera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), Afatinib, Axitinib, Bosutinib, Cabozantinib, Ceritinib, Crizotinib, Dabrafenib, Dasatinib, Erlotinib, Everolimus, Ibrutinib, Imatinib, Lapatinib, Lenvatinib, Nilotinib, Olaparib, Palbociclib, Pazopanib, Pertuzumab, Ramucirumab, Regorafenib, Ruxolitinib, Sorafenib, Sunitinib, Temsirolimus, Trametinib, Vandetanib, Vemurafenib, Vismodegib, Basiliximab, Ipilimumab, Nivolumab, pembrolizumab, MPDL3280A, Pidilizumab (CT-011), MK-3475, BMS-936559, MPDL3280A, tremelimumab, IMP321, BMS-986016, LAG525, urelumab, PF-05082566, TRX518, MK-4166, dacetuzumab, lucatumumab, SEQ-CD40, CP-870, CP-893, MEDI6469, MEDI6383, MOXR0916, AMP-224, MSB0010718C, MEDI4736, PDR001, rHIgM12B7, Ulocuplumab, BKT140, Varlilumab (CDX-1127), ARGX-110, MGA271, lirilumab (BMS-986015, IPH2101), IPH2201, AGX-115, Emactuzumab, CC-90002 and 1VINRP1685A or an antibody-binding fragment thereof.

In some embodiments, the targeting molecule is a tissue-specific homing peptide. For example, in some embodiments, the homing polypeptide can contain the sequence of amino acids set forth in any of SEQ ID NOS: 1-52. In some embodiments, the targeting molecule is an RGD polypeptide, such as an iRGD polypeptide, a Lyp-1 polypeptide, a cripto-1 binding polypeptide, a somatostatin receptor binding polypeptide, or a prohibitin binding polypeptide, a NGR polypeptide, or an iNGR polypeptide.

In some embodiments, the targeting molecule is a viral particle, such as a virus-like particle, a viral-like nanoparticle, or a viral capsid. In some embodiments, the targeting molecule is a viral-like nanoparticle. In some embodiments, the viral-like nanoparticle is assembled from L1 capsid proteins. In some embodiments, the viral-like nanoparticle is assembled from a combination of L1 and L2 capsid proteins. In some embodiments, the targeting molecule can bind to and infect cells. In some embodiments, the targeting molecule is any one described in WO2015042325.

In some embodiments, a virus-like particle (VLP) refers to an organized capsid-like structure, such as roughly spherical or cylindrical in shape, that comprises self-assembling ordered arrays of L1 or L1 and L2 capsomers and does not include a viral genome. In some embodiments, virus-like particles are morphologically and antigenically similar to authentic virions, but they lack viral genetic material, such as viral nucleic acid, rendering the particles noninfectious. A VLP may be used to deliver to a recipient cell an agent, such as prophylactic agent, therapeutic agent or diagnostic agent, or an enclosed circular or linear DNA or RNA molecule.

In some embodiments, VLPs may have modified immunogenicity and/or antigenicity with respect to the wild type VLPs. The VLPs may, for example, be assembled from capsomers having a variant capsid protein with modified immunogenicity and/or antigenicity. In some embodiments, a variant capsid protein with modified immunogenicity and/or antigenicity is one that is modified naturally or synthetically, such as mutated, substituted, deleted, pegylated or inserted, at an amino acid to reduce or prevent recognition of the capsid protein by pre-existing, such as endogenous, viral serotype-specific antibodies. A variant capsid protein may be a human papillomavirus (HPV) L1 variant, a non-human papillomavirus L1 variant, or a papillomavirus L1 variant based on a combination of amino acids from different HPV serotypes.

In some embodiments, a VLP is a papilloma virus VLP. The VLP may be a human papilloma virus VLP, such as derived from a virus that can infect human, while in other embodiments, the VLP may be a non-human papilloma virus VLP. Examples of non-human VLPs include those derived from, without limitation, bovine papilloma viruses, murine papilloma viruses, cotton-rabbit papilloma viruses and macaque or rhesus papilloma virus particles. In some embodiments, the VLPs are bovine papilloma virus viral-like nanoparticles, such as type 1 viral-like nanoparticles, such as assembled from BPV L1 capsid proteins or a combination of BPV L1 and BPV L2 capsid proteins.

In some embodiments, a capsid protein refers to a protein monomer, several of which form a capsomer oligomer. In some embodiments, a capsomer refers to the basic oligomeric structural unit of a viral capsid, which is an outer covering of protein that protects the genetic material of a virus. Capsid proteins may include in some embodiments, papillomavirus L1 major capsid proteins and papillomavirus L2 minor capsid proteins. In some embodiments, the VLPs contain only L1 capsid proteins, while in other embodiments, the VLPs contain a mixture, or combination, of L1 and L2 capsid proteins.

In some embodiments, the percentage of L1 capsid proteins in a virus-like particle is greater than the percentage of L2 capsid proteins in the virus-like particle. For example, in some embodiments, the percentage of L1 capsid proteins in a virus-like particle is 80% to 100% of the total number of capsid proteins in the virus-like particle. In some embodiments, the percentage of L1 capsid proteins in a virus-like particle is at least or is about 80%, 85%, 90%, 91%, 92%, 93%, 94%, 95%, 96%, 97%, 98%, 99% or 100%. In some embodiments, the percentage of L2 capsid proteins in a virus-like particle is 1% to 25% of the total number of capsid proteins in the virus-like particle. For example, in some embodiments, the percentage of L2 capsid proteins in a virus-like particle is at least or about 1%, 2%, 3%, 4%, 5%, 6%, 7%, 8%, 9%, 10%, 11%, 12%, 13%, 14%, 15%, 16%, 17%, 18%, 19% or 20%.

In some embodiments, a virus-like particle contains 12 to 72 L2 proteins. In some embodiments, a virus-like particle contains 360 L1 proteins and 12 to 72 L2 proteins. In some embodiments, capsid proteins assemble into viral-like nanoparticles having a diameter of 20 to 60 nm. For example, capsid proteins may assemble into viral-like nanoparticles having a diameter of at least or about 20, 25, 30, 35, 40, 45, 50, 55 or 60 nm.

In some embodiments, the targeting molecule is not or does not include a nanocarrier. In some embodiments, the targeting molecule is not or does not include a virus-like particle, a nanoparticle, a liposome, a quantum dot, or a combination thereof.

In some embodiments, the targeting molecule is a DARPin (designed ankyrin repeat protein). Typically, DARPins are derived from natural ankyrin repeat proteins and bind to proteins including e.g., human receptors, cytokines, kinases, human proteases, viruses and membrane proteins (Molecular Partners AG Zurich Switzerland; see Chapter 5. “Designed Ankyrin Repeat Proteins (DARPins): From Research to Therapy”, Methods in Enzymology, vol 503: 101˜134 (2012); and “Efficient Selection of DARPins with Sub-nanomolar Affinities using SRP Phage Display”, J. Mol. Biol. (2008) 382, 1211-1227, the entire disclosures of which are hereby incorporated by reference. In some embodiments, the DARPin is an antibody mimetic protein having high specificity and high binding affinity to a target protein, which is prepared via genetic engineering. In some embodiments, DARPins have a structure comprising at least 2 ankyrin repeat motifs, for example, comprising at least 3, 4 or 5 ankyrin repeat motifs. The DARPins can have any suitable molecular weight depending on the number of repeat motifs. For example, the DARPins including 3, 4 or 5 ankyrin repeat motifs may have a molecular weight of about 10 kDa, about 14 kDa, or about 18 kDa, respectively.

In some embodiments, the DARPin includes a core part that provides structure and a target binding portion that resides outside of the core and binds to a target. In some embodiments, the structural core includes a conserved amino acid sequence and the target binding portion includes an amino acid sequence that differs depending on the target.

In some embodiments, such as when the targeting molecule is a polypeptide, such as an antibody or antigen-binding antibody fragment, the number of dye molecule per targeting molecule can be from or from about 2 to about 5, such as from or from about 2 to about 4, for example about 3 or 3. In some embodiments, for example where the targeting molecule is a nanoparticle, such as a virus-like particle (VLP), the number of dye molecules to targeting molecule can be from or from about 10 to about 1000, 10 to about 500, 50 to about 500, or 50 to about 1000. Thus, in some embodiments, the targeting molecule may contain about 10 to about 1000 dye molecules.

In some embodiments, such as where the targeting molecule is a VLP, more than one dye molecule may be conjugated to a single capsid protein. For example, a single capsid protein, such as LI or L2 capsid protein, may be conjugated to 1 to 5, such as 1, 2, 3, 4 or 5, dye molecules. Thus, more than one amino acid of a capsid protein may be conjugated to a dye molecule. In some embodiments, a single capsid protein may be conjugated to 1 to 2, 1 to 3, or 2 to 3 dye molecules. Thus, a dye molecule may be conjugated to 1, 2, 3, 4 or 5 different amino acids, such as lysine, arginine and/or histidine, or other amino acid, of a single capsid protein.

3. Therapeutic Agent

The provided dual conjugates contain a therapeutic agent, which can be linked, directly or indirectly, to one or both of the phthalocyanine dye or targeting molecule. In some embodiments, the therapeutic agent is one that is used in connection with treatment of a disease, disorder or condition, e.g. a tumor, in combination with PIT using the phthalocyanine-targeting molecule followed by irradiation. In some embodiments, the therapeutic agent can potentiate or enhance the effects of treatment of the PIT therapy by the phthalocyanine-targeting molecule (e.g. IR700-antibody). In some embodiments, the dual conjugate targets both the phthalocyanine-targeting molecule and the therapeutic agent to the site of the lesion, e.g., tumor. In some embodiments, the therapeutic agent can be released or delivered into the microenvironment of the lesion via cleavage of a releasable or cleavable moiety. In some embodiments, the therapeutic agent is an immune modulating agent or is an anti-cancer agent.

In some embodiments, the therapeutic agent is one that is used in therapy or treatment of a disease, disorder or condition. In some embodiments, the therapeutic agent can itself act also by binding to or targeting a protein on the surface of a cell or cells present in a microenvironment of a lesion that is associated with or present as a result of a disease, disorder or condition, e.g. a tumor. In some embodiments, the therapeutic agent is an antibody or antigen-binding fragment thereof that binds to an immunologic target, such as a cell surface receptor expressed on immune cells or cell surface proteins involved in immune modulation. In some aspects, the therapeutic agent is an immune modulating agent, such as an immune checkpoint inhibitor or a cytokine. In some aspects, the therapeutic agent itself can be an agent selected from those described in Section I.A.2 above. In some aspects, depending on the context and use of the dual conjugate, one type of molecule, e.g., a molecule that can specifically bind to or target another molecule and that also has therapeutic properties, may be considered either the targeting molecule component or therapeutic agent component within the dual conjugate.

a. Immune Modulating Agents

In some embodiments, the therapeutic agent is an immune modulating agent (also referred to herein as “immunomodulator”). In some aspects, immune modulating agents are substances that either, directly or indirectly, suppress or activate the body's immune response. For example, immune modulating agents that stimulate immune response to tumors and/or pathogens may be used in combination with photoimmunotherapy. In some embodiments of the dual conjugates provided herein, the therapeutic agent, e.g., immune modulating agent, is linked to the phthalocyanine dye or the targeting molecule via a releasable or cleavable linker. In some embodiments, the cleavage of the linker permits release of the therapeutic agent from the dual conjugate, thereby targeting the therapeutic agent, e.g., immune modulating agent, directly to the cells involved in a disease, disorder or condition and/or be released into the microenvironment of a lesion associated with the disease, disorder or condition, after the dual conjugate is localized or targeted to the site or microenvironment of the lesion. Thus, the dual conjugate can permit specific immune modulation at the site or microenvironment of the lesion and localized release and delivery of the therapeutic agent, e.g., immune modulating agent.

In some embodiments, the therapeutic agent can be any immune modulating agent that can stimulate, amplify and/or otherwise enhance an anti-tumor immune response, such as by inhibiting immunosuppressive signaling or enhancing immunostimulant signaling. In some embodiments, the immune modulating agent is a peptide, protein or is a small molecule. In some embodiments, the protein can be a fusion protein or a recombinant protein. In some embodiments, the immune modulating agent binds to an immunologic target, such as a cell surface receptor expressed on immune cells, such a T cells, B cells or antigen-presenting cells. For example, in some embodiments, the immune modulating agent is an antibody or antigen-binding antibody fragment, a fusion protein, a small molecule or a polypeptide.

In some embodiments, the immune modulating agent inhibits an immune checkpoint pathway. The immune system has multiple inhibitory pathways that are involved in maintaining self-tolerance and for modulating immune responses. It is known that tumors can use certain immune-checkpoint pathways as a major mechanism of immune resistance, particularly against T cells that are specific for tumor antigens (Pardoll, 2012, Nature Reviews Cancer 12:252-264). Because many such immune checkpoints are initiated by ligand-receptor interactions, they can be readily blocked by antibodies against the ligands and/or their receptors.

Therefore, therapy with antagonistic molecules blocking an immune checkpoint pathway, such as small molecules, nucleic acid inhibitors (e.g., RNAi) or antibody molecules, are becoming promising avenues of immunotherapy for cancer and other diseases. In contrast to the majority of anti-cancer agents, checkpoint inhibitors do not necessarily target tumor cells directly, but rather target lymphocyte receptors or their ligands in order to enhance the endogenous antitumor activity of the immune system. (Pardoll, 2012, Nature Reviews Cancer 12:252-264).

As used herein, the term “immune checkpoint inhibitor” refers to molecules that totally or partially reduce, inhibit, interfere with or modulate one or more checkpoint proteins. Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptor ligands. Illustrative immune checkpoint molecules that may be targeted for blocking or inhibition include, but are not limited to, CD25, PD-1 (CD279), PD-L1 (CD274, B7-H1), PD-L2 (CD273, B7-DC), CTLA-4, LAG3 (CD223), TIM3, 4-1BB (CD137), 4-1BBL (CD137L), GITR (TNFRSF18, AITR), CD40, CD40L, ICOS, ICOS-L, OX40 (CD134, TNFRSF4), OX40L, CXCR2, tumor associated antigens (TAA), B7-H3, B7-H4, BTLA, HVEM, GAL9, B7H3, B7H4, CD28, VISTA, CD27, CD30, STING, A2A adenosine receptor, KIR, 2B4 (belongs to the CD2 family of molecules and is expressed on all NK, γδ, and memory CD8+ (αβ) T cells), CD160 (also referred to as BY55), CGEN-15049. Immune checkpoint inhibitors include antibodies, or antigen binding fragments thereof, or other binding proteins, that bind to and block or inhibit the activity of one or more of CD25, PD-1, PD-L1, PD-L2, CTLA-4, LAG3, TIM3, 4-1BB, 4-1BBL, GITR, CD40, CD40L, ICOS, ICOS-L, OX40, OX40L, CXCR2, TAA, B7-H3, B7-H4, BTLA, HVEM, GAL9, CD28, VISTA, CD27, CD30, STING, A2A adenosine receptor, KIR, 2B4, CD160, and CGEN-15049. Illustrative immune checkpoint inhibitors include Tremelimumab (CTLA-4 blocking antibody), anti-OX40, PD-L1 monoclonal antibody (Anti-B7-H1; MEDI4736), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and Yervoy/ipilimumab (anti-CTLA-4 checkpoint inhibitor).

Programmed cell death 1 (PD1) is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll, 2012, Nature Reviews Cancer 12:252-264). The major role of PD1 is to limit the activity of T cells in peripheral tissues during inflammation in response to infection, as well as to limit autoimmunity (Pardoll, 2012, Nature Reviews Cancer 12:252-264). PD1 expression is induced in activated T cells and binding of PD1 to one of its endogenous ligands acts to inhibit T-cell activation by inhibiting stimulatory kinases (Pardoll, 2012, Nature Reviews Cancer 12:252-264). PD1 also acts to inhibit the TCR “stop signal” (Pardoll, 2012, Nature Reviews Cancer 12:252-264). PD1 is highly expressed on Treg cells and may increase their proliferation in the presence of ligand (Pardoll, 2012, Nature Reviews Cancer 12:252-264). Anti-PD 1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD1 antibodies include nivolumab (Opdivo by BMS), pembrolizumab (Keytruda by Merck), pidilizumab (CT-011 by Cure Tech), lambrolizumab (MK-3475 by Merck), and AMP-224 (Merck).

PD-L1 (also known as CD274 and B7-H1) and PD-L2 (also known as CD273 and B7-DC) are ligands for PD1, found on activated T cells, B cells, myeloid cells, macrophages, and some types of tumor cells. Anti-tumor therapies have focused on anti-PD-L1 antibodies. The complex of PD1 and PD-L1 inhibits proliferation of CD8+ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). Exemplary anti-PD-L1 antibodies include MDX-1105 (Medarex), MEDI4736 (Medimmune) MPDL3280A (Genentech), BMS-935559 (Bristol-Myers Squibb) and MSB0010718C.

Cytotoxic T-lymphocyte-associated antigen (CTLA-4), also known as CD152, is a co-inhibitory molecule that functions to regulate T-cell activation. CTLA-4 is a member of the immunoglobulin superfamily that is expressed exclusively on T-cells. CTLA-4 acts to inhibit T-cell activation and is reported to inhibit helper T-cell activity and enhance regulatory T-cell immunosuppressive activity (Pardoll, 2012, Nature Reviews Cancer 12:252-264). Although the precise mechanism of action of CTLA-4 remains under investigation, it has been suggested that it inhibits T cell activation by outcompeting CD28 in binding to CD80 and CD86, as well as actively delivering inhibitor signals to the T cell (Pardoll, 2012, Nature Reviews Cancer 12:252-264). Anti-CTLA-4 antibodies have been used in clinical trials for the treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTLA-4 is the kinetics of anti-tumor effect, with a lag period of up to 6 months after initial treatment required for physiologic response (Pardoll, 2012, Nature Reviews Cancer 12:252-264). In some cases, tumors may actually increase in size after treatment initiation, before a reduction is seen (Pardoll, 2012, Nature Reviews Cancer 12:252-264). Exemplary anti-CTLA-4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89). In some embodiments, the immune modulating agent is not an anti-CTLA-4 antibody.

Lymphocyte activation gene-3 (LAG-3), also known as CD223, is another immune checkpoint protein. LAG-3 has been associated with the inhibition of lymphocyte activity and in some cases the induction of lymphocyte anergyh. LAG-3 is expressed on various cells in the immune system including B cells, NK cells, and dendritic cells. LAG-3 is a natural ligand for the MHC class II receptor, which is substantially expressed on melanoma-infiltrating T cells including those endowed with potent immune-suppressive activity. An exemplary anti-LAG-3 antibodies is BMS-986016. IMP321 is a soluble version of the immune checkpoint molecule LAG-3, which activates dendritic cells, increasing antigen presentation.

T-cell immunoglobulin domain and mucin domain-3 (TIM-3), initially identified on activated Th1 cells, has been shown to be a negative regulator of the immune response. Blockade of TIM-3 promotes T-cell mediated anti-tumor immunity and has anti-tumor activity in a range of mouse tumor models. Combinations of TIM-3 blockade with other immunotherapeutic agents such as TSR-042, anti-CD137 antibodies and others, can be additive or synergistic in increasing anti-tumor effects. TIM-3 expression has been associated with a number of different tumor types including melanoma, NSCLC and renal cancer, and additionally, expression of intratumoral TIM-3 has been shown to correlate with poor prognosis across a range of tumor types including NSCLC, cervical, and gastric cancers. Blockade of TIM-3 is also of interest in promoting increased immunity to a number of chronic viral diseases. TIM-3 has also been shown to interact with a number of ligands including galectin-9, phosphatidylserine and HMGB1, although which of these, if any, are relevant in regulation of anti-tumor responses is not clear at present.

4-1BB, also known as CD137, is transmembrane glycoprotein belonging to the TNFR superfamily. 4-1BB receptors are present on activated T cells and B cells and monocytes. An exemplary anti-4-1BB antibody is urelumab (BMS-663513), which has potential immunostimulatory and antineoplastic activities.

Glucocorticoid-induced TNFR family related gene (GITR) is also a member of the TNFR superfamily. GITR is upregulated on activated T cells, which enhances the immune system. An exemplary anti-GITR antibody is TRX518.

Cluster of differentiation 40 (CD40) is also a member of the TNFR superfamily. CD40 is a costimulatory protein found on antigen-presenting cells and mediates a broad variety of immune and inflammatory responses. CD40 is also expressed on some malignancies, where it promotes proliferation. Exemplary anti-CD40 antibodies are dacetuzumab (SGN-40), lucatumumab (Novartis, antagonist), SEA-CD40 (Seattle Genetics), and CP-870,893.

Tumor necrosis factor receptor superfamily, member 4 (TNFRSF4), also known as OX40 and CD134, is another member of the TNFR superfamily. OX40 is not constitutively expressed on resting naïve T cells and acts as a secondary co-stimulatory immune checkpoint molecule. Exemplary anti-OX40 antibodies are MEDI6469 and MOXR0916 (RG7888, Genentech).

CXCR2 is a chemokine receptor that is expressed on myeloid-derived supressor cells (MDSCs). CXCR2s contribute to tumor immune escape. It has been shown that anti-CXCR2 monoclonal antibody therapy, enhanced an anti-PD1 antibody-induced anti-tumor immune response and anti-tumor efficacy.

In some embodiments, the immune-modulating agent is cytokine. In some embodiments, the immune modulating agent is a cytokine or is an agent that induces increased expression of a cytokine in the tumor microenvironment. By “cytokine” is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immune modulating agent is a cytokine and the cytokine is IL-4, TNF-α, GM-CSF or IL-2. In some embodiments, the cytokine can be a pro-inflammatory cytokine, e.g., PDGF, TGF-β, VEGF, tumor necrosis factor-α (TNF-α), and endothelin-1. In some embodiments, the cytokine can be an anti-inflammatory cytokine, e.g., IL-10. In some embodiments, the cytokine is an IL-12 or an IL-2.

In some embodiments, the immune modulating agent is selected from among GM-CSF, CpG-ODN (CpG oligodeoxynucleotides), lipopolysaccharide (LPS), monophosphoryl lipid A (MPL), alum, recombinant Leishmania polyprotein, imiquimod, MF59, poly I:C, poly A:U, type 1 IFN, Pam3Cys, Pam2Cys, complete freund's adjuvant (CFA), alpha-galactosylceramide, RC-529, MDF2β, Loxoribine, anti-CD40 agonist, SIRPa antagonist, AS04, AS03, Flagellin, Resiquimod, DAP (diaminopimelic acid), MDP (muramyl dipeptide) and CAF01(cationic adjuvant formulation-01). In some embodiments, the immune modulating agent is a Toll-like receptor (TLR) agonist, an adjuvant or a cytokine. In some embodiments, the immune modulating agent is a TLR agonist and the TLR agonist is TLR agonist is a TLR4 agonist, a TLR7 agonist, a TLR8 agonist, or a TLR9 agonist. In some embodiments, the TLR agonist is selected from among triacylated lipoprotein, diacylated lipopeptide, lipoteichoic acid, peptidoglycan, zymosan, Pam3CSK4, dsRNA, polyI:C, Poly G10, Poly G3, CpG, 3M003, flagellin, lipopolysaccharide (LPS) Leishmania homolog of eukaryotic ribosomal elongation and initiation factor 4a (LeIF), MEDI9197, SD-101, and imidazoquinoline TLR agonists.

In some embodiments, the immune modulating agent can contain one or more interleukins or other cytokines. For example, the interleukin can include leukocyte interleukin injection (Multikine), which is a combination of natural cytokines.

In some embodiments, the immune modulating agent is a Toll-like receptor (TLR) agonist. In some embodiments, such agonists can include a TLR4 agonist, a TLR8 agonist, or a TLR9 agonist. Such an agonist may be selected from peptidoglycan, polyI:C, CpG, 3M003, flagellin, and Leishmania homolog of eukaryotic ribosomal elongation and initiation factor 4a (LeIF).

In some embodiments, the immune modulating agent can be one that enhances the immunogenicity of tumor cells such as patupilone (epothilone B), epidermal-growth factor receptor (EGFR)-targeting monoclonal antibody 7A7.27, histone deacetylase inhibitors (e.g., vorinostat, romidepsin, panobinostat, belinostat, and entinostat), the n3-polyunsaturated fatty acid docosahexaenoic acid, proteasome inhibitors (e.g., bortezomib), shikonin (the major constituent of the root of Lithospermum erythrorhizon,) and oncolytic viruses, such as TVec (talimogene laherparepvec). In some embodiments, the immune modulating agent activates immunogenic cell death of the cancer or tumor, such as antrhacyclins (doxorubicin, mitoxantron), BK channel agonists, bortezomib, botrtezomib plus mitocycin C plus hTert-Ad, Cardiac glycosides plus non-ICD inducers, cyclophosphamide, GADD34/PP1 inhibitors plus mitomycin, LV-tSMAC, and oxaliplatin. In some embodiments, the immune modulating agent can be an epigenetic therapy, such as DNA methyltransferase inhibitors (e.g., Decitabine, 5-aza-2′-deoxycytidine).

For example, in some embodiments, the immune modulating agent can be a DNA methyltransferase inhibitor, which can regulate expression of tumor associated antigens (TAA). TAAs are antigenic substances produced in tumor cells which trigger an immune response. TAAs are often down-regulated by DNA methylation in tumors to escape the immune system. Reversal of DNA methylation restores TAA expression, increasing the immunogencity of tumor cells. For example, demethylating agents such as decitabine (5-aza-2′-deoxycytidine) can upregulate expression of TAAs in tumor cells and increase immune recognition of the cancerous cells. Photoimmunotherapy would further expose TAAs to the immune system by disrupting cells.

In some embodiments, the dual conjugates provided herein can contain one or more immune modulating agents. In some embodiments, the one or more immune modulating agents are the same or different. In some embodiments, the dual conjugates can contain two or more different immune modulating agents.

Exemplary immune modulating agents can include, but are not limited to, bevacizumab, cetuximab, panitumumab, zalutumumab, nimotuzumab, Tositumomab (Bexxar®), Rituximab (Rituxan, Mabthera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), Basiliximab, nivolumab, pembrolizumab, pidilizumab, MK-3475, BMS-936559, MPDL3280A, ipilimumab, tremelimumab, IMP321, BMS-986016, LAG525, urelumab, PF-05082566, TRX518, MK-4166, dacetuzumab, lucatumumab, SEA-CD40, CP-870, CP-893, MED16469, MEDI6383, MEDI4736, MOXR0916, AMP-224, PDR001, MSB0010718C, rHIgM12B7, Ulocuplumab, BKT140, Varlilumab (CDX-1127), ARGX-110, MGA271, lirilumab (BMS-986015, IPH2101), IPH2201, AGX-115, Emactuzumab, CC-90002 and 1VINRP1685A or is an antibody-binding fragment thereof. In some embodiments, the immune modulating agent is an antibody or antigen-binding antibody fragment thereof. Exemplary of such antibodies include, but are not limited to, Daclizumab (Zenapax), Bevacizumab (Avastin®), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), IPH2201, ARGX-115, Emactuzumab, CC-90002 and 1VINRP1685A or an antibody-binding fragment thereof.

In some embodiments, for example, if the treatment of the tumor with the dual conjugate followed by light irradiation increases the presence of immunosuppressive cells in the tumor or increases the expression of immunosuppressive markers at the tumor, the therapeutic agent in the dual conjugate can include a therapeutically effective amount of an immune modulating agent capable of reducing the amount or activity of immunosuppressive cells in the tumor or capable of blocking the activity of the immunosuppressive marker or reducing the activity of a tumor promoting cell in the tumor or capable of blocking the activity of the tumor promoting marker can be administered.

b. Anti-Cancer Agents

In some embodiments of the dual conjugates provided herein, the therapeutic agent is an anti-cancer agent. In some embodiments, an anti-cancer agent can include any agent whose use can reduce, arrest or prevent cancer in a subject. Optionally, an additional anti-cancer agent can be used in combination therapy with the dual conjugates provided herein, e.g., a dual conjugate that contains an immune modulating agent, for example to treat various cancers.

As described herein, PIT-induced cell killing of tumor cells by administration of one or more of the dual conjugates to a subject having a tumor in combination with irradiation can lead to increases in tumor permeability, such as increases in vascular permeability around the tumor space. It is believed herein that the increase in permeability can result in rapid leakage of systemically available molecules into the tumor space, thereby maximizing exposure of the tumor to such molecules. In such embodiments, following irradiation and PIT-induced killing of tumor cells, the anti-cancer agent available in the local microenvironment of the tumor by virtue of the targeting molecule binding to a cell surface molecule present in the tumor microenvironment (TME), the anti-cancer agent can be immediately taken up into the tumor space where the agent can provide a therapeutic effect.

In some embodiments of the dual conjugates provided herein, the therapeutic agent, e.g., anti-cancer agent, is linked to the phthalocyanine dye or the targeting molecule via a releasable or cleavable linker. In some embodiments, the cleavage of the linker permits release of the therapeutic agent from the dual conjugate, thereby targeting the therapeutic agent, e.g., anti-cancer agent, directly to the cells involved in a disease, disorder or condition and/or be released into the microenvironment of a lesion associated with the disease, disorder or condition, after the dual conjugate is localized or targeted to the site or microenvironment of the lesion. Thus, the dual conjugate can permit targeted delivery and/or release of the anti-cancer agent in the tumor microenvironment.

In contrast to combination therapy methods where a therapeutic agent is administered systemically and requires separate administration of the therapeutic agent(s), the dual conjugates provided herein permit rapid and effective delivery of the additional therapeutic agent, e.g., anti-cancer agent, to the site or microenvironment of the lesion, and reduce any lag time required in achieving a therapeutic effect because the anti-cancer agent is available for direct and immediate uptake into the tumor space. This can maximize therapeutic responses to the anti-cancer agent.

In some embodiments, the therapeutic agent contained in the dual conjugates provided herein that is an anti-cancer agent can refer to any agents, or compounds, used in anti-cancer treatment. These include any agents, when used alone or in combination with other compounds, that can alleviate, reduce, ameliorate, prevent, or place or maintain in a state of remission of clinical symptoms or diagnostic markers associated with tumors and cancer, and can be used in combinations and compositions provided herein. In some embodiments, the anti-cancer agent is one whose therapeutic effect is generally associated with penetration or delivery of the anti-cancer agent into the tumor microenvironment or tumor space.

In some embodiments, the anti-cancer agent is the anti-cancer agent is an alkylating agent, a platinum drug, an antimetabolite, an anti-tumor antibiotic, a topoisomerase inhibitor, a mitotic inhibitor, a corticosteroid, a proteasome inhibitor, a kinase inhibitor, a histone-deacetylase inhibitor, an anti-neoplastic agent, or an antibody or antigen-binding antibody fragment thereof or a combination thereof. In some embodiments, the anti-cancer agent is a peptide, protein or small molecule drug.

In some embodiments, the anti-cancer agent is 5-Fluorouracil/leukovorin, oxaliplatin, irinotecan, regorafenib, ziv-afibercept, capecitabine, cisplatin, paclitaxel, toptecan, carboplatin, gemcitabine, docetaxel, 5-FU, ifosfamide, mitomycin, pemetrexed, vinorelbine, carmustine wager, temozolomide, methotrexate, capacitabine, lapatinib, etoposide, dabrafenib, vemurafenib, liposomal cytarabine, cytarabine, interferon alpha, erlotinib, vincristine, cyclophosphamide, lomusine, procarbazine, sunitinib, somastostatin, doxorubicin, pegylated liposomal encapsulated doxorubicin, epirubicin, eribulin, albumin-bound paclitaxel, ixabepilone, cotrimoxazole, taxane, vinblastine, temsirolimus, temozolomide, bendamustine, oral etoposide, everolimus, octreotide, lanredtide, dacarbazine, mesna, pazopanib, eribulin, imatinib, regorafenib, sorafenib, nilotinib, dasantinib, celecoxib, tamoxifen, toremifene, dactinomycin, sirolimus, crizotinib, certinib, enzalutamide, abiraterone acetate, mitoxantrone, cabazitaxel, fluoropyrimidine, oxaliplatin, leucovorin, afatinib, ceritinib, gefitinib, cabozantinib, oxoliplatin or auroropyrimidine.

In some embodiments, the anti-cancer agent is an antibody or antigen-binding antibody fragment. In some embodiments, the anti-cancer agent can be any one or more of bevacizumab, cetuximab, panitumumab, ramucirumab, ipilimumab, rituximab, trastuzumab, ado-trastuzumab emtansine, pertuzumab, nivolumab, lapatinib, dabrafenib, vemurafenib, erlotinib, sunitinib, pazopanib, imatinib, regorafenib, sorafenib, nilotinib, dasantinib, celecoxib, crizotinib, certinib, afatinib, axitinib, bevacizumab, bosutinib, cabozantinib, afatinib, gefitinib, temsirolimus, everolimus, sirolimus, ibrutinib, imatinib, lenvatinib, olaparib, palbociclib, ruxolitinib, trametinib, vandetanib or vismodegib, or an antigen-binding antibody fragment thereof.

In some embodiments, the anti-cancer agent is an alkylating agent. Alkylating agents are compounds that directly damage DNA by forming covalent bonds with nucleic acids and inhibiting DNA synthesis. Exemplary alkylating agents include, but are not limited to, mechlorethamine, cyclophosphamide, ifosamide, melphalan, chlorambucil, busulfan, and thiotepa as well as nitrosurea alkylating agents such as carmustine and lomustine.

In some embodiments, the anti-cancer agent is a platinum drug. Platinum drugs bind to and cause crosslinking of DNA, which ultimately triggers apoptosis. Exemplary platinum drugs include, but are not limited to, cisplatin, carboplatin, oxaliplatin, satraplatin, picoplatin, nedaplatin, triplatin, and lipoplatin.

In some embodiments, the anti-cancer agent is an antimetabolite. Antimetabolites interfere with DNA and RNA growth by substituting for the normal building blocks of RNA and DNA. These agents damage cells during the S phase, when the cell's chromosomes are being copied. In some cases, antimetabolites can be used to treat leukemias, cancers of the breast, ovary, and the intestinal tract, as well as other types of cancer. Exemplary antimetabolites include, but are not limited to, 5-fluorouracil (5-FU), 6-mercaptopurine (6-MP), capecitabine) (Xeloda®), cytarabine (Ara-C®), floxuridine, fludarabine, gemcitabine (Gemzar®), hydroxyurea, methotrexate, and pemetrexed (Alimta®).

In some embodiments, the anti-cancer agent is an anti-tumor antibiotic. Anti-tumor antibiotics work by altering the DNA inside cancer cells to keep them from growing and multiplying. Anthracyclines are anti-tumor antibiotics that interfere with enzymes involved in DNA replication. These drugs generally work in all phases of the cell cycle. They can be widely used for a variety of cancers. Exemplary anthracyclines include, but are not limited to, daunorubicin, doxorubicin, epirubicin, and idarubicin. Other anti-tumor antibiotics include actinomycin-D, bleomycin, mitomycin-C, and mitoxantrone.

In some embodiments, the anti-cancer agent is a topoisomerase inhibitor. These drugs interfere with enzymes called topoisomerases, which help separate the strands of DNA so they can be copied during the S phase. Topoisomerase inhibitors can be used to treat certain leukemias, as well as lung, ovarian, gastrointestinal, and other cancers. Exemplary toposiomerase inhibitors include, but are not limited to, doxorubicin, topotecan, irinotecan (CPT-11), etoposide (VP-16), teniposide, and mitoxantrone.

In some embodiments, the anti-cancer agent is a mitotic inhibitor. Mitotic inhibitors are often plant alkaloids and other compounds derived from natural plant products. They work by stopping mitosis in the M phase of the cell cycle but, in some cases, can damage cells in all phases by keeping enzymes from making proteins needed for cell reproduction. Exemplary mitotic inhibitors include, but are not limited to, paclitaxel (Taxol®), docetaxel (Taxotere®), ixabepilone (Ixempra®), vinblastine (Velban®), vincristine (Oncovin®), vinorelbine (Navelbine®), and estramustine (Emcyt®).

In some embodiments, the anti-cancer agent is a corticosteroid. Corticosteroids, often simply called steroids, are natural hormones and hormone-like drugs that are useful in the treatment of many types of cancer. Corticosteroids can also be used before chemotherapy to help prevent allergic reactions as well as during and after chemotherapy to help prevent nausea and vomiting. Exemplary corticosteroids include, but are not limited to, prednisone, methylprednisolone (Solumedrol®), and dexamethasone (Decadron®).

In some embodiments, the anti-cancer agent is another type of chemotherapy drug, such as a proteosome inhibitor, a kinase inhibitor, or a histone-deacetylase inhibitor. In other embodiments, the anti-cancer agent is a biologic such as an antibody used in cancer therapy.

In some embodiments, the anti-cancer agent targets tumors associated with various cancers. The cancer can be any cancer located in the body of a subject, such as, but not limited to, cancers located at the head and neck, breast, liver, colon, ovary, prostate, pancreas, brain, cervix, bone, skin, eye, bladder, stomach, esophagus, peritoneum, or lung. For example, the anti-cancer agent can be used for the treatment of colon cancer, cervical cancer, cancer of the central nervous system, breast cancer, bladder cancer, anal carcinoma, head and neck cancer, ovarian cancer, endometrial cancer, small cell lung cancer, non-small cell lung carcinoma, neuroendocrine cancer, soft tissue carcinoma, penile cancer, prostate cancer, pancreatic cancer, gastric cancer, gall bladder cancer or espohageal cancer. In some cases, the cancer can be a cancer of the blood.

B. Linkage of Components

In some embodiments, the components of the dual conjugates provided herein, a phthalocyanine dye (e.g., IR700), a targeting molecule (e.g., antibody or antigen-binding fragment thereof) and a therapeutic agent (e.g., immune modulating agent or anti-cancer agent), are linked directly or indirectly, to the other components. In some embodiments, the dual conjugates provided herein contain one or more of each of the components, e.g., one or more phthalocyanine dye, one or more targeting molecule and one or more therapeutic agent, and each linkage, independently, can be direct or indirect, e.g., via a linker. In some embodiments, the linkage between the phthalocyanine dye and the targeting molecule and/or the therapeutic agent is covalent or non-covalent. In some embodiments, the linkage is indirect, e.g., via a linker, such as a cleavable linker.

In some embodiments, the phthalocyanine dye is linked directly or indirectly with the targeting molecule or the therapeutic agent. In some embodiments, the linkage between the phthalocyanine dye and the targeting molecule and/or the therapeutic agent is covalent or non-covalent. In some embodiments, the phthalocyanine dye is linked directly with the targeting molecule or the therapeutic agent.

In some embodiments, the therapeutic agent is linked directly or indirectly with the phthalocyanine dye or the targeting molecule. In some embodiments, the linkage between the therapeutic agent and the phthalocyanine dye or the targeting molecule is covalent or non-covalent. In some embodiments, the therapeutic agent is linked directly with the phthalocyanine dye or the targeting molecule.

In some embodiments, the targeting molecule is linked directly or indirectly with the phthalocyanine dye or the therapeutic agent. In some embodiments, the linkage between the therapeutic agent and the phthalocyanine dye or the targeting molecule is covalent or non-covalent. In some embodiments, the targeting molecule is linked directly with the phthalocyanine dye or the therapeutic agent. For example, in some embodiments, the targeting molecule is linked directly or indirectly to the phthalocyanine dye and/or the therapeutic agent. In some embodiments, the targeting molecule is linked directly or indirectly to one or more phthalocyanine dye molecules and one or more therapeutic agent molecules. In some embodiments, each of the linkage is independently direct or indirect.

In some embodiments, the targeting molecule, the phthalocyanine dye and/or the therapeutic agent are linked, directly or indirectly, to the other components via a covalent bond or a non-covalent interaction. In some embodiments, the covalent or non-covalent interactions or linkage is direct or indirect. In some embodiments, the attachment includes an indirect link, such as through a linker, binding moiety or domain or reactive group. In some embodiments, the linkage includes a direct interaction between the targeting molecule, the phthalocyanine dye and/or the therapeutic agent. In other embodiments, one or both or all of the targeting molecule, the phthalocyanine dye and/or the therapeutic agent are linked to one or more linkers, and the interaction is indirect, e.g., between a linker attached to one of the molecules and another molecule, or between two linkers, each attached to the targeting molecule and/or the phthalocyanine dye.

In some embodiments, the targeting molecule, the phthalocyanine dye and/or the therapeutic agent are non-covalently linked to or associated with the other components. For example, the phathalocyanine dye forms a complex with the targeting molecule and/or the therapeutic agent via a non-covalent interaction. In some embodiments, the phthalocyanine dye contains a moiety or domain capable of non-covalently interacting with an attachment group of the targeting molecule.

In some embodiments, in generating the dual conjugates provided herein, the components, e.g., the targeting molecule, the phthalocyanine dye and/or the therapeutic agent, can be incubated or bound to the other components to form a non-covalent interaction between the dye and the other components. In some examples, the non-covalent interaction between the targeting molecule, the phthalocyanine dye and/or the therapeutic agent include, for example, electrostatic interactions, van der Waals force, hydrophobic interactions, π-effects, ionic interactions, hydrogen bonding, halogen bonding and/or combinations thereof, or any interactions that depend on one or more of the forces. In some embodiments, the targeting molecule, the phthalocyanine dye and/or the therapeutic agent are linked using or using interactions that mimic non-covalent molecular interactions such as, for example, ligand-receptor interaction, antibody-antigen interaction, avidin-biotin interaction, streptavidin-biotin interaction, histidine-divalent metal ion interaction (e.g., Ni, Co, Cu, Fe), interactions between multimerization (e.g., dimerization) domains, glutathione S-transferase (GST)-glutathione interaction and/or any combination thereof.

In some embodiments, a non-covalent interaction moiety or domain is attached to or is a part of the targeting molecule, the phthalocyanine dye and/or the therapeutic agent, and forms a non-covalent interaction, e.g. a complex, with the other components of the dual conjugate. For example, in some embodiments, the non-covalent interaction molecule or domain is attached to or is a part of the phthalocyanine dye molecule, and forms a non-covalent interaction e.g. a complex, with the targeting molecule and/or the therapeutic agent. In other embodiments, the non-covalent interaction molecule or domain is attached to or is a part of the targeting agent, and forms a non-covalent interaction e.g. a complex, with the phthalocyanine dye molecule and/or the therapeutic agent. In other embodiments, non-covalent interaction molecule or domain is attached to or is a part of the therapeutic agent, and forms a non-covalent interaction e.g. a complex, with the targeting molecule and/or the phthalocyanine dye molecule. In some embodiments, a targeting molecule conjugated to biotin or an analog thereof (e.g. antibody-biotin, such as a cetuximab-biotin) and the phthalocyanine dye and/or therapeutic agent conjugated to an avidin or analog thereof or a streptavidin or analog thereof, including monomeric forms thereof (e.g. monomeric avidin-IR700 or monomeric streptavidin-IR700; or monomeric avidin-therapeutic agent or monomeric streptavidin-therapeutic agent, such as monomeric avidin-IL-12 or monomeric streptavidin-IL-12) are incubated or contacted for producing the dual conjugate. By virtue of the non-covalent interaction between avidin, streptavidin or analogs thereof and biotin or analogs thereof, in some embodiments, the phthalocyanine dye and/or the therapeutic agent forms a non-covalent complex with the targeting molecule.

In some embodiments, the therapeutic agent is linked indirectly via a linker to the phthalocyanine dye or the targeting molecule. For example, the linker can be a peptide, a polypeptide, or a chemical linker. Any peptide linkers, polypeptide linkers and chemical linkers known in the art can be used in the dual conjugates provide herein. For example, the linker is a peptide linker, or a cleavable peptide linker. In some embodiments, the linker is a covalent linker, wherein the covalent linkage is linear or branched, cyclic or heterocyclic, saturated or unsaturated, having 1-60 atoms, such as selected from among C, N, P, O, and S. In some embodiments, the linkage, e.g., chemical linkage, may contain any combination of ether, thioether, amine, ester, carbamate, urea, thiourea, oxy or amide bonds. In some embodiments, the linkage, e.g., chemical linkage, may include single, double, triple or aromatic carbon-carbon bonds, phosphorus-oxygen, phosphorus-sulfur, nitrogen-nitrogen, nitrogen-oxygen, nitrogen-platinum bonds, or aromatic or heteroaromatic bonds.

For example, in some embodiments, the linker can be a linker that has a reactive or activatable group, which is able to form a bond between the linker and the component being linked to. In some embodiments, the phthalocyanine dye contains a linker, i.e., is a linker-phthalocyanine dye moiety. In some embodiments, the linker contains a reactive group.

In some embodiments, the therapeutic agent is linked to the phthalocyanine dye and/or the targeting molecule via a releasable or cleavable linker. In some embodiments, the linker is not cleavable. In some embodiments, the release or cleavage of the linker permits release of the therapeutic agent from the dual conjugate. Thus, the therapeutic agent can be targeted or delivered directly to the cells involved in a disease, disorder or condition and/or be released into the microenvironment of a lesion associated with the disease, disorder or condition, by virtue of the targeting molecule binding a cell surface molecule on a cell in a microenvironment of a lesion.

The term “releasable linker” or “cleavable linker” as used herein, refers to a linker that includes at least one bond that can be broken under physiological conditions (e.g., a pH-labile, acid-labile, oxidatively-labile, or enzyme-labile bond). Physiological conditions resulting in breaking of the chemical bond can include standard chemical hydrolysis reactions that occur, for example, at physiological pH, or as a result of specific conditions present in a particular microenvironment, e.g., microenvironment of a lesion, such as the tumor microenvironment (TME).

In some embodiments, the releasable linker or the cleavable linker is released or cleaved in the microenvironment of the lesion. In some embodiments, the lesion is associated with specific microenvironment or physiological conditions. For example, in some embodiments, the lesion is a tumor, and the releasable linker or the cleavable linker is released or cleaved in the tumor microenvironment (TME), for example, under acidic or hypoxic conditions.

A variety of exemplary linkers that can be used in the dual conjugates, compositions and methods provided herein include those described in WO2004-010957, U.S. Publication Nos. 20060074008, 20050238649, and 20060024317.

In some embodiments, the linker is cleavable by a cleaving agent that is present in the microenvironment of a lesion. The linker can be, e.g., a peptidyl linker that is cleaved by a peptidase or protease enzyme. For example, the releasable linker or the cleavable linker is released or cleaved by a matrix metalloproteinase (MMP) present in in the TME. In some embodiments, the cleavable linker comprises the sequence of amino acids Pro-Leu-Gly-Leu-Trp-Ala (set forth in SEQ ID NO: 53). In some embodiments, the linker is cleavable by a cleaving agent that is overexpressed in the microenvironment of a lesion. In some embodiments, exemplary linkers include peptidyl linkers that are at least two amino acids long or at least three amino acids long. Exemplary linkers include a Phe-Leu linker, a Gly-Phe-Leu-Gly linker (SEQ ID NO:54), a Val-Cit linker or a Phe-Lys linker (see, e.g., U.S. Pat. No. 6,214,345). Other examples of such linkers are described, e.g., in U.S. Pat. No. 6,214,345 and Lu et al., (2016) Int. J. Mol. Sci. 17(4):561. In some embodiments, the linker is a linker cleavable by an enzyme that is overexpressed in the tumor interstitium, such as β-glucuronidase. In some embodiments, the linker is a β-glucuronide linker.

In some embodiments, the releasable linker or the cleavable linker is released or cleaved in hypoxic conditions or acidic conditions. In some embodiments, the conditions in the TME are acidic or hypoxic. In some embodiments, the linker is acid-labile or cleavable in hypoxic conditions. In some embodiments, the cleavable linker is cleavable under acidic conditions, and the cleavable linker comprises one or more hydrazone, semicarbazone, thiosemicarbazone, cis-aconitic amide, orthoester, acetal, ketal, 4-(4′-acetylphenoxy) butanoic acid or thioether linkages. In some embodiments, the cleavable linker is cleavable under hypoxic conditions, and the linker comprises one or more disulfide linkages.

In other embodiments, the cleavable linker is pH-sensitive, i.e., sensitive to hydrolysis at certain pH values. Typically, the pH-sensitive linker hydrolyzable under acidic conditions, such as, for example, the microenvironment of a lesion. For example, an acid-labile linker that is hydrolyzable in acidic environments, e.g., a hydrazone, semicarbazone, thiosemicarbazone, cis-aconitic amide, orthoester, acetal or ketal linkage, can be used. In some embodiments, exemplary linkers include those described in e.g., U.S. Pat. Nos. 5,122,368; 5,824,805; 5,622,929; Dubowchik and Walker, 1999, Pharm. Therapeutics 83:67-123; Neville, et al., 1989, Biol. Chem. 264:14653-14661. Such linkers are relatively stable under neutral pH conditions, such as those in the blood, but are unstable in acidic conditions.

In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe, et al., 1987, Cancer Res. 47:5924-5931; Wawrzynczak, et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson, et al., 1995, Anticancer Res. 15:1387-93), a maleimidobenzoyl linker (Lau, et al., 1995, Bioorg-Med-Chem. 3(10):1299-1304), or a 3′-N-amide analog (Lau, et al., 1995, Bioorg-Med-Chem. 3(10):1305-12).

In some embodiments, the cleavable linker is cleavable by light irradiation. In some embodiments, the linker is photo-labile. In some embodiments, the linker comprises one or more photolabile phenacyl ester, photolabile hydrazine or photolabile o-nitrobenzyl linkages or photolabile quinoxaline with thioether.

### II. METHODS OF TREATMENT

In some embodiments, provided are methods for using and uses of the compositions containing a dual conjugate containing a phthalocyanine dye (e.g., IR-700), a targeting molecule (e.g., antibody or antigen-binding fragment thereof) and a therapeutic agent (e.g., an immune modulating agent or anti-cancer agent). In some embodiments, the dual conjugate is targeted to, or targets, a cell or pathogen associated with a disease, disorder or condition, such as via binding to a cell surface molecule or cell surface receptor expressed on a cell. Such methods and uses include therapeutic methods and uses, for example, involving administration of the dual conjugates to a subject having a disease, condition or disorder followed by irradiation to achieve photoimmunotherapy (PIT), thereby resulting in photolysis of such cells or pathogens to effect treatment of the disease, disorder or condition.

Also provided herein are methods of treatment, e.g., including administering any of the dual conjugate or compositions containing dual conjugate described herein, and irradiation to achieve PIT. In some aspects, also provided are methods of administering any of the dual conjugate or compositions containing dual conjugate described herein to a subject, such as a subject that has a disease, disorder or condition. In some aspects, also provided are uses of any of the dual conjugate or compositions containing dual conjugate described herein for treatment of a disease, disorder or condition. In some aspects, also provided are uses of any of the dual conjugate or compositions containing dual conjugate described herein for the manufacture of a medicament for the treatment of a disease, disorder or condition. In some aspects, also provided are any of the dual conjugate or compositions containing dual conjugate described herein, for use in treatment of a disease, disorder or condition, or for administration to a subject having a disease, disorder or condition. In some aspects, in the methods or uses of the dual conjugates or compositions provided herein includes irradiation to achieve PIT following administration of the dual conjugates or compositions.

In some embodiments, provided are methods for treating a lesion in a subject that involves a) administering to the subject a therapeutically effective amount of any of the dual conjugates provided herein, or any compositions or kits comprising any of the dual conjugates provided herein, and b) after administering the conjugate, irradiating the lesion at a wavelengths to induce phototoxic activity of the conjugate.

In some embodiments, the methods can be used for treating a lesion, such as a tumor or a cancer, whereby an administered dual conjugate is targeted to a cell associated with a tumor, thereby resulting in photolysis of such cell and, in some cases, resulting in treatment of the tumor, and delivery or release of the therapeutic agent to the site of the tumor. In some embodiments, the therapeutic agent can be released at the site of the lesion by virtue of cleavage of the releasable or cleavable linker. Uses include uses of the compositions in such methods and treatments, and uses of such compositions in the preparation of a medicament in order to carry out such therapeutic methods. In some embodiments, the methods and uses thereby treat the disease or condition or disorder, such as a tumor or cancer, in the subject.

In some embodiments, the methods include administration of the dual conjugate to the subject under conditions in which, generally, a cell targeted for killing is contacted with the dual conjugate. In some embodiments, the methods result in the binding of the targeting molecule (e.g., antibody) portion of the dual conjugate to a cell surface molecule associated with a tumor or cancer. After contacting or administering the dual conjugate, a local area of the subject containing the targeted cells, e.g., a cell or cells associated with a tumor, is exposed or irradiated with light absorbed by the dye, generally NIR light, thereby activating the dual conjugate to effect specific cell killing. In some embodiments, irradiation is performed at a wavelength of 600 nm to 850 nm at a dose of at least 1 J cm⁻² or at least 1 J/cm of fiber length. In some embodiments, the methods of administering a dual conjugate containing a phthalocyanine dye include methods similar to those described in U.S. Pat. No. 8,524,239 or U.S. publication No. US2014/0120119 for administering an antibody-IR700 conjugate.

A. Disease and Subjects to be Treated

In some embodiments, the dual conjugates or composition containing the dual conjugates is administered to a subject having a disease, condition or disorder. In some aspects, the disease, condition or disorder is associated with a lesion. In some embodiments, the lesion is a tumor. In some embodiments, the tumor is a cancer or a tumor that is associated with a cancer. In some embodiments, the cancer is a cancer of the head and neck, breast, liver, colon, ovary, prostate, pancreas, brain, cervix, bone, skin, lung, or blood. In some embodiments, cancer may include a malignant tumor characterized by abnormal or uncontrolled cell growth. Other features that may be associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. Metastatic disease may refer to cancer cells that have left the original tumor site and migrated to other parts of the body, for example via the bloodstream or lymph system. In some embodiments, a cell targeted by the disclosed methods is a cancer cell or an immune cell. In some embodiments, the cancer cell is a cancer stem cell. In some embodiments, a cell targeted by the disclosed methods is a cell that is a cancer cell, a tumor cell, an inflammatory cell, an immune cell, a neuron, a stem cell, a proliferating cell, or a cell in a hyperplasia. In some embodiments, the lesion is premalignant dysplasia, carcinoma in situ, neoplasm, hyperplasia tumor or a tumor that is associated with a cancer.

In some aspects, the target cell can be a cell that is not desired or whose growth is not desired, such as a tumor or cancer cell. In some embodiments, the cells can be growing in culture, or present in a mammal to be treated, such as a subject with cancer. Any target cell can be treated with the claimed methods. In some embodiments, the target cell expresses a cell surface molecule that is not substantially found on the surface of other normal cells. In some embodiments, an antibody can be selected that specifically binds to such protein, and a dual conjugate, such as any provided herein, may be generated for that protein. In some embodiments, the cell surface molecule is a tumor-specific protein. In some embodiments, the cell surface molecule is CD25, which can be used to target cells associated with undesired transplant rejection.

In some embodiments, the tumor cell is a cancer cell, such as a cell in a subject with cancer. Exemplary cells that can be targeted in the disclosed methods include cells of the following tumors: a liquid tumor such as a leukemia, including acute leukemia (such as acute lymphocytic leukemia, acute myelocytic leukemia, and myeloblastic, promyelocytic, myelomonocytic, monocytic and erythroleukemia), chronic leukemias (such as chronic myelocytic (granulocytic) leukemia and chronic lymphocytic leukemia), polycythemia vera, lymphoma, Hodgkin's disease, non-Hodgkin's lymphoma, multiple myeloma, Waldenstrom's macroglobulinemia, heavy chain disease). In some embodiments, the cell is a solid tumor cell, such as a sarcoma or carcinoma, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinomna, lung cancer, colorectal cancer, squamous cell carcinoma, basal cell carcinoma, adenocarcinoma, for example adenocarcinoma of the pancreas, colon, ovary, lung, breast, stomach, prostate, cervix, or esophagus, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, CNS tumors, such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma. In some embodiments, the cancer is a squamous cell carcinoma of the head and neck.

Exemplary tumors, such as cancers, that can be treated with the claimed methods include solid tumors, such as breast carcinomas, such as lobular and duct carcinomas, sarcomas, carcinomas of the lung, such as non-small cell carcinoma, large cell carcinoma, squamous carcinoma, and adenocarcinoma, mesothelioma of the lung, colorectal adenocarcinoma, stomach carcinoma, prostatic adenocarcinoma, ovarian carcinoma, such as serous cystadenocarcinoma and mucinous cystadenocarcinoma, ovarian germ cell tumors, testicular carcinomas and germ cell tumors, pancreatic adenocarcinoma, biliary adenocarcinoma, hepatocellular carcinoma, bladder carcinoma, including, for instance, transitional cell carcinoma, adenocarcinoma, and squamous carcinoma, renal cell adenocarcinoma, endometrial carcinomas, including, for instance, adenocarcinomas and mixed Mullerian tumors (carcinosarcomas), carcinomas of the endocervix, ectocervix, and vagina, such as adenocarcinoma and squamous carcinoma of each of same, tumors of the skin, such as squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma, esophageal carcinoma, carcinomas of the nasopharynx and oropharynx, including squamous carcinoma and adenocarcinomas of same, salivary gland carcinomas, brain and central nervous system tumors, including, for example, tumors of glial, neuronal, and meningeal origin, tumors of peripheral nerve, soft tissue sarcomas and sarcomas of bone and cartilage, and lymphatic tumors, including B-cell and T-cell malignant lymphoma. In some embodiments, the tumor is an adenocarcinoma.

The methods can also be used to treat liquid tumors, such as a lymphatic, white blood cell, or other type of leukemia. In some embodiments, the tumor treated is a tumor of the blood, such as a leukemia, for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia, lymphomas, such as Hodgkin's lymphoma and non-Hodgkin's lymphoma, and myelomas.

In some embodiments, the dual conjugate is targeted to a protein expressed on the surface of a lesion or on the surface of a cell present in the microenvironment of the lesion. For example, in some embodiments, the dual conjugate is targeted to a protein expressed on the surface of a cell in the tumor or on the surface of a cell in the microenvironment of the tumor. Exemplary of such cell surface molecules are any as described herein, including those described above.

In some embodiments, the protein on the cell surface of the target cell to be targeted is not present in significant amounts on other cells. For example, the cell surface molecule can be a receptor that is only found on the target cell type. In some embodiments, the protein expressed in the tumor, e.g., tumor-specific protein, can be HER1/EGFR, HER2/ERBB2, CD20, CD25 (IL-2Rα receptor), CD33, CD52, CD133, CD206, CEA, cancer antigen 125 (CA125), alpha-fetoprotein (AFP), Lewis Y, TAG72, vascular endothelial growth factor (VEGF), CD30, EpCAM, EphA2, Glypican-3, gpA33, mucins, CAIX, PSMA, folate-binding protein, gangliosides (such as GD2, GD3, GM1 and GM2), VEGF receptor (VEGFR), integrin αVβ3, integrin α5β1, ERBB3, MET, IGF1R, EPHA3, TRAILR1, TRAILR2, RANKL, FAP, tenascin, AFP, BCR complex, CD3, CD18, CD44, CTLA-4, gp72, HLA-DR 10 β, HLA-DR antigen, IgE, MUC-1, nuC242, PEM antigen, SK-1 antigen or PD-L1. In some embodiments, the tumor-specific protein is PD-L1, HER1/EGFR, HER2, CD20, CD25, CD33, CD52, prostate specific membrane antigen (PSMA), EpCAM, EphA2, CD206, CD44, CD133, Mesothelin, Glypican-3, or carcinoembryonic antigen (CEA). Other cell surface molecules include any as described above.

In some embodiments, the cell surface molecule is associated with a tumor, such as is a tumor-specific protein or tumor-specific antigen, such as members of the EGF receptor family (e.g., HER1, 2, 3, and 4) and cytokine receptors (e.g., CD20, CD25, IL-13R, CD5, CD52, etc.). In some embodiments, tumor specific proteins are those proteins that are unique to cancer cells or are much more abundant on them, as compared to other cells, such as normal cells. For example, HER2 is generally found in breast cancers, while HER1 is typically found in adenocarcinomas, which can be found in many organs, such as the pancreas, breast, prostate and colon.

Exemplary proteins associated with a tumor that can be found on a target cell, and to which targeting molecule, e.g. antibody or antibody fragment, specific for that protein can be used to formulate a dual conjugate containing a phthalocyanine dye, include but are not limited to: any of the various MAGEs (Melanoma-Associated Antigen E), including MAGE 1, MAGE 2, MAGE 3, and MAGE 4, any of the various tyrosinases, mutant ras, mutant p53, p97 melanoma antigen, human milk fat globule (HMFG) which may be associated with breast tumors, any of the various BAGEs (Human B melanoma-Associated Antigen E), including BAGE1 and BAGE2, any of the various GAGEs (G antigen), including GAGE1, GAGE2-6, various gangliosides, and CD25.

Other proteins associated with a tumor include the HPV 16/18 and E6/E7 antigens associated with cervical cancers, mucin (MUC 1)-KLH antigen which may be associated with breast carcinoma, CEA (carcinoembryonic antigen) which may be associated with colorectal cancer, gp100 which may be associated with for example melanoma, MARTI antigens which may be associated with melanoma, cancer antigen 125 (CA125, also known as mucin 16 or MUC16) which may be associated with ovarian and other cancers, alpha-fetoprotein (AFP) which may be associated with liver cancer, Lewis Y antigen which may be associated with colorectal, biliary, breast, small-cell lung, and other cancers, tumor-associated glycoprotein 72 (TAG72) which may be associated with adenocarcinomas, and the PSA antigen which may be associated with prostate cancer.

Other exemplary proteins associated with a tumor further include, but are not limited to, PMSA (prostate membrane specific antigen), which may be associated with solid tumor neovasculature, as well prostate cancer, HER-2 (human epidermal growth factor receptor 2) which may be associated with breast cancer, ovarian cancer, stomach cancer and uterine cancer, HER-1 which may be associated with lung cancer, anal cancer, and gliobastoma as well as adenocarcinomas, NY-ESO-1 which may be associated with melanoma, sarcomas, testicular carcinomas, and other cancers, hTERT (aka telomerase), proteinase 3, and Wilms tumor 1 (WT-1).

In some embodiments, the protein associated with a tumor is CD52 and may be associated with chronic lymphocytic leukemia, CD33 and may be associated with acute myelogenous leukemia, or CD20 and may be associated with Non-Hodgkin lymphoma.

In some embodiments, the lesion comprises neurons and the disease, disorder or condition is a neurological disorder, which optionally is pain. In some embodiments, the lesion comprises fat cells or adipocytes and the disease, disorder or condition involves excess fat. In some embodiments, the lesion comprises pathogen infected cells and the disease, disorder or condition is an infection. In some embodiments, the lesion comprises inflammatory cells and the disease, disorder or condition is an inflammation.

Thus, the disclosed methods can be used to treat any cancer that expresses a tumor-specific protein. In some embodiments, the tumor therapeutic is an antibody, an antigen binding fragment, a protein, a glycoprotein, a peptide, a polypeptide, a virus, a viral capsid, or a viral particle. In some embodiments, the tumor therapeutic is an antibody or an antigen binding fragment.

In some embodiments, the subject is a human or non-human mammal. In some embodiments, the subject is a human or veterinary subject, such as a mouse. In some embodiments, the subject is a mammal, such as a human, who has cancer, or is being treated for cancer. In some embodiments the disclosed methods are used to treat a subject who has a tumor, such as a tumor described herein. In some embodiments, the tumor has been previously treated, such as surgically or chemically removed, and the disclosed methods are used subsequently to kill any remaining undesired tumor cells that may remain in the subject.

The disclosed dual conjugates and methods can be used to treat any mammalian subject, such as a human, who has a tumor, such as a cancer, or has had such previously removed or treated. Subjects in need of the disclosed therapies can include human subjects having cancer, wherein the cancer cells express a tumor-specific protein on their surface that can specifically bind to the dual conjugate. For example, the disclosed dual conjugates and methods can be used as initial treatment for cancer either alone, or in combination with radiation or other chemotherapy. The disclosed methods can also be used in patients who have failed previous radiation or chemotherapy. Thus, in some embodiments, the subject is one who has received other therapies, but those other therapies have not provided a desired therapeutic response. The disclosed dual conjugates and methods can also be used in patients with localized and/or metastatic cancer.

In some embodiments, the method includes selecting a subject that will benefit from the disclosed therapies, such as selecting a subject having a tumor that expresses a cell surface molecule, such as a tumor-specific protein, that can specifically bind to a dual conjugate provided herein. For example, if the subject is determined to have a breast cancer that expresses HER1, the subject may be selected to be treated with a dual conjugate comprising anti-HER1-IR700-therapeutic agent, such as cetuximab-IR700-IL-2.

B. Dosage and Administration

In some aspects, the provided dual conjugates or the compositions provided herein containing a dual conjugate containing a phthalocyanine dye, a targeting molecule and a therapeutic agent, are administered in amounts that are sufficient to exert a therapeutically useful effect. Typically, the active agents are administered in an amount that does not result in undesirable side effects of the patient being treated, or that minimizes or reduces the observed side effects as compared to dosages and amounts required for single treatment with one of the above agents.

Methods of determining optimal dosages of a dual conjugate to a patient in need thereof, either alone or in combination with one or more other agents, may be determined by standard dose-response and toxicity studies that are well known in the art.

The amount of a therapeutic agent, such as the dual conjugate that is administered to a human or veterinary subject will vary depending upon a number of factors associated with that subject, for example the overall health of the subject. In some embodiments, an effective amount of the agent can be determined by varying the dosage of the product and measuring the resulting therapeutic response, such as the regression of a tumor. In some embodiments, effective amounts can be determined through various in vitro, in vivo or in situ immunoassays. In some embodiments, the disclosed agents can be administered in a single dose, or in several doses, as needed to obtain the desired response. In some embodiments, the effective amount is dependent on the source applied, the subject being treated, the severity and type of the condition being treated, and the manner of administration.

In some embodiments, a therapeutically effective amount is an amount of the dual conjugate or a composition containing the dual conjugate that alone, or together with an additional therapeutic agent, is sufficient to achieve a desired effect in a subject, or in a cell, being treated with the composition. The effective amount of the therapeutic agent, such as the dual conjugate can be dependent on several factors, including, but not limited to the subject or cells being treated, the particular therapeutic agent, and the manner of administration of the therapeutic composition. In some embodiments, a therapeutically effective amount or concentration is one that is sufficient to prevent advancement, such as metastasis, delay progression, or to cause regression of a disease, or which is capable of reducing symptoms caused by the disease, such as cancer. In some embodiments, a therapeutically effective amount or concentration is one that is sufficient to increase the survival time of a patient with a tumor.

In some embodiments, a therapeutically effective dose of the dual conjugate is between or between about 10 mg/m² and 5000 mg/m², such as between or between about 10 mg/m² and 3000 mg/m², 10 mg/m² and 1500 mg/m², 10 mg/m² and 750 mg/m², 10 mg/m² and 500 mg/m², 10 mg/m² and 250 mg/m², 10 mg/m² and 200 mg/m², 10 mg/m² and 100 mg/m², 10 mg/m² and 75 mg/m², 10 mg/m² and 50 mg/m², 10 mg/m² and 25 mg/m², 25 mg/m² and 5000 mg/m², 25 mg/m² and 3000 mg/m², 25 mg/m² and 1500 mg/m², 25 mg/m² and 750 mg/m², 25 mg/m² and 500 mg/m², 25 mg/m² and 250 mg/m², 25 mg/m² and 200 mg/m², 25 mg/m² and 100 mg/m², 25 mg/m² and 75 mg/m², 25 mg/m² and 50 mg/m², 50 mg/m² and 5000 mg/m², 50 mg/m² and 3000 mg/m², 50 mg/m² and 1500 mg/m², 50 mg/m² and 750 mg/m², 50 mg/m² and 500 mg/m², 50 mg/m² and 250 mg/m², 50 mg/m² and 200 mg/m², 50 mg/m² and 100 mg/m², 50 mg/m² and 75 mg/m², 75 mg/m² and 5000 mg/m², 75 mg/m² and 3000 mg/m², 75 mg/m² and 1500 mg/m², 75 mg/m² and 1000 mg/m², 75 mg/m² and 750 mg/m², 75 mg/m² and 500 mg/m², 75 mg/m² and 250 mg/m², 75 mg/m² and 225 mg/m², 75 mg/m² and 200 mg/m², 75 mg/m² and 100 mg/m², 100 mg/m² and 5000 mg/m², 100 mg/m² and 3000 mg/m², 100 mg/m² and 1500 mg/m², 100 mg/m² and 750 mg/m², 100 mg/m² and 500 mg/m², 100 mg/m² and 250 mg/m², 100 mg/m² and 200 mg/m², 100 mg/m² and 150 mg/m², 150 mg/m² and 5000 mg/m², 150 mg/m² and 3000 mg/m², 150 mg/m² and 1500 mg/m², 150 mg/m² and 750 mg/m², 150 mg/m² and 500 mg/m², 150 mg/m² and 250 mg/m², 150 mg/m² and 200 mg/m², 200 mg/m² and 5000 mg/m², 200 mg/m² and 3000 mg/m², 200 mg/m² and 1500 mg/m², 200 mg/m² and 750 mg/m², 200 mg/m² and 500 mg/m², 200 mg/m² and 250 mg/m², 250 mg/m² and 5000 mg/m², 250 mg/m² and 3000 mg/m², 250 mg/m² and 1500 mg/m², 250 mg/m² and 750 mg/m², 250 mg/m² and 500 mg/m², 500 mg/m² and 5000 mg/m², 500 mg/m² and 3000 mg/m², 500 mg/m² and 1500 mg/m², 500 mg/m² and 750 mg/m², 750 mg/m² and 5000 mg/m², 750 mg/m² and 3000 mg/m², 750 mg/m² and 1500 mg/m², 1500 mg/m² and 5000 mg/m², 1500 mg/m² and 3000 mg/m², and 3000 mg/m² and 5000 mg/m². In some embodiments, the therapeutically effective dose of the dual conjugate is no more than 10 mg/m², 50 mg/m², 75 mg/m², 100 mg/m², 150 mg/m², 200 mg/m², 225 mg/m², 250 mg/m², 300 mg/m², 400 mg/m², 500 mg/m², 600 mg/m², 700 mg/m², 800 mg/m², 900 mg/m², 1000 mg/m², 1250 mg/m², 1500 mg/m², 2000 mg/m², 2500 mg/m², 3000 mg/m², 3500 mg/m², 4000 mg/m², 4500 mg/m², or 5000 mg/m². In some embodiments, the dose is from or from about 50 mg/m² to about 5000 mg/m², from about 250 mg/m² to about 2500 mg/m², from about 750 mg/m² to about 1250 mg/m² or from about 100 mg/m² to about 1000 mg/m². In some embodiments, the dose is or is about 160 mg/m², 320 mg/m², 640 mg/m² or 1280 mg/m².

In some embodiments, a therapeutically effective dose of the dual conjugate is between or between about 0.25 mg/kg and 150 mg/kg, 0.25 mg/kg and 100 mg/kg, 0.25 mg/kg and 75 mg/kg, 0.25 mg/kg and 60 mg/kg, 0.25 mg/kg and 50 mg/kg, 0.25 mg/kg and 25 mg/kg, 0.25 mg/kg and 10 mg/kg, 0.25 mg/kg and 7.5 mg/kg, 0.25 mg/kg and 5.0 mg/kg, 0.25 mg/kg and 2.5 mg/kg, 0.25 mg/kg and 1.0 mg/kg, 0.25 mg/kg and 0.5 mg/kg, 0.50 mg/kg and 150 mg/kg, 0.50 mg/kg and 100 mg/kg, 0.50 mg/kg and 75 mg/kg, 0.50 mg/kg and 60 mg/kg, 0.50 mg/kg and 50 mg/kg, 0.50 mg/kg and 25 mg/kg, 0.50 mg/kg and 10 mg/kg, 0.50 mg/kg and 7.5 mg/kg, 0.50 mg/kg and 5.0 mg/kg, 0.50 mg/kg and 2.5 mg/kg, 0.50 mg/kg and 1.0 mg/kg, 1.0 mg/kg and 150 mg/kg, 1.0 mg/kg and 100 mg/kg, 1.0 mg/kg and 75 mg/kg, 1.0 mg/kg and 60 mg/kg, 1.0 mg/kg and 50 mg/kg, 1.0 mg/kg and 25 mg/kg, 1.0 mg/kg and 10 mg/kg, 1.0 mg/kg and 7.5 mg/kg, 1.0 mg/kg and 5.0 mg/kg, 1.0 mg/kg and 2.5 mg/kg, 2.5 mg/kg and 150 mg/kg, 2.5 mg/kg and 100 mg/kg, 2.5 mg/kg and 75 mg/kg, 2.5 mg/kg and 60 mg/kg, 2.5 mg/kg and 50 mg/kg, 2.5 mg/kg and 25 mg/kg, 2.5 mg/kg and 10 mg/kg, 2.5 mg/kg and 7.5 mg/kg, 2.5 mg/kg and 5.0 mg/kg, 5.0 mg/kg and 150 mg/kg, 5.0 mg/kg and 100 mg/kg, 5.0 mg/kg and 75 mg/kg, 5.0 mg/kg and 60 mg/kg, 5.0 mg/kg and 50 mg/kg, 5.0 mg/kg and 25 mg/kg, 5.0 mg/kg and 10 mg/kg, 5.0 mg/kg and 7.5 mg/kg, 7.5 mg/kg and 150 mg/kg, 7.5 mg/kg and 100 mg/kg, 7.5 mg/kg and 75 mg/kg, 7.5 mg/kg and 60 mg/kg, 7.5 mg/kg and 50 mg/kg, 7.5 mg/kg and 25 mg/kg, 7.5 mg/kg and 10 mg/kg, 10 mg/kg and 150 mg/kg, 10 mg/kg and 100 mg/kg, 10 mg/kg and 75 mg/kg, 10 mg/kg and 60 mg/kg, 10 mg/kg and 50 mg/kg, 10 mg/kg and 25 mg/kg, 25 mg/kg and 150 mg/kg, 25 mg/kg and 100 mg/kg, 25 mg/kg and 75 mg/kg, 25 mg/kg and 60 mg/kg, 25 mg/kg and 50 mg/kg, 50 mg/kg and 150 mg/kg, 50 mg/kg and 100 mg/kg, 50 mg/kg and 75 mg/kg, 50 mg/kg and 60 mg/kg, 60 mg/kg and 150 mg/kg, 60 mg/kg and 100 mg/kg, 60 mg/kg and 75 mg/kg, 75 mg/kg and 150 mg/kg, 75 mg/kg and 100 mg/kg, and 100 mg/kg and 150 mg/kg. In some embodiments, the therapeutically effective dose of the dual conjugate is no more than 0.25 mg/kg, 0.5 mg/kg, 1.0 mg/kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10.0 mg/kg, 15 mg/kg, 20 mg/kg, 25 mg/kg, 30 mg/kg, 40 mg/kg, 50 mg/kg, 60 mg/kg, 70 mg/kg, 75 mg/kg, 80 mg/kg, 90 mg/kg, 100 mg/kg, 125 mg/kg or 150 mg/kg.

In some embodiments, the therapeutically effective amount is at least or at least about 0.01 mg, 0.1 mg, 0.5 mg, 1 mg, 5 mg, 10 mg, 50 mg, 100 mg, 200 mg, 500 mg, 600 mg, 700 mg, 800 mg, 900 mg, 1000 mg, 2000 mg, 3000 mg or more.

In some embodiments, the methods include administering to a subject having a disease, disorder or condition a therapeutically effective amount of a dual conjugate. In some embodiments, the dual conjugate is targeted to a cell present in the microenvironment of a tumor, lesion or hyperplasia. In some embodiments, a therapeutically effective dose of the dual conjugate is administered intravenously. In some embodiments, a therapeutically effective dose of the dual conjugate is administered intratumorally.

In some embodiments, the dose of the dual conjugate is at least 10 μg/kg, such as at least 100 μg/kg, at least 500 μg/kg, or at least 500 μg/kg, for example 10 μg/kg to 1000 μg/kg, such as a dose of about 100 μg/kg, about 250 μg/kg, about 500 μg/kg, about 750 μg/kg, or about 1000 μg/kg, for example when administered intratumorally or intraperitoneally (IP). In some embodiments, the dose is at least 1 μg/ml, such as at least 500 μg/ml, such as between 20 μg/ml to 100 μg/ml, such as about 10 μg/ml, about 20 μg/ml, about 30 μg/ml, about 40 μg/ml, about 50 μg/ml, about 60 μg/ml, about 70 μg/ml, about 80 μg/ml, about 90 μg/ml or about 100 μg/ml, for example administered in topical solution.

In some embodiments, the therapeutically effective dose is a dose administered to a human. In some embodiments, the weight of an average human is 60 to 85 kg, such as about or approximately 75 kg.

In some embodiments, a therapeutically effective dose of the dual conjugate is less than 400 mg/m², less than 300 mg/m², less than 250 mg/m², less than 225 mg/m², less than 200 mg/m², less than 180 mg/m², less than 100 mg/m² or less than 50 mg/m². In some embodiments, a therapeutically effective dose of the dual conjugate is between or about between 50 mg/m² and 400 mg/m², 100 mg/m² and 300 mg/m², 100 mg/m² and 250 mg/m² or 100 mg/m² and 160 mg/m². In some embodiments, a therapeutically effective dose of the dual conjugate is between or between about 80 mg/m² and 240 mg/m², 80 mg/m² and 220 mg/m², 80 mg/m² and 200 mg/m², 80 mg/m² and 180 mg/m², 80 mg/m² and 160 mg/m², 80 mg/m² and 140 mg/m², 80 mg/m² and 120 mg/m², 80 mg/m² and 100 mg/m², 100 mg/m² and 240 mg/m², 100 mg/m² and 220 mg/m², 100 mg/m² and 200 mg/m², 100 mg/m² and 180 mg/m², 100 mg/m² and 160 mg/m², 100 mg/m² and 140 mg/m², 100 mg/m² and 120 mg/m², 120 mg/m² and 240 mg/m², 120 mg/m² and 220 mg/m², 120 mg/m² and 200 mg/m², 120 mg/m² and 180 mg/m², 120 mg/m² and 160 mg/m², 120 mg/m² and 140 mg/m², 140 mg/m² and 240 mg/m², 140 mg/m² and 220 mg/m², 140 mg/m² and 200 mg/m², 140 mg/m² and 180 mg/m², 140 mg/m² and 160 mg/m², 160 mg/m² and 240 mg/m², 160 mg/m² and 220 mg/m², 160 mg/m² and 200 mg/m², 160 mg/m² and 180 mg/m², 180 mg/m² and 240 mg/m², 180 mg/m² and 220 mg/m², 180 mg/m² and 200 mg/m², 200 mg/m² and 220 mg/m² or 200 mg/m² and 240 mg/m².

In some embodiments, a therapeutically effective dose of the dual conjugate is less than 12 mg/kg, less than 10 mg/kg, less than 8 mg/kg, less than 6 mg/kg, less than 4 mg/kg, less than 2 mg/kg or less than 1 mg/kg. In some embodiments, a therapeutically effective dose of the dual conjugate is between or between about 1 mg/kg and 12 mg/kg, 2 mg/kg and 10 mg/kg, 2 mg/kg and 6 mg/kg or 2 mg/kg and 4 mg/kg. In some embodiments, a therapeutically effective dose of the dual conjugate is between or between about 2.0 mg/kg and 6.5 mg/kg, 2.0 mg/kg and 6.0 mg/kg, 2.0 mg/kg and 5.0 mg/kg, 2.0 mg/kg and 4.0 mg/kg, 2.0 mg/kg and 3.0 mg/kg, 3.0 mg/kg and 6.5 mg/kg, 3.0 mg/kg and 6.0 mg/kg, 3.0 mg/kg and 5.0 mg·kg, 3.0 mg/kg and 4.0 mg/kg, 4.0 mg/kg and 6.5 mg/kg, 4.0 mg/kg and 6.0 mg/kg, 4.0 mg/kg and 5.0 mg/kg, 5.0 mg/kg and 6.5 mg/kg, 5.0 mg/kg and 6.0 mg/kg and 6.0 mg/kg and 6.5 mg/kg.

In some embodiments, the therapeutically effective amount is between about 75 mg and 500 mg, 75 mg and 400 mg, 75 mg and 400 mg, 75 mg and 300 mg, 75 mg and 200 mg, 75 mg and 150 mg, 150 mg and 500 mg, 150 mg and 400 mg, 150 mg and 300 mg, 150 mg and 200 mg, 200 mg and 500 mg, 200 mg and 400 mg, 200 mg and 300 mg, 300 mg and 500 mg, 300 mg and 400 mg or 400 mg and 500 mg.

In some embodiments, the therapeutically effective dose of the dual conjugate is for single dosage administration. In some embodiments, the therapeutically effective dose is administered as only a single injection or a single infusion in a dosage schedule or cycle, for example, is administered only one time in a dosage schedule or cycle. For example, in a dosing schedule or cycle, a subsequent dose of the dual conjugate is not administered. In some embodiments, the dosing schedule can be repeated. In some embodiments, the repeated dose, such as repeated single dose, is administered at a time in which the first dose has been cleared from the subject, which, in some cases, is a time at which there is no detectable systemic exposure of the dual conjugate. Thus, in some embodiments, the dosing of the dual conjugate is not administered to achieve a continuous systemic exposure of the dual conjugate, which is different than many existing therapies, including antibody therapies, in which repeating dosing in a dosing schedule or cycle is required to maintain continuous systemic exposure. In some embodiments, the dosing schedule or cycle is repeated once a week, every two weeks, once a month, twice a year, once a year or at a lesser frequency as needed.

In some embodiments, in any of the methods for using the dual conjugates or compositions provided herein, the dosing schedule is repeated, if residual lesion remains after a prior treatment with the dual conjugate. In some embodiments, the method additionally includes assessing the subject for the presence of a residual lesion and if residual lesion remains repeating the dosing schedule. In some embodiments, the dosing schedule is repeated if a residual lesion remains at a time that is more than or about or 1 week, 2 weeks, 3 weeks, 4 weeks, 2 months, 6 months or 1 year after initiation of the prior administration of the dual conjugate. In some embodiments, the dosing schedule is repeated if a residual lesion remains at or about 4 weeks after initiation of the prior administration of the dual conjugate.

One skilled in the art will recognize that higher or lower dosages of the dual conjugate can also be used, for example depending on the particular agent. In some embodiments, dosages, such as daily dosages, are administered in one or more divided doses, such as 2, 3, or 4 doses, or in a single formulation. The dual conjugate can be administered alone, in the presence of a pharmaceutically acceptable carrier, or in the presence of other therapeutic agents, such as an immune-modulating agent, anti-cancer agent or other anti-neoplastic agents.

In some embodiments, the dual conjugate may be administered either systemically or locally to the organ or tissue to be treated. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes. In some embodiments, the dual conjugate is administered intravenously. In some embodiments, the dual conjugate is administered parenterally. In some embodiments, the dual conjugate is administered enterally. In some embodiments, the dual conjugate is administered by local injection. In some embodiments, the dual conjugate is administered as a topical application.

In some aspects, the provided dual conjugates or the compositions comprising the dual conjugate can be administered locally or systemically using any method known in the art, for example to subjects having a tumor, such as a cancer, or who has had a tumor previously removed, for example via surgery. Although specific examples are provided, one skilled in the art will appreciate that alternative methods of administration of the disclosed agents can be used. Such methods may include for example, the use of catheters or implantable pumps to provide continuous infusion over a period of several hours to several days into the subject in need of treatment.

In some embodiments, the dual conjugate is administered by parenteral means, including direct injection or infusion into a tumor, such as intratumorally. In some embodiments, the dual conjugate is administered to the tumor by applying the agent to the tumor, for example by bathing the tumor in a solution containing the agent, such as the dual conjugate, or by pouring the agent onto the tumor.

In addition, or alternatively, the disclosed compositions can be administered systemically, for example intravenously, intramuscularly, subcutaneously, intradermally, intraperitoneally, subcutaneously, or orally, to a subject having a tumor, such as cancer.

The dosages of the dual conjugate or compositions containing the dual conjugate to be administered to a subject are not subject to absolute limits, but will depend on the nature of the composition and its active ingredients and its unwanted side effects, such as immune response against the agent, the subject being treated, and the type of condition being treated and the manner of administration. Generally, the dose will be a therapeutically effective amount, such as an amount sufficient to achieve a desired biological effect, for example an amount that is effective to decrease the size, such as volume and/or weight, of the tumor, or attenuate further growth of the tumor, or decrease undesired symptoms of the tumor.

In some embodiments, the compositions used for administration of the agent, such as the dual conjugate contain an effective amount of the agent along with conventional pharmaceutical carriers and excipients appropriate for the type of administration contemplated. For example, in some embodiments, parenteral formulations may contain a sterile aqueous solution or suspension of the dual conjugate. In some embodiments, compositions for enteral administration may contain an effective amount of the dual conjugate in aqueous solution or suspension that may optionally include buffers, surfactants, thixotropic agents, and flavoring agents.

It is within the level of a skilled artisan to determine the appropriate dose, of administration of a dual conjugate comprising a particular therapeutic agent, e.g., immune modulating agent or anti-cancer agent, prior to performing irradiation to ensure sufficient systemic availability of the therapeutic agent. For example, in some embodiments, appropriate ratio of the component in the dual conjugates provided herein and corresponding doses, can be determined. In many cases, the pharmacokinetics of particular therapeutic agent, e.g., immune modulating agent or anti-cancer agents are known in the art, and may be considered in determining appropriate doses of the dual conjugate for administration. In some cases, pharmacokinetics can be assessed by measuring such parameters as the maximum (peak) plasma concentration (Cₘₐₓ), the peak time (i.e. when maximum plasma concentration occurs; Tₘₐₓ), the minimum plasma concentration (i.e. the minimum plasma concentration between doses of agent; Cₘᵢₙ), the elimination half-life (T_{1/2}) and area under the curve (i.e. the area under the curve generated by plotting time versus plasma concentration of the agent; AUC), following administration. The concentration of a particular agent, e.g., dual conjugate and/or therapeutic agent, in the plasma following administration, e.g., subcutaneous administration, can be measured using any method known in the art suitable for assessing concentrations of agents in samples of blood. For example, an immunoassay, such as an ELISA, or chromatography/mass spectrometry-based assays can be used.

C. Photoimmunotherapy

In some embodiments, provided are methods of treating a lesion comprising administering a therapeutically effective amount of any of the dual conjugates provided herein, or a composition or kit that contains the dual conjugates provided herein, and irradiating the lesion to effect photoimmunotherapy (PIT). Also provided are methods of treatment, method of administration and uses, e.g., uses in treatment or therapy or manufacture of a medicament, of the dual conjugates or composition or kit containing the dual conjugates, that includes irradiation to achieve PIT following administration of the dual conjugates or compositions. The PIT includes administration of a composition containing the dual conjugate followed by irradiation. In some embodiments, the methods provided herein include irradiating the tumor.

In some embodiments, after the cells are contacted with the dual conjugate, the cells are irradiated. Methods of irradiation are known in the art. As only cells expressing the cell surface molecule will typically be recognized by the targeting molecule, generally only those cells will have sufficient amounts of the dual conjugate bound to it. This may decrease the likelihood of undesired side effects, such as killing of normal cells, as the irradiation may only kill the cells to which the dual conjugate is bound, and generally not other cells.

In some embodiments, a cell is irradiated in vivo, for example, irradiating a subject who has previously been administered the dual conjugate or compositions containing the dual conjugate. In some embodiments, the subject is irradiated, for example, a tumor in the subject can be irradiated.

In some embodiments, the irradiation is effected after administration of the dual conjugate or compositions containing the dual conjugate. In some embodiments, the irradiation or illumination is carried out or effected between or between about 30 minutes and 96 hours after administering the dual conjugate, such as between 30 minutes and 48 hours, 30 minutes and 24 hours or 12 hours and 48 hours, such as generally at least 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or more after administering the dual conjugate. For example, the irradiation can be performed within about 24 hours after administering the dual conjugate. In some embodiments, irradiation is effected simultaneously with or near simultaneously with administration of the dual conjugate or composition containing the dual conjugate. In some embodiments, greater than 6 hours prior to irradiating or illuminating the tumor, the subject has been administered the dual conjugate comprising the targeting molecule, wherein the dual conjugate associates with the tumor. In some embodiments, the dual conjugate has been previously administered to the subject greater than or greater than about 12 hours, 24 hours, 26 hours, 48 hours, 72 hours or 96 hours prior to irradiating or illuminating the tumor.

In some embodiments, the cells, such as a tumor, are irradiated with a therapeutic dose of radiation at a wavelength within a range from or from about 400 nm to about 900 nm, such as from or from about 500 nm to about 900 nm, such as from or from about 600 nm to about 850 nm, such as from or from about 600 nm to about 740 nm, such as from about 660 nm to about 740 nm, from about 660 nm to about 710 nm, from about 660 nm to about 700 nm, from about 670 nm to about 690 nm, from about 680 nm to about 740 nm, or from about 690 nm to about 710 nm. In some embodiments, the cells, such as a tumor, are irradiated with a therapeutic dose of radiation at a wavelength of 600 nm to 850 nm, such as 660 nm to 740 nm. In some embodiments, the cells, such as a tumor, is irradiated at a wavelength of at least or about at least 600 nm, 620 nm, 640 nm, 660 nm, 680, nm, 700 nm, 720 nm or 740 nm, such as 690 ±50 nm, for example about 680 nm.

In some embodiments, the cells, such as a tumor, are irradiated at a dose of at least 1 J cm⁻², such as at least 10 J cm⁻², at least 30 J cm⁻², at least 50 J cm⁻², at least 100 J cm⁻², or at least 500 J cm⁻². In some embodiments, the dose of irradiation is from or from about 1 to about 1000 J cm⁻², from about 1 to about 500 J cm⁻², from about 5 to about 200 J cm⁻², from about 10 to about 100 J cm⁻², or from about 10 to about 50 J cm⁻². In some embodiments, the cells, such as a tumor, are irradiated at a dose of at least or at least about 2 J cm⁻², 5 J cm⁻², 10 J cm⁻², 25 J cm⁻², 50 J cm⁻², 75 J cm⁻², 100 J cm⁻², 150 J cm⁻², 200 J cm⁻², 300 J cm⁻², 400 J cm⁻², or 500 J cm⁻².

In some embodiments, the cells, such as a tumor, are irradiated or illuminated at a dose of at least 1 J/cm fiber length, such as at least 10 J/cm fiber length, at least 50 J/cm fiber length, at least 100 J/cm fiber length, at least 250 J/cm fiber length, or at least 500 J/cm fiber length. In some embodiments, the dose of irradiation is from or from about 1 to about 1000 J/cm fiber length, from about 1 to about 500 J/cm fiber length, from about 2 to about 500 J/cm fiber length, from about 50 to about 300 J/cm fiber length, from about 10 to about 100 J/cm fiber length, or from about 10 to about 50 J/cm fiber length. In some embodiments, the cells, such as a tumor, are irradiated at a dose of at least or at least about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

In some embodiments, the dose of irradiation or illumination in a human subject is from or from about 1 to about 400 J cm⁻², from about 2 to about 400 J cm⁻², from about 1 to about 300 J cm⁻², from about 10 to about 100 J cm⁻² or from about 10 to about 50 J cm⁻², from about such as is at least or at least about or is or within or within about or is or is about 10 J cm⁻², at least 30 J cm⁻², at least 50 J cm⁻², at least 100 J cm⁻². In some embodiments, the dose of irradiation in a human subject is from or from about 1 to 300 J/cm fiber length, 10 to 100 J/cm fiber length or 10 to 50 J/cm fiber length, such as is at least or at least about or is or within or within about or is or is about 10 J/cm fiber length, at least 30 J/cm fiber length, at least 50 J/cm fiber length, at least 100 J/cm fiber length. In some cases, it is found that a dose of irradiation in a human subject to achieve PIT can be less than is necessary for PIT in a mouse. For example, in some cases, 50 J/cm² (50 J cm⁻²) light dosimetry in an in vivo tumor mouse model is not effective for PIT, which is in contrast to what we can be observed in the clinic with human patients.

In some embodiments, the dose of irradiation following administration of the composition comprising the dual conjugate is at least 1 J cm⁻² or 1 J/cm of fiber length at a wavelength of 660-740 nm, for example, at least 10 J cm⁻² or 10 J/cm of fiber length at a wavelength of 660-740 nm, at least 50 J cm⁻² or 50 J/cm of fiber length at a wavelength of 660-740 nm, or at least 100 J cm⁻² or 100 J/cm of fiber length at a wavelength of 660-740 nm, for example 1.0 to 500 J cm⁻² or 1.0 to 500 J/cm of fiber length at a wavelength of 660-740 nm. In some embodiments, the wavelength is 660-710 nm. In some embodiments, the dose of irradiation following administration of the composition comprising the dual conjugate is at least 1.0 J cm⁻² or 1 J/cm of fiber length at a wavelength of 680 nm for example, at least 10 J cm⁻² or 10 J/cm of fiber length at a wavelength of 680 nm, at least 50 J cm⁻² or 50 J/cm of fiber length at a wavelength of 680 nm, or at least 100 J cm⁻² or 100 J/cm of fiber length at a wavelength of 680 nm, for example 1.0 to 500 J cm⁻² or 1.0 to 500 J/cm of fiber length at a wavelength of 680 nm. In some embodiments, multiple irradiations are performed, such as at least 2, at least 3, or at least 4 irradiations, such as 2, 3, 4, 5, 6, 7, 8, 9 or 10 separate administrations. Exemplary irradiation after administration of the dual conjugates or compositions provided herein include irradiating the tumor at a wavelength of 660 nm to 740 nm at a dose of at least 1 J cm⁻² or 1 J/cm of fiber length.

In some embodiments, a light or laser may be applied to the dye molecules, such as cells containing the dual conjugate, for from about 5 seconds to about 5 minutes. For example, in some embodiments, the light or laser is applied for or for about 5, 10, 15, 20, 25, 30, 35, 40, 45 50 or 55 seconds, or for within a range between any of two such values, to activate the dye molecules. In some embodiments, the light or laser is applied for or for about 1, 1.5, 2, 2.5, 3, 3.5, 4, 4.5 or 5 minutes, or more, or within a range between any two of such values. In some embodiments, the length of time a light or laser is applied can vary depending, for example, on the energy, such as wattage, of the light or laser. For example, lights or lasers with a lower wattage may be applied for a longer period of time in order to activate the dye molecule.

In some embodiments, a light or laser may be applied about 30 minutes to about 48 hours after administering the dual conjugate. For example, in some embodiments, the light or laser is applied at or at about 30, 35, 40, 45, 50 or 55 minutes after administering the dual conjugate, or within a range between any two of such values. In some embodiments, the light or laser is applied at or at about 1, 2, 3, 4, 5, 6, 7, 8, 9, 10, 11, 12, 13, 14, 15, 16, 17, 18, 19, 20, 21, 22, 23 or 24 hours after administering the dual conjugate, or is administered within a range between or between about any two of such values. In some embodiments, the light or laser is applied for between or between about 1 and 24 hours, such as between or between about 1 and 12 hours, 12 and 24 hours, 6 and 12 hours, or may be administered more than 24 following administration of the dual conjugate. In some embodiments, the light or laser is applied 36 or 48 hours after administering the dual conjugate.

In some embodiments, cells, or subjects, can be irradiated one or more times. Thus, irradiation can be completed in a single day, or may be done repeatedly on multiple days with the same or a different dosage, such as irradiation at least 2 different times, 3 different times, 4 different times 5 different times or 10 different times. In some embodiments, repeated irradiations may be done on the same day, on successive days, or every 1-3 days, every 3-7 days, every 1-2 weeks, every 2-4 weeks, every 1-2 months, or at even longer intervals.

In some embodiments, the dose or method of irradiation differs depending on the type or morphology of the tumor.

In some embodiments, the lesion is a tumor that is a superficial tumor. In some embodiments, the tumor is less than 10 mm thick. In some embodiments, irradiation is carried out using a microlens-tipped fiber for surface illumination. In some embodiments, the light irradiation dose is from or from about 5 J/cm² to about 200 J/cm².

In some embodiments, the provided methods include illuminating an superficial tumor in a subject with a microlens-tipped fiber for surface illumination with a light dose of from or from about 5 J/cm² to about 200 J/cm², wherein the tumor is associated with a phototoxic agent that includes a targeting molecule bound to a cell surface molecule of the tumor. In some embodiments, the light irradiation dose is or is about 50 J/cm².

In some embodiments, the lesion is a tumor that is an interstitial tumor. In some embodiments, the tumor is greater than 10 mm deep or is a subcutaneous tumor. In some embodiments, irradiation is carried out using cylindrical diffusing fibers that includes a diffuser length of 0.5 cm to 10 cm and spaced 1.8 ±0.2 cm apart. In some embodiments, the light irradiation dose is from or from about 20 J/cm fiber length to about 500 J/cm fiber length.

In some embodiments, the provided methods include illuminating an interstitial tumor in a subject with cylindrical diffusing fibers that includes a diffuser length of 0.5 cm to 10 cm and spaced 1.8±0.2 cm apart with a light dose of or about 100 J/cm fiber length or with a fluence rate of or about 400 mW/cm, wherein the tumor is associated with a phototoxic agent that includes a targeting molecule bound to a cell surface molecule of the tumor. In some embodiments, the tumor is greater than 10 mm deep or is a subcutaneous tumor. In some embodiments, the cylindrical diffusing fibers are placed in a catheter positioned in the tumor 1.8±0.2 cm apart. In some embodiments, the catheter is optically transparent.

D. Additional Therapy

In some embodiments, an additional therapy can be administered to the subject. In some embodiments, the additional therapy is an additional therapeutic agent or anti-cancer treatment. In some embodiments, the anti-cancer treatment comprises radiation therapy. In some embodiments, the additional therapy is an unconjugated version of the targeting molecule in the dual conjugates provided herein, and/or an unconjugated version of the therapeutic agent in the dual conjugates provided herein. In some embodiments, the additional therapy is a different therapy, e.g., radiation therapy or surgery, or administration of a different therapeutic than a component of the dual conjugate.

In some embodiments, prior to the irradiation, the subject can receive one or more other therapies as described herein. In some cases, the one or more other therapies can be administered prior to, during, or following administration of the dual conjugate. In some embodiments, the additional therapeutic agent can be administered during or simultaneously with administration of the dual conjugate. In some embodiments, the additional therapeutic agent can be administered after or following administration of the dual conjugate. For example, in some embodiments, the dual conjugate is administered prior to the one or more other therapies and the dual conjugate and one or more other therapies are each administered prior to irradiating the tumor. In some embodiments, the dual conjugate is administered subsequent to the one or more other therapies and the dual conjugate and one or more other therapies are each administered prior to irradiating the tumor. In some embodiments, the irradiation is carried out after administration of the additional therapeutic and the dual conjugate.

In some embodiments, the dual conjugate is administered prior to, simultaneously or subsequently to administration of additional therapy. In some embodiments, the dual conjugate is administered after administering the additional therapy but prior to irradiating the tumor to effect photoimmunotherapy (PIT). In some embodiments, the additional therapy is administered greater than or greater than about 30 minutes, 1 hour, 2 hours, 6 hours, 12 hours, 24 hours, 48 hours, 96 hours, one week, two weeks, three weeks or one month prior to irradiating the tumor. In some embodiments, at the time of or after the irradiation, the subject can receive one or more additional therapies. In some cases, the one or more additional therapies are thus also administered after administration of the dual conjugate. In some embodiments, the additional therapy is administered within or within about 0 to 24 hours of the irradiation, such as within or within about 5 minutes, 10 minutes, 30 minutes, 1 hour, 2 hours, 6 hours, 12 hours or 24 hours of the irradiation.

In some embodiments, the other or additional agent or agents administered, is an unconjugated targeting molecule or an unconjugated therapeutic agent. In some embodiments, the unconjugated targeting molecule is the same or substantially the same targeting molecule as the targeting molecule or the therapeutic agent of the dual conjugate. For example, in some embodiments, prior to administration of the dual conjugate, the targeting molecule, e.g., an unconjugated antibody that targets a protein or antigen, is administered to the subject. In some embodiments, the targeting molecule is administered up to 96 hours prior to administration of the dual conjugate. In some embodiments, the targeting molecule is administered at a dose within a range from or from about 10 mg/m² to about 500 mg/m². For example, the targeting molecule is cetuximab, and cetuximab is administered to the subject up to 96 hours prior to administration of the dual conjugate.

E. Exemplary Features

In some embodiments, a desired response of treatment according to the provided methods of treatment using the dual conjugate is to reduce or inhibit one or more symptoms associated with a lesion, e.g., a tumor or a cancer. In some embodiments, the one or more symptoms do not have to be completely eliminated for the composition to be effective.

For example, administration of a composition containing the dual conjugate followed by irradiation can decrease the size of a tumor, such as the volume or weight of a tumor, or metastasis of a tumor, for example by at least 20%, at least 30%, at least 40%, at least 50%, at least 60%, at least 70%, at least 80%, at least 90%, at least 95%, at least 98%, or at least 100%, as compared to the tumor size, volume, weight, or metastasis in the absence of the dual conjugate. In some embodiments, the difference in tumor size, volume, weight or metastasis is evident after at least 7 days, at least 10 days, at least 14 days, at least 30 days, at least 60 days, at least 90 days, or at least 120 days after the treatment(s). In some embodiments, tumor size and volume can be monitored by radiography, ultrasound imaging, necropsy, by use of calipers, by microCT or by ¹⁸F-FDG-PET. Tumor size also can be assessed visually. In particular examples, tumor size (diameter) can be measured directly using calipers.

In some embodiments, therapy using the provided dual conjugates and PIT (e.g. antibody-IR700-therapeutic agent/PIT), in accord with the methods herein can result in a tumor size, volume, weight or metastasis that is less than the tumor size, volume, weight or metastasis would be if it were treated with either the targeting molecule alone, the phthalocyanine dye-targeting molecule conjugate/PIT alone or the therapeutic agent alone, that is, there is a synergistic effect. For example, the therapy with the dual conjugates provided herein can decrease the size of a tumor, such as the volume or weight of a tumor, or metastasis of a tumor, for example by at least 1.2-fold, 1.5-fold, 2-fold, 3-fold, 4-fold, 5-fold, 6-fold, 7-fold, 8-fold, 9-fold, 10-fold or more as compared to the tumor size, volume, weight, or metastasis achieved in therapy methods involving monotherapy with the targeting molecule, in therapy methods involving monotherapy with PIT with a composition containing a corresponding phthalocyanine dye-targeting molecule conjugate followed by irradiation, or in therapy methods involving monotherapy with the therapeutic agent, e.g., immune modulating agent or anti-cancer agent.

In some embodiments, a desired response of treatment according to the provided methods is to kill a population of cells by a desired amount, for example by killing at least 20%, at least 50%, at least 60%, at least 70%, at least 80%, at least 90%, at least 95%, at least 98%, or at least 100% of the cells, as compared to cell killing in the absence of the dual conjugate and irradiation. In some embodiments, the difference in tumor cell killing is evident after at least 1 hour, at least 2 hours, at least 6 hours, at least 12 hours, at least 1 day, at least 2 days, at least 3 days, at least 4 days, at least 5 days, at least 6 days, at least 7 days, at least 10 days, at least 14 days or at least 30 days, after the treatment(s). In some embodiments, cell killing activity can be assessed by a variety of techniques known in the art including, but not limited to, cytotoxicity/cell viability assays that can be employed to measure cell necrosis and/or apoptosis, such as from a biopsy sample, following treatment(s), such as MTT (3-(4,5-dimethylthiazol-2-yl)-2,5-diphenyltetrazolium bromide) assays and other related tetrazolium salt based assays (e.g., XTT, MTS or WST), ATP assays, apoptosis assays (e.g., using labeled annexin V), such as TUNEL staining of infected cells, DNA fragmentation assays, DNA laddering assays, and cytochrome C release assays. In some cases, imaging methods can be used, such as positron emission tomography (PET), including FDG-PET, single photon emission CT (SPECT), diffusion weighted imaging (DWI), dynamic susceptibility-weighted contrast-enhanced (DSC) MR imaging or dynamic contrast-enhanced (DCE) MR imaging, CT perfusion methods, magnetic resonance spectroscopy (MRS) Such assays and methods are well known to one of skill in the art.

In some embodiments, the dual conjugates and methods of use provided herein can increase the killing of tumor cells, for example, by at least by at least 1.2-fold, 1.5-fold, 2-fold, 3-fold, 4-fold, 5-fold, 6-fold, 7-fold, 8-fold, 9-fold, 10-fold or more as compared to cell killing in therapy methods involving monotherapy with the targeting molecule, in therapy methods involving monotherapy with PIT with a composition containing a corresponding phthalocyanine dye-targeting molecule conjugate followed by irradiation, or in therapy methods involving monotherapy with the therapeutic agent, e.g., immune modulating agent or anti-cancer agent.

In some embodiments, a desired response is to increase the survival time of a patient with a tumor, or who has had a tumor recently removed, by a desired amount, for example to increase survival by at least 20%, at least 50%, at least 60%, at least 70%, at least 80%, at least 90%, at least 95%, at least 98%, or at least 100%, as compared to the survival time in the absence of the dual conjugate and irradiation. In some embodiments, increased survival is evident by an increase in one or more survival indicators from among duration of median progression-free survival, duration of response, median overall survival or other survival-related clinical endpoint. In some embodiments, the difference in survival is evident after at least 7 days, at least 10 days, at least 14 days, at least 30 days, at least 60 days, at least 90 days, at least 120 days, at least 6 months, at least 12 months, at least 24 months, or at least 5 years or more after the treatment(s). In some embodiments, the dual conjugates and methods of use provided herein, increases the duration of median progression-free survival, duration of response, median overall survival or other survival-related clinical endpoint by at least 3 months, at least 4 months, at least 5 months, at least 6 months, at least 7 months, at least 8 months, at least 9 months, at least 10 months, at least 11 months, at least 12 months, at least 18 months, at least 24 months, or at least 5 years or more compared to if a subject were treated with therapy methods involving monotherapy with the targeting molecule, therapy methods involving monotherapy with PIT with a composition containing a corresponding phthalocyanine dye-targeting molecule conjugate followed by irradiation, or therapy methods involving monotherapy with the therapeutic agent, e.g., immune modulating agent or anti-cancer agent.

In some embodiments, the dual conjugates and methods of use provided herein provided herein can increase the survival time of a treated subject, for example, by at least 1.2-fold, 1.5-fold, 2-fold, 3-fold, 4-fold, 5-fold, 6-fold, 7-fold, 8-fold, 9-fold, 10-fold or more as compared to the survival time in a subject receiving a monotherapy with the targeting molecule, a monotherapy with PIT with a composition containing a corresponding phthalocyanine dye-targeting molecule conjugate followed by irradiation, or a monotherapy with the therapeutic agent, e.g., immune modulating agent or anti-cancer agent. In some embodiments, the dual conjugates and methods of use provided herein, increases the duration of median progression-free survival, duration of response, median overall survival or other survival-related clinical endpoint by at least 3 months, at least 4 months, at least 5 months, at least 6 months, at least 7 months, at least 8 months, at least 9 months, at least 10 months, at least 11 months, at least 12 months, at least 18 months, at least 24 months, or at least 5 years or more compared to if it were treated with either a monotherapy with the targeting molecule, a monotherapy with PIT with a composition containing a corresponding phthalocyanine dye-targeting molecule conjugate followed by irradiation, or a monotherapy with the therapeutic agent, e.g., immune modulating agent or anti-cancer agent.

In one aspect, the response to treatment is characterized utilizing Response Evaluation Criteria in Solid Tumors (RECIST) criteria, which is the recommended guideline for assessment of tumor response by the National Cancer Institute (see Therasse et al., (2000) J. Natl. Cancer Inst. 92:205-216). In some embodiments, patients can be assessed for response to the therapy using RECIST criteria as outlined in the revised version 1.1 guidelines (RECIST 1.1, see Eisenhauer et al. (2009) European Journal of Cancer, 45:228-247). The criteria for objective status are required for protocols to assess solid tumor response. Representative criteria include the following: (1) Complete Response (CR), defined as complete disappearance of all measurable disease; no new lesions; no disease related symptoms; no evidence of non-measurable disease; (2) Partial Response (PR) defined as 30% decrease in the sum of the longest diameter of target lesions (e.g., tumor); (3) Progressive Disease (PD), defined as 20% increase in the sum of the longest diameter of target lesions or appearance of any new lesion; (4) Stable or No Response, defined as not qualifying for CR, PR, or PD (see Therasse et al., supra.)

In one aspect, the response to treatment is characterized utilizing the Choi response criteria based on computed tomography (CT), as described in Choi et al., (2007) J Clin Oncol. 25:1753-1759. The Choi criteria use tumor density as measured in Hounsfield unit (HU) by CT, whereas the RECIST criteria use one-dimensional tumor size (e.g., sum of the longest diameter of target lesions). Decreased density of tumors on CT is correlated with the development of tumor necrosis. For certain therapies that cause tumor necrosis without a substantial decrease in one-dimensional tumor size, the RECIST criteria may underestimate the response. Thus, for therapies that primarily result in tumor necrosis, the Choi criteria may be used to measure response (see also van der Veldt et al., (2010) Brit J Cancer 102:803-809; Weng et al., (2013) Oncol Letters 6:1707-1712). The criteria for objective status are required for protocols to assess solid tumor response. Representative criteria include the following: (1) Complete Response (CR), defined as disappearance of all target lesions and no new lesions; (2) Partial Response (PR) defined as a decrease in tumor size of >10% or decrease in tumor density (Hounsfield unit (HU)) of ≥15% on CT, no new lesions and no obvious progression of nonmeasurable disease; (3) Progressive Disease (PD), defined as an increase of tumor size of ≥10% and does not meet the PR criteria by tumor density (HU) or new lesions or new intratumoral nodules or increase in the size of the existing intratumoral nodules; and (4) Stable or No Response, defined as not qualifying for CR, PR, or PD and no symptomatic deterioration attributed to tumor progression.

In some embodiments, administration of the dual conjugates and use according to the methods provided herein, achieves a reduction in the size or volume of the tumor by at least 30%, at least 40%, at least 50%, at least 60%, at least 70%, at least 80% at least 90% or more within two weeks or one month of the irradiation compared to the size or volume of the tumor prior to the administration and irradiation.

In some embodiments, the objective response rate (ORR) can be determined, which is the percentage of subjects in which a CR or PR response is observed. ORR is commonly used to measure tumor response to treatment in oncology clinical trials.

In some embodiments, in a population of treated subjects, administration of the dual conjugates and use according to the methods provided herein, effects an improvement of a disorder- or cancer-related parameter compared to a similarly situated population of subjects treated with the targeting molecule (e.g., antibody or antigen-binding antibody fragment) that is not conjugated, the therapeutic agent that is not conjugated, or monotherapy with PIT with a composition containing a corresponding phthalocyanine dye-targeting molecule conjugate, wherein the parameter is selected from one or more of: a) objective response rate (ORR); b) progression free survival (PFS); c) overall survival (OS); d) reduction in toxicity; e) tumor response; of f) quality of life. In some embodiments, the parameter is improved by at least 10%, at least 20%, at least 30%, at least 40%, at least 50%, at least 60%, at least 70%, at least 80%, at least 90%, at least 100% or more.

In some embodiments, in a population of treated subjects, administration of the dual conjugates and use according to the methods provided herein, results in a PR in at least 50%, 60%, 70%, 80%, 90%, 95% or 100% of the treated subjects. In some embodiments, in a population of treated subjects, administration of the dual conjugates in accord with the provided methods results in a CR in at least 10%, 20%, 30%, 40%, 50%, 60%, 70%, 80%, 90%, 95% or 100% of the treated subjects.

In some embodiments, in a population of treated subjects, administration of the dual conjugates and use according to the methods provided herein, results in an ORR that is greater than about 13%, for example greater than about 15%, greater than about 20%, greater than about 30%, greater than about 40%, greater than about 50%, greater than about 60%, greater than about 70%, greater than about 80%, greater than about 95%, or greater than about 99%.

In some embodiments, the dual conjugates and methods of use provided herein, such as a dual conjugate that contains an immune modulating agent, can be used to stimulate an immune response in a cancer patient. Typically, immune responses may be detected by any of a variety of well-known parameters, including but not limited to in vivo or in vitro determination of: soluble immunoglobulins or antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death); an increase in cytotoxic T-cells, activated macrophages or natural killer cells; or any other criterion by which the presence of an immune response may be detected.

Procedures for performing these and similar assays are widely known and may be found, for example in Lefkovits (Immunology Methods Manual: The Comprehensive Sourcebook of Techniques, 1998; see also Current Protocols in Immunology; see also, e.g., Weir, Handbook of Experimental Immunology, 1986 Blackwell Scientific, Boston, Mass.; Mishell and Shigii (eds.) Selected Methods in Cellular Immunology, 1979 Freeman Publishing, San Francisco, Calif.; Green and Reed, 1998 Science 281:1309 and references cited therein.).

Detection of the proliferation of tumor-reactive T cells may be accomplished by a variety of known techniques. For example, T cell proliferation can be detected by measuring the rate of DNA synthesis, and tumor specificity can be determined by controlling the stimuli (such as, for example, a specific desired tumor- or a control antigen-pulsed antigen presenting cells) to which candidate tumor-reactive T cells are exposed. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca²⁺ flux, or dye uptake, such as 3-(4,5-dimethylthiazol-2-yl)-2,5-diphenyl-tetrazolium Alternatively, synthesis of lymphokines (such as interferon-gamma) can be measured or the relative number of T cells that can respond to a particular antigen may be quantified.

Detection of antibody production (e.g., tumor specific antibody production) may be achieved, for example, by assaying a sample (e.g., an immunoglobulin containing sample such as serum, plasma or blood) from a host treated with a composition according to the present invention using in vitro methodologies such as radioimmunoassay (MA), enzyme linked immunosorbent assays (ELISA), equilibrium dialysis or solid phase immunoblotting including Western blotting. In preferred embodiments ELISA assays may further include tumor antigen-capture immobilization of a target tumor antigen with a solid phase monoclonal antibody specific for the antigen, for example, to enhance the sensitivity of the assay. Elaboration of soluble mediators (e.g., cytokines, chemokines, lymphokines, prostaglandins, etc.) may also be readily determined by enzyme-linked immunosorbent assay (ELISA), for example, using methods, apparatus and reagents that are readily available from commercial sources (e.g., Sigma, St. Louis, Mo.; see also R & D Systems 2006 Catalog, R & D Systems, Minneapolis, Minn.).

Any number of other immunological parameters may be monitored using routine assays that are well known in the art. These may include, for example, antibody dependent cell-mediated cytotoxicity (ADCC) assays, secondary in vitro antibody responses, flow immunocytofluorimetric analysis of various peripheral blood or lymphoid mononuclear cell subpopulations using well established marker antigen systems, immunohistochemistry or other relevant assays. These and other assays may be found, for example, in Rose et al. (Eds.), Manual of Clinical Laboratory Immunology, 5th Ed., 1997 American Society of Microbiology, Washington, D.C.

### III. PHARMACEUTICAL COMPOSITIONS, KITS AND ARTICLES OF MANUFACTURE

Provided herein are pharmaceutical compositions containing a dual conjugate containing a phthalocyanine dye, a targeting molecule and a therapeutic agent. In some embodiments, the compositions can be used in methods of PIT as described herein. In some embodiments, the dual conjugate or compositions containing the dual conjugate can be packaged as an article of manufacture or a kit.

A. Compositions, Formulations and Dosage Forms

In some embodiments, the dual conjugates, e.g. dual conjugates, can be formulated in a pharmaceutically acceptable buffer, such as that containing a pharmaceutically acceptable carrier or vehicle. Generally, the pharmaceutically acceptable carriers or vehicles, such as those present in the pharmaceutically acceptable buffer, are can be any known in the art. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds. Pharmaceutically acceptable compositions generally are prepared in view of approvals for a regulatory agency or other agency prepared in accordance with generally recognized pharmacopeia for use in animals and in humans.

Pharmaceutical compositions can include carriers such as a diluent, adjuvant, excipient, or vehicle with which the compound is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

In some embodiments, pharmaceutical preparation can be in liquid form, for example, solutions, syrups or suspensions. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In some cases, pharmaceutical preparations can be presented in lyophilized form for reconstitution with water or other suitable vehicle before use.

In some embodiments, the nature of the pharmaceutically acceptable buffer, or carrier, depends on the particular mode of administration being employed. For instance, in some embodiments, parenteral formulations may comprise injectable fluids that include pharmaceutically and physiologically acceptable fluids such as water, physiological saline, balanced salt solutions, aqueous dextrose, or glycerol as a vehicle. In some embodiments, for solid compositions, for example powder, pill, tablet, or capsule forms, non-toxic solid carriers can include, for example, pharmaceutical grades of mannitol, lactose, starch, or magnesium stearate. In addition to biologically-neutral carriers, pharmaceutical compositions to be administered can in some embodiments contain minor amounts of non-toxic auxiliary substances, such as wetting or emulsifying agents, preservatives, and pH buffering agents, for example sodium acetate or sorbitan monolaurate.

The compounds can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compounds are formulated into pharmaceutical compositions using techniques and procedures well known in the art (see e.g., Ansel Introduction to Pharmaceutical Dosage Forms, Fourth Edition, 1985, 126). Generally, the mode of formulation is a function of the route of administration.

Compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. Other modes of administration also are contemplated. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant.

Parenteral administration, generally characterized by injection, either subcutaneously, intramuscularly, intratumorally, intravenously or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain an activator in the form of a solvent such as pH buffering agents, metal ion salts, or other such buffers. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U. S. Pat. No. 3,710,795) also is contemplated herein. The percentage of active compound contained in such parenteral compositions is highly dependent on the specific nature thereof, as well as the activity of the compound and the needs of the subject.

Injectables are designed for local and systemic administration. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate.

If administered intravenously, suitable carriers include physiological saline or phosphate buffered saline (PBS), and solutions containing thickening and solubilizing agents, such as glucose, polyethylene glycol, and polypropylene glycol and mixtures thereof.

The composition can be formulated for single dosage administration or for multiple dosage administration. The agents can be formulated for direct administration. The composition can be provided as a liquid or lyophilized formulation. Where the composition is provided in lyophilized form it can be reconstituted just prior to use by an appropriate buffer, for example, a sterile saline solution.

Compositions also can be administered with other biologically active agents, either sequentially, intermittently or in the same composition. Administration also can include controlled release systems including controlled release formulations and device controlled release, such as by means of a pump.

The most suitable route in any given case depends on a variety of factors, such as the nature of the disease, the progress of the disease, the severity of the disease and the particular composition which is used. For example, compositions are administered sytemically, for example, via intravenous administration. Subcutaneous methods also can be employed, although increased absorption times can be necessary to ensure equivalent bioavailability compared to intravenous methods.

Pharmaceutical compositions can be formulated in dosage forms appropriate for each route of administration. Pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Unit dosage forms, include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared. Pharmaceutical compositions can be formulated in dosage forms appropriate for each route of administration.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. All preparations for parenteral administration must be sterile, as is known and practiced in the art. In some embodiments, the compositions can be provided as a lyophilized powder, which can be reconstituted for administration as solutions, emulsions and other mixtures. They may also be reconstituted and formulated as solids or gels. The lyophilized powders can be prepared from any of the solutions described above.

The sterile, lyophilized powder can be prepared by dissolving a dual conjugate in a buffer solution. The buffer solution may contain an excipient which improves the stability of other pharmacological components of the powder or reconstituted solution, prepared from the powder.

In some embodiments, subsequent sterile filtration of the solution followed by lyophilization under standard conditions known to those of skill in the art provides the desired formulation. Briefly, the lyophilized powder is prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to ensure sterility, and apportioned into vials for lyophilization. Each vial can contain a single dosage (1 mg-1 g, generally 1-100 mg, such as 1-5 mg) or multiple dosages of the compound. The lyophilized powder can be stored under appropriate conditions, such as at about 4° C. to room temperature. Reconstitution of this lyophilized powder with a buffer solution provides a formulation for use in parenteral administration. The precise amount depends upon the indication treated and selected compound. Such amount can be empirically determined.

In some embodiments, the pH of the composition is between or between about 6 and 10, such as between or between about 6 and 8, between or between about 6.9 and 7.3, such as about pH 7.1. In some embodiments, the pH of the pharmaceutically acceptable buffer is at least or about 5, at least or about 6, at least or about 7, at least or about 8, at least or about 9 or at least or about 10, or is 7.1.

The compositions can be formulated for single dosage administration or for multiple dosage administration. The agents can be formulated for direct administration.

In some embodiments, the compositions provided herein are formulated in an amount for direct administration of the active compound, such as dual conjugate, in a range from or from about 0.01 mg to about 3000 mg, from about 0.01 mg to about 1000 mg, from about 0.01 mg to about 500 mg, from about 0.01 mg to about 100 mg, from about 0.01 mg to about 50 mg, from about 0.01 mg to about 10 mg, from about 0.01 mg to about 1 mg, from about 0.01 mg to about 0.1 mg, from about 0.1 mg to about 2000 mg, from about 0.1 mg to about 1000 mg, from about 0.1 mg to about 500 mg, from about 0.1 mg to about 100 mg, from about 0.1 mg to about 50 mg, from about 0.1 mg to about 10 mg, from about 0.1 mg to about 1 mg, from about 1 mg to about 2000 mg, from about 1 mg to about 1000 mg, from about 1 mg to about 500 mg, from about 1 mg to about 100 mg, from about 1 mg to about 10 mg, from about 10 mg to about 2000 mg, from about 10 mg to about 1000 mg, from about 10 mg to about 500 mg, from about 10 mg to about 100 mg, from about 100 mg to about 2000 mg, from about 100 mg to about 1000 mg, from about 100 mg to about 500 mg, from about 500 mg to about 2000 mg, from about 500 mg to about 1000 mg, and from about 1000 mg to about 3000 mg. In some embodiments, the volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL or more. For example, the composition is formulated for single dosage administration of an amount between or between about 100 mg and 500 mg, or between or between about 200 mg and 400 mg. In some embodiments, the composition is formulated for single dosage administration of an amount between or between about 500 mg and 1500 mg, 800 mg and 1200 mg or 1000 mg and 1500 mg. In some embodiments, the volume of the composition is between or between about 10 mL and 1000 mL or 50 mL and 500 mL; or the volume of the composition is at least 10 mL, 20 mL, 30 mL, 40 mL, 50 mL, 75 mL, 100 mL, 150 mL, 200 mL, 250 mL, 300 mL, 400 mL, 500 mL or 1000 mL.

In some embodiments, the entire vial contents of the formulations can be withdrawn for administration, or can be divided up into a plurality of dosages for multiple administrations. Upon withdrawal of an amount of drug for administration, the formulation can be further diluted if desired, such as diluted in water, saline (e.g., 0.9%) or other physiological solution.

In some embodiments, also provided are compositions and combinations containing an additional therapeutic agent for additional or combination therapy, which can be prepared in accord with known or standard formulation guidelines, such as described above. In some embodiments, the dual conjugates and the additional therapeutic agent are formulated as separate compositions. In some embodiments, the additional therapeutic agent is provided as a separate composition from the dual conjugate, and the two compositions are administered separately. The compositions can be formulated for parenteral delivery (i.e. for systemic delivery). For example, the compositions or combination of compositions are formulated for subcutaneous delivery or for intravenous delivery. The agents, such as the dual conjugate and/or additional therapeutic agent can be administered by different routes of administration.

B. Packaging and Articles of Manufacture

Also provided are articles of manufacture containing packaging materials, any pharmaceutical compositions or combinations provided herein, and a label that indicates that the compositions and combinations are to be used for treatment of cancers. Exemplary articles of manufacture are containers including single chamber and dual chamber containers. The containers include, but are not limited to, tubes, bottles and syringes. The containers can further include a needle for subcutaneous administration.

In some embodiments, the agents, e.g., dual conjugates, can be provided separately for packaging as articles of manufacture. In some embodiments, the article of manufacture contains pharmaceutical compositions containing the dual conjugates provided herein.

The articles of manufacture provided herein contain packaging materials. Packaging materials for use in packaging pharmaceutical products are well known to those of skill in the art. See, for example, U.S. Pat. Nos. 5,323,907, 5,052,558 and 5,033,252, each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The choice of package depends on the agents. In general, the packaging is non-reactive with the compositions contained therein.

The components can be packaged in the same of different container. For example, in some embodiments, the components are separately packaged in the same container. Generally, examples of such containers include those that have an enclosed, defined space that contains the dual conjugate, and a separate enclosed, defined space containing the other components or component such that the subsequent areas are separated to permit the components to be separately administered. Any container or other article of manufacture is contemplated, so long as the agents are separated from the other components prior to administration. For suitable embodiments see e.g., containers described in U.S. Pat. Nos. 3,539,794 and 5,171,081. In some embodiments, a plurality of containers is provided, each separately containing a dual conjugate, and an additional therapeutic agent. In such examples, the plurality of containers can be packaged together as a kit.

In some embodiments, a container containing the dual conjugate is contained in a light-protected container. In some embodiments, the container is a vial, such as a depyrogenated, glass vial. In some embodiments, the container, such as a vial, blocks light of a particular wavelength, such as a wavelength of light that is absorbed by the dye in the dual conjugates provided herein. In some embodiments, the dual conjugate is protected from light using containers that protect contents from light, or certain wavelengths or intensities of light. For example, in some embodiments, the container has a light transmittance of no more than 50%, no more than 40%, no more than 30%, no more than 20%, no more than 10%, no more than 5%, or no more than 1%. In some embodiments, the container protects from transmittance of light having a wavelength between or between about 500 nm and 725 nm, such as between or between about 650 nm and 725 nm, or does not transmit an intensity of light greater than 700 lux, 600 lux, 500 lux, 400 lux, 300 lux, 200 lux, or 100 lux. In some embodiments, the container is green, amber, translucent, opaque, or is wrapped in an opaque material, such as a foil, such as aluminum foil. In some embodiments, the container is sterile or depyrogenated.

In some embodiments, the article of manufacture contains pharmaceutical compositions containing the dual conjugates provided herein and an additional therapeutic agent. For example, in some embodiments, the compositions can be provided in combination with an additional therapeutic agent. In some embodiments, the dual conjugate and/or an additional therapeutic agent, can be packaged as an article of manufacture as separate compositions for administration together, sequentially or intermittently. The combinations can be packaged as a kit.

In some embodiments, the dual conjugates are provided in a plurality of sealable containers. For example, the containers can each individually comprising a fraction of a single administration dose of a composition containing the dual conjugates provided herein. In some embodiments, the combined amount of the dual conjugate in the plurality of sealable containers is between or between about 100 mg and 1500 mg, or 100 mg and 1200 mg. In some embodiments, the combined amount of the dual conjugate in the plurality of sealable container is between or between about 100 mg and 500 mg, between or between about 200 mg and 400 mg, between or between about 500 mg and 1500 mg, between or between about 800 mg and 1200 mg or between or between about 1000 mg and 1500 mg.

In some embodiments, the article of manufacture contains packaging material and a label or package insert containing instructions for combining the contents of the plurality of vials to prepare a single dosage formulation of the composition.

Selected compositions including articles of manufacture thereof also can be provided as kits. Kits can include a pharmaceutical composition described herein and an item for administration provided as an article of manufacture. The kit can, optionally, include instructions for application including dosages, dosing regimens and instructions for modes of administration and/or instructions for irradiation, e.g., according to any of the methods described herein for photoimmunotherapy (PIT). Kits also can include a pharmaceutical composition described herein and an item for diagnosis.

In some embodiments, the compositions used for administration of agents, such as the dual conjugate, contain an effective amount of each agent along with conventional pharmaceutical carriers and excipients appropriate for the type of administration contemplated.

In some embodiments, a single dosage amount of the agent, such as the dual conjugate, is comprised within a single container, such as a container in which the agent is stored. In some embodiments, a single dosage amount of the agent is comprised in a plurality of containers. Thus, in some embodiments, a plurality of containers, such as vials, are combined, in a container to be used for administration of the agent, such as an intravenous (IV) bag. In some embodiments, the container used for administration, such as IV bag, is prepared by opening one or a plurality of containers comprising the agent and placing the contents in the bag, such as until a desired dose of the agent for administration, e.g., infusion, is achieved. During the preparation of the administration container, such as IV bag, light precautions are taken to avoid exposure of the agent to light, such as the various light precautions described herein.

### IV. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “conjugate” refers to a polypeptide linked directly or indirectly to one or more other polypeptides or chemical moieties. Such conjugates include fusion proteins, those produced by chemical conjugates and those produced by any other methods. For example, a conjugate can refer to a phthalocyanine dye, such as an IR700 molecule, linked directly or indirectly to one or more other polypeptides or chemical moieties, such as to a targeting molecule that binds to or targets to a cell surface molecule.

As used herein, a composition refers to any mixture of two or more products, substances, or compounds, including cells. It may be a solution, a suspension, liquid, powder, a paste, aqueous, non-aqueous or any combination thereof.

As used herein, a “pharmaceutical composition” or “pharmaceutical formulation” refers to a preparation which is in such form as to permit the biological activity of an active ingredient contained therein to be effective, and which contains no additional components which are unacceptably toxic to a subject to which the formulation would be administered.

As used herein, a “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a derivative refers to a form of a drug that has undergone change or modification from a reference drug or agent, but still retains activity (e.g., exhibits increased or decreased activity) compared to the reference drug or agent. Typically a derivative form of a compound means that a side chain of the compound has been modified or changed.

As used herein, an analogue or analog of a drug or agent is a drug or agent that is related to a reference drug, but whose chemical and biological activities can be different. Typically, analogues exhibit similar activities to a reference drug or agent, but the activity can be increased or decreased or otherwise improved. Typically, an analogue form of a compound or drug means that the backbone core of the structure is modified or changed compared to a reference drug.

As used herein, a kit is a packaged combination that optionally includes other elements, such as additional reagents and instructions for use of the combination or elements thereof.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, an “article of manufacture” is a product that is made and, in some cases, that can be sold. In some embodiments, the term can refer to compositions contained in articles of packaging, such as in a container.

As used herein, “combination therapy” refers to a treatment in which a subject is given two or more therapeutic agents, such as at least two or at least three therapeutic agents, for treating a single disease. In some embodiments, each therapy can result in an independent pharmaceutical effect, and together can result in an additive or synergistic pharmaceutical effect.

As used herein, “disease” “disorder” or “condition” refer to a pathological condition in an organism resulting from cause or condition including, but not limited to, infections, acquired conditions, genetic conditions, and characterized by identifiable symptoms.

As used herein, “treating” a subject with a disease, disorder or or condition means that the subject's symptoms are partially or totally alleviated, or remain static following treatment. Hence treating encompasses prophylaxis, therapy and/or cure. Prophylaxis refers to prevention of a potential disease and/or a prevention of worsening of symptoms or progression of a disease.

As used herein, “treatment” means any manner in which the symptoms of a condition, disorder or disease or other indication, are ameliorated or otherwise beneficially altered.

As used herein, “therapeutic effect” means an effect resulting from treatment of a subject that alters, typically improves or ameliorates the symptoms of a disease, disorder or condition or that cures a disease, disorder or condition.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease, disorder or disorder.

As used herein, amelioration of the symptoms of a particular disease, disorder or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, the term “subject” refers to an animal, including a mammal, such as a human being.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### V. EXEMPLARY EMBODIMENTS

Among the provided embodiments are:

1. A dual conjugate, comprising a phthalocyanine dye, a targeting molecule and a therapeutic agent.

2. The dual conjugate of embodiment 1, wherein the phthalocyanine dye and therapeutic agent are each independently linked to the targeting molecule.

3. The dual conjugate of embodiment 1, wherein the targeting molecule and therapeutic agent are each independently linked to the phythalocyanine dye.

4. The dual conjugate of embodiment 1, wherein the phythalocyanine dye and the targeting molecule are each independently linked to the therapeutic agent.

5. The dual conjugate of embodiment 1, wherein the dual conjugate comprises the following components:

(phthalocyanine dye)n, (targeting molecule)q and (therapeutic agent)m, wherein:

n, q and m, which are selected independently, are at least 1.

6. The dual conjugate of embodiment 5, wherein n and q, which are selected independently, are 1 to 5.

7. The dual conjugate of embodiment 5, wherein n and m, which are selected independently, are 1 to 5.

8. The dual conjugate of embodiment 5, wherein q is 1, n is between 1 and 100, and m is between 1 and 5.

9. The dual conjugate of embodiment 5, wherein the ratio of n to q is from or from about 1 to about 1000, from or from about 1 to about 10 or from or from about 2 to about 5.

10. The dual conjugate of any of embodiments 1-9, wherein the targeting molecule is capable of binding a cell surface molecule on a cell in a microenvironment of a lesion.

11. The dual conjugate of any of embodiments 1-10, wherein the targeting molecule is linked directly with the phthalocyanine dye or the therapeutic agent.

12. The dual conjugate of any of embodiments 1-11, wherein the linkage between the targeting molecule and the phthalocyanine dye and/or the therapeutic agent is covalent or non-covalent.

13. The dual conjugate of any of embodiments 1-10, wherein the phthalocyanine dye is linked directly with the targeting molecule or the therapeutic agent.

14. The dual conjugate of any of embodiments 1-10 and 13, wherein the linkage between the phthalocyanine dye and the targeting molecule and/or the therapeutic agent is covalent or non-covalent.

15. The dual conjugate of any of embodiments 1-10, wherein the therapeutic agent is linked directly with the phthalocyanine dye or the targeting molecule.

16. The dual conjugate of any of embodiments 1-10 and 15, wherein the linkage between the therapeutic agent and the phthalocyanine dye or the targeting molecule is covalent or non-covalent.

17. The dual conjugate of any of embodiments 1-10, wherein the therapeutic agent is linked indirectly via a linker to the phthalocyanine dye or the targeting molecule.

18. The dual conjugate of any of embodiments 1-10, wherein the targeting molecule is linked indirectly via a linker to the phthalocyanine dye or the therapeutic agent.

19. The dual conjugate of any of embodiments 1-10, wherein the phthalocyanine dye is linked indirectly via a linker to the targeting molecule or the therapeutic agent.

20. The dual conjugate of any of embodiments 17-19, wherein the linker is a peptide or a polypeptide or is a chemical linker.

21. The dual conjugate of any of embodiments 17-20, wherein the linker is a releasable linker or a cleavable linker.

22. The dual conjugate of embodiment 21, wherein the releasable linker or the cleavable linker is released or cleaved in the microenvironment of the lesion.

23. The dual conjugate of embodiment 22, wherein the lesion is a tumor, and the releasable linker or the cleavable linker is released or cleaved in the tumor microenvironment (TME).

24. The dual conjugate of any of embodiments 21-23, wherein the releasable linker or the cleavable linker is released or cleaved by a matrix metalloproteinase (MMP) present in in the TME.

25. The dual conjugate of any of embodiments 21-24, wherein the cleavable linker comprises the sequence of amino acids PLGLWA.

26. The dual conjugate of any of embodiments 21-23, wherein the releasable linker or the cleavable linker is released or cleaved in hypoxic conditions or acidic conditions.

27. The dual conjugate of any of embodiments 21-23 and 26, wherein the cleavable linker is cleavable under acidic conditions, and the cleavable linker comprises one or more hydrazone, semicarbazone, thiosemicarbazone, cis-aconitic amide, orthoester, acetal, ketal or thioether linkages.

28. The dual conjugate of any of embodiments 21-23 and 26, wherein the cleavable linker is cleavable under hypoxic conditions, and the linker comprises one or more disulfide linkages.

29. The dual conjugate of any of embodiments 21-23, wherein the cleavable linker is cleavable by light irradiation, and the linker comprises one or more photolabile phenacyl ester, photolabile hydrazine or photolabile o-nitrobenzyl linkages or photolabile quinoxaline with thioether.

30. The dual conjugate of any of embodiments 1-29, wherein the therapeutic agent is an immune modulating agent and/or an anti-cancer agent.

31. The dual conjugate of embodiment 30, wherein the immune modulating agent is a cytokine or is an agent that induces increased expression of a cytokine in the microenvironment of the lesion.

32. The dual conjugate of embodiment 31, wherein the cytokine is selected from among IL-1, IL-1α, IL-2, IL-3, IL-4, IL-5, IL-6, IL-7, IL-8, IL-9, IL-10, IL-11, IL-12, IL-15, interferon (IFN)-α, IFN-β, IFN-γ, tumor necrosis factor (TNF)-α, TNF-β, human growth hormone, N-methionyl human growth hormone, parathyroid hormone, thyroxine, insulin, proinsulin, relaxin, prorelaxin, glycoprotein hormones such as follicle stimulating hormone (FSH), thyroid stimulating hormone (TSH), and luteinizing hormone (LH), hepatic growth factor, fibroblast growth factor (FGF), prolactin, placental lactogen, tumor necrosis factor-αand -β, mullerian-inhibiting substance, mouse gonadotropin-associated peptide, inhibin, activin, vascular endothelial growth factor (VEGF), integrin, thrombopoietin (TPO), nerve growth factors (NGF)-β, platelet-growth factor, transforming growth factor (TGF)-α, TGF-β, insulin-like growth factor (IGF)-1, IGF-2, erythropoietin (EPO), osteoinductive factors, macrophage-CSF (M-CSF), granulocyte-macrophage-CSF (GM-CSF), granulocyte-CSF (G-CSF), leukemia inhibitory factor (LIF), kit ligand (KL) and/or a portion and/or combination thereof.

33. The dual conjugate of any of embodiments 30-32, wherein the immune modulating agent is a cytokine and the cytokine is IL-2, IL-4, IL-12, IFN-γ, TNF-α or GM-CSF.

34. The dual conjugate of embodiment 30, wherein the immune modulating agent is an immune checkpoint inhibitor or an agonist.

35. The dual conjugate of embodiment 30 or embodiment 34, wherein the immune modulating agent specifically binds a molecule selected from among CD25, PD-1, PD-L1, PD-L2, CTLA-4, LAG-3, TIM-3, 4-1BB, GITR, CD40, CD40L, OX40, OX40L, CXCR2, B7-H3, B7-H4, BTLA, HVEM, CD28, VISTA, ICOS, ICOS-L, CD27, CD30, STING, and A2A adenosine receptor.

36. The dual conjugate of any of embodiments 30, 34 and 35, wherein the immune modulating agent is an antibody or an antigen-binding fragment thereof, a small molecule or a polypeptide.

37. The dual conjugate of any of embodiments 30 and 34-36, wherein the immune modulating agent is selected from among nivolumab, pembrolizumab, pidilizumab, MK-3475, BMS-936559, MPDL3280A, ipilimumab, tremelimumab, IMP31, BMS-986016, urelumab, TRX518, dacetuzumab, lucatumumab, SEQ-CD40, CP-870, CP-893, MED16469, MED14736, MOXR0916, AMP-224, and MSB001078C, or is an antigen-binding fragment thereof.

38. The dual conjugate of embodiment 30, wherein the anti-cancer agent is an alkylating agent, a platinum drug, an antimetabolite, an anti-tumor antibiotic, a topoisomerase inhibitor, a mitotic inhibitor, a corticosteroid, a proteasome inhibitor, a kinase inhibitor, a histone-deacetylase inhibitor, an anti-neoplastic agent, or a combination thereof.

39. The dual conjugate of embodiment 30 or embodiment 38, wherein the anti-cancer agent is an antibody or an antigen-binding fragment thereof, a small molecule or a polypeptide.

40. The dual conjugate of any of embodiments 30, 38 and 39, wherein the anti-cancer agent is selected from among 5-Fluorouracil/leukovorin, oxaliplatin, irinotecan, regorafenib, ziv-afibercept, capecitabine, cisplatin, paclitaxel, toptecan, carboplatin, gemcitabine, docetaxel, 5-FU, ifosfamide, mitomycin, pemetrexed, vinorelbine, carmustine wager, temozolomide, methotrexate, capacitabine, lapatinib, etoposide, dabrafenib, vemurafenib, liposomal cytarabine, cytarabine, interferon alpha, erlotinib, vincristine, cyclophosphamide, lomusine, procarbazine, sunitinib, somastostatin, doxorubicin, pegylated liposomal encapsulated doxorubicin, epirubicin, eribulin, albumin-bound paclitaxel, ixabepilone, cotrimoxazole, taxane, vinblastine, temsirolimus, temozolomide, bendamustine, oral etoposide, everolimus, octreotide, lanredtide, dacarbazine, mesna, pazopanib, eribulin, imatinib, regorafenib, sorafenib, nilotinib, dasantinib, celecoxib, tamoxifen, toremifene, dactinomycin, sirolimus, crizotinib, certinib, enzalutamide, abiraterone acetate, mitoxantrone, cabazitaxel, fluoropyrimidine, oxaliplatin, leucovorin, afatinib, ceritinib, gefitinib, cabozantinib, oxoliplatin and auroropyrimidine.

41. The dual conjugate of any of embodiments 30, 38 and 39, wherein the anti-cancer agent is selected from among bevacizumab, cetuximab, panitumumab, ramucirumab, ipilimumab, rituximab, trastuzumab, ado-trastuzumab emtansine, pertuzumab, nivolumab, lapatinib, dabrafenib, vemurafenib, erlotinib, sunitinib, pazopanib, imatinib, regorafenib, sorafenib, nilotinib, dasantinib, celecoxib, crizotinib, certinib, afatinib, axitinib, bevacizumab, bosutinib, cabozantinib, afatinib, gefitinib, temsirolimus, everolimus, sirolimus, ibrutinib, imatinib, lenvatinib, olaparib, palbociclib, ruxolitinib, trametinib, vandetanib or vismodegib, or an antigen-binding fragment thereof.

42. The dual conjugate of any of embodiments 1-41, wherein the phthalocyanine dye has a maximum absorption wavelength from or from about 600 nm to about 850 nm.

43. The dual conjugate of any of embodiments 1-42, wherein the phthalocyanine dye comprises the formula:

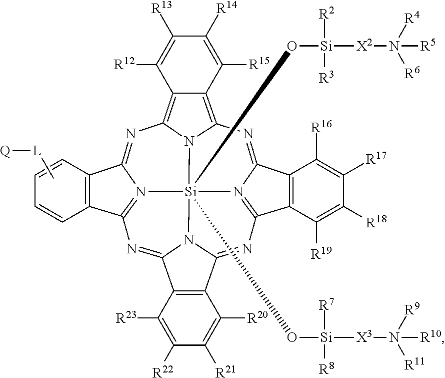

wherein:

L is a linker;

Q is a reactive group for attachment of the dye to the targeting molecule;

R², R³, R⁷, and R⁸ are each independently selected from optionally substituted alkyl and optionally substituted aryl;

R⁴, R⁵, R⁶, R⁹, R¹⁰, and R¹¹ are each independently selected from hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group;

R¹², R¹³, R¹⁴, R¹⁵, R¹⁶, R¹⁷, R¹⁸, R¹⁹, R²⁰, R²¹, R²² and R²³ are each independently selected from hydrogen, halogen, optionally substituted alkylthio, optionally substituted alkylamino and optionally substituted alkoxy; and

X² and X³ are each independently C₁-C₁₀ alkylene, optionally interrupted by a heteroatom.

44. The dual conjugate of any of embodiments 1-42, wherein the phthalocyanine dye comprises the formula:

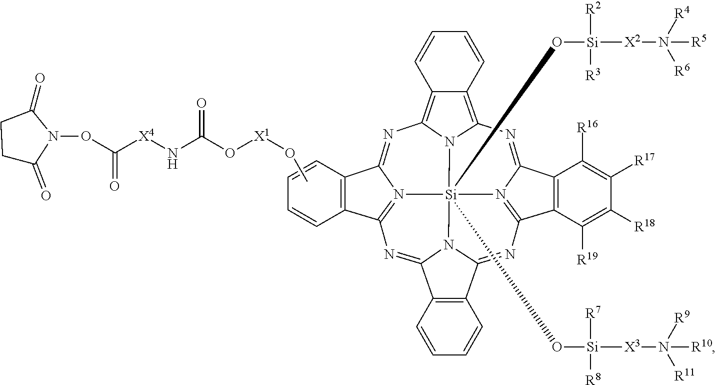

wherein:

X¹ and X⁴ are each independently a C₁-C₁₀ alkylene optionally interrupted by a heteroatom;

R², R³, R⁷, and R⁸ are each independently selected from optionally substituted alkyl and optionally substituted aryl;

R⁴, R⁵, R⁶, R⁹, R¹⁰, and R¹¹ are each independently selected from hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group; and

R¹⁶, R¹⁷, R¹⁸ and R¹⁹ are each independently selected from hydrogen, halogen, optionally substituted alkylthio, optionally substituted alkylamino and optionally substituted alkoxy.

45. The dual conjugate of any of embodiments 1-44, wherein the phthalocyanine dye comprises IRDye 700DX (IR700).

46. The dual conjugate of any of embodiments 1-45, wherein the targeting molecule is an antibody or an antigen-binding fragment thereof

47. The dual conjugate of embodiment 46, wherein the antibody is an antigen-binding fragment that is a Fab, single V_{H} domain, a single chain variable fragment (scFv), a multivalent scFv, a bispecific scFv or an scFv-CH₃ dimer.

48. The dual conjugate of any of embodiments 10-47, wherein the lesion is wherein the lesion is premalignant dysplasia, carcinoma in situ, neoplasm, hyperplasia tumor or a tumor that is associated with a cancer.

49. A composition, comprising the dual conjugate of any of embodiments 1-48.

50. The composition of embodiment 49, further comprising a pharmaceutically acceptable excipient.

51. A kit, comprising:

the dual conjugate of any of embodiments 1-48 or the composition of embodiment 49 or embodiment 50; and

optionally instructions for use.

52. A method of treating a lesion in a subject comprising:

a) administering to the subject a therapeutically effective amount of the dual conjugate of any of embodiments 1-48 or the composition of embodiment 49 or embodiment 50 or the kit of embodiment 51; and

b) after administering the conjugate, irradiating the lesion at a wavelengths to induce phototoxic activity of the conjugate.

53. The method of embodiment 52, wherein irradiating of the lesion is carried out at a wavelength of 500 nm to 900 nm, inclusive, at a dose of at least 1 J cm⁻²or 1 J/cm of fiber length.

54. The method of embodiment 52 or embodiment 53, wherein irradiating of the lesion is carried out at wavelength of 600 nm to 850 nm.

55. The method of any of embodiments 52-54, wherein irradiating of the lesion is carried out at a wavelength of 690±50 nm or at a wavelength of or about 690±20 nm.

56. The method of any of embodiments 52-55, wherein irradiating of the lesion is carried out at a dose of from or from about 2 J cm⁻² to about 400 J cm⁻² or from or from about 2 J/cm fiber length to about 500 J/cm fiber length.

57. The method of any of embodiments 52-56, wherein: irradiating of the lesion is carried out at a dose of at least or at least about 2 J cm⁻², 5 J cm⁻², 10 J cm⁻², 25 J cm⁻², 50 J cm⁻², 75 J cm⁻², 100 J cm⁻², 150 J cm², 200 J cm⁻², 300 J cm⁻², 400 J cm⁻², or 500 J cm⁻²; or

irradiating of the lesion is carried out at a dose of at least or at least about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

58. The method of any of embodiments 52-57, wherein the lesion is a tumor or a tumor that is associated with a cancer.

59. The method of embodiment 58, wherein the tumor is a sarcoma or carcinoma.

60. The method of embodiment 58 or embodiment 59, wherein the tumor is a carcinoma that is a squamous cell carcinoma, basal cell carcinoma or adenocarcinoma.

61. The method of any of embodiments 58-60, wherein the tumor is a carcinoma that is a carcinoma of the bladder, pancreas, colon, ovary, lung, breast, stomach, prostate, cervix, esophagus or head and neck.

62. The method of any of embodiments 58-61, wherein the cancer is a cancer located at the head and neck, breast, liver, colon, ovary, prostate, pancreas, brain, cervix, bone, skin, eye, bladder, stomach, esophagus, peritoneum, or lung.

63. The method of any of embodiments 52-62, wherein irradiating of the lesion is carried out between or between about 30 minutes and about 96 hours after administering the method.

64. The method of any of embodiments 52-63, wherein the dual conjugate is administered at a dose from or from about 50 mg/m² to about 5000 mg/m², from about 250 mg/m² to about 2500 mg/m², from about 750 mg/m² to about 1250 mg/m² or from about 100 mg/m² to about 1000 mg/m².

65. The method of any of embodiments 52-64, further comprising administering an additional therapeutic agent or anti-cancer treatment.

66. The method of embodiment 65, wherein the additional anti-cancer treatment comprises radiation therapy.

67. The method of any of embodiments 52-66, wherein the dual conjugate is combined with another therapeutic for the treatment of the lesion, disease, or condition.

68. The method of any of embodiments 52-67, wherein: the lesion targeted comprises neurons and the disease or condition is a neurological disorder, which optionally comprises pain;

the lesion targeted comprises fat cells or adipocytes and the disease or condition comprises excess fat;

the lesion targeted comprises pathogen infected cells and the disease or condition comprises an infection;

the lesion targeted comprises an inflammatory cell and the disease or condition comprises inflammation.

### VI. EXAMPLES

The following examples are included for illustrative purposes only and are not intended to limit the scope of the invention.

### Example 1: Generation of Cetuximab-IRDye 700DX Conjugate

This Example describes a method for preparing exemplary conjugates containing IRDye 700DX (IR700) linked to exemplary targeting molecules, such as antibodies, to produce antibody-IRDye 700DX (antibody-IR700). The provided methods are exemplary and similar methods may be employed to conjugate other targeting molecules, such as other antibodies or non-antibody targeting molecules, to IRDye 700Dx. The methods were performed to limit exposure of the dye and conjugate to light due to the photosensitivity of the dye, which included the use of low levels of green light having a wavelength from 425 to 575 nm and an intensity of less than 200 Lux in the manufacturing facility. The following buffers were used for conjugation: conjugation buffer (100 mM sodium phosphate, pH 8.65), quenching buffer (1.0 M glycine, pH 9) and final phosphate buffered saline (PBS) formulation buffer: (5.60 mM Na₂HPO₄, 1.058 KH₂PO₄, 154 mM NaCl, pH 7.1).

A. Preparation of Dye and Cetuximab

1. Cetuximab Preparation

Prior to conjugation, Cetuximab (Myoderm USA, Norristown, Pa.) was filtered through a 0.22 μm filter, pooled, and stored at 2-8° C.

A concentration and buffer exchange step was then performed by ultrafiltration/diafiltration (UF/DF). The UF/DF device was cleaned and equilibrated with 100 mM sodium phosphate, pH 8.65 buffer. Prior to UF/DF operations, the pooled, filtered Cetuximab was warmed by placing it in an incubator at 25° C. for 120-150 min. The material was first concentrated to a target of 5 mg/mL and then diafiltered into 100 mM sodium phosphate, pH 8.65 buffer. The diafiltered Cetuximab product concentration was determined and then diluted to a target concentration of 2 mg/mL (1.8-2.4 mg/mL) using 100 mM sodium phosphate, pH 8.65 buffer.

2. Dye Preparation

Prior to conjugation, IRDye 700DX NHS Ester (dye; Cat. No. 929-70011; Li-COR, Lincoln, Nebr.) was prepared by dissolving it to a concentration of 10 mg/mL in anhydrous DMSO. The steps were performed under green light (e.g., wavelength from 425 to 575 nm and an intensity of less than 200 Lux) to protect the dye from the wavelengths of light that are strongly absorbed by the dye.

B. Conjugation

The conjugation and quenching steps were performed in carboys containing diafiltered Cetuximab, wrapped in aluminum foil for light protection. The steps were performed at room temperature under green light (e.g., wavelength from 425 to 575 nm and an intensity of less than 200 Lux) to protect the conjugate from photo-degradation.

The conjugation reaction was performed with IRDye 700DX NHS ester in DMSO, at a final molar ratio of 4:1 (IRDye 700DX NHS ester: Cetuximab), to achieve incorporation of approximately 2-3 dye residues per Cetuximab molecule. The IRDye 700DX NHS ester was added to the carboys containing Cetuximab and mixed on a stir plate for 10-15 min. The conjugation reaction then proceeded for 120 min by placing the carboys in a 25° C. incubator.

The conjugation reaction was quenched by the addition of 1 M glycine to a final concentration of 4.2 mM and mixing for 10-12 min. The carboys were incubated for an additional 20-25 min in the 25° C. incubator.

A final UF/DF step was performed to exchange the conjugated product into the final PBS formulation buffer. The quenched conjugate was transferred to the UF/DF system and was first concentrated to 8-10 L followed by diafiltration with 8-12 diavolumes of PBS in order to exchange the product into the final formulation buffer. The protein concentration was determined and if needed, further dilution with PBS was performed to reach a final target product concentration of 2.0 mg/mL (1.8-2.1 mg/mL).

A filtration through a 0.22 μm filter was performed and the Cetuximab-IRDye 700DX conjugate was stored in the dark at 2-8° C. in a 50 L HyQtainer covered with aluminum foil to protect the contents from light. The steps were performed at room temperature under green light to protect the Cetuximab-IRDye 700DX conjugate. The resulting conjugate was submitted for SEC-HPLC analysis to determine concentration, dye to antibody ratio (DAR), identity and purity, and to determine appearance, pH, bioburden, and endotoxin levels.

### Example 2 : Pharmacokinetics and Therapeutic Efficacy of Cetuximab-IRDye 700DX Conjugate

This Example describes the interim results of a clinical study (Phases 1) assessing safety and efficacy in head and neck cancer patients treated with a single or multiple administration of cetuximab-IRDye 700DX conjugate followed, by irradiation to induce photoimmunotherapy (PIT). Pharmacokinetic parameters and tumor response in human patients after single dose administration of cetuximab-IRDye 700DX conjugate were determined to evaluate safety and efficacy of the therapy.

A. Methods

Nine (9) patients with squamous carcinoma of head and neck entered a dose escalation clinical trial. The patients were divided into three (3) dose cohorts, as listed in Table 1 below. Each cohort included three (3) patients. All patients enrolled in the trial had recurrent progressive cancers that had failed multiple rounds of commercially available treatments, some of which had failed previous treatment with the antibody Cetuximab. The study included both patients with HPV positive and negative tumors, and patients with P16 positive and negative tumors.

TABLE 1
Dose Cohorts for Phase I Clinical
Study of Cetuximab-IRDye 700DX

| | No. of | Human Clinical Dose | Human Clinical Dose |
|---|---|---|---|
| Cohort | Patients | (mg/kg) | (mg/m²) |
| 1 | 3 | 4.0 | 160 |
| 2 | 3 | 8.0 | 320 |
| 3 | 3 | 16.0 | 640 |

Intravenous (IV) bags containing the conjugate were prepared from vials containing 50 mL of a 2 mg/mL solution of cetuximab-IRDye 700DX conjugate produced as described in Example 1. As described in Example 1, the vials were packaged in a single carton and then in an opaque pouch prior to use. The handling of cetuximab-IRDye 700DX conjugate and its administration by infusion were performed in a darkened room with less than 400 lux of fluorescent light. No tungsten lighting was ever used during the preparation of the of the infusion bags. Any windows in the room were covered with shades so that the cetuximab-IRDye 700DX conjugate was never directly or indirectly exposed to sunlight.

In a biosafety cabinet or hood with the light switched-off so that the conjugate was exposed to an intensity of light of no more than 200 lux (equivalent to 60 Watt light bulb or 15 Watt fluorescent room light), each vial was removed from the opaque couch and then from the carton. The packaging of each vial containing the conjugate was opened and the contents of that vial were placed into a sterile IV bag until the desired dose of conjugate for infusion was achieved.

The patients were intravenously administered with a single dose of the cetuximab-IRDye 700DX conjugate at the clinical doses set forth above in Table 8A. The conjugate was administered via IV infusion over 2 hours on Day 1. The intravenous (IV) infusion bag was covered during the administration by an opaque sleeve to protect the conjugate from light exposure.

To induce photoimmunotherapy (PIT), one light application with a light having a wavelength of 690 nm was performed at 24 hours ±3 hours (Day 2) post conjugate administration. 690 nm light was administered to the tumor via superficial and interstitial illumination probes. Light treatment was fixed at a low fluence of 50 J/cm² for superficial illumination or 100 J/cm fiber length for interstitial illumination.

For microlens surface light treatment, normal tissue located 0.5-1.0 cm around the periphery of the tumor was also included in the light treatment field to reach microscopic infiltrating disease at the margin of the tumor.

For cylindrical diffuser implantation directly into tumors, standard techniques were used to place brachytherapy catheters, including ultrasound (US) or computerized tomography (CT) guidance based on interventional radiologic methods. In some instances, a brachytherapy grid was employed. Positioning of the catheters was confirmed by lateral X-ray, US or CT. The cylindrical diffuser fibers were then connected to the 690 nm laser console, according to the manufacturer's instructions.

B. Response

Patients with head and neck cancer, treated with a single administration of cetuximab-IRDye 700DX conjugate followed by irradiation to induce photoimmunotherapy (PIT), were assessed for tumor response. The tumor response was evaluated according to the RECIST (Response Evaluation Criteria In Solid Tumors) criteria as outlined in the revised version 1.1 guidelines (RECIST 1.1, see Eisenhauer et al. (2009) European Journal of Cancer, 45:228-247). A response was determined to be a “complete response” (CR) if there was a disappearance of all target lesions, and any pathological lymph nodes (whether target or non-target) were reduced in short axis to <10 mm. A response was determined to be a “partial response” (PR) if there was at least a 30% decrease in the sum of diameter of target lesions (e.g., at least 30% reduction in tumor growth), taking as reference the baseline sum diameters of the target lesions prior to the treatment. The “objective response rate” (ORR) is the percentage of subjects in which a CR or PR response was observed.

### Example 3 : Reduction of Tumor Density by Antibody-IR700 Conjugate-Mediated PIT

Seven (7) patients from the clinical study described in Example 2 above were further assessed for tumor response according to the Choi criteria as measured by a decrease in tumor density, to evaluate efficacy of PIT and to determine the presence of necrosis in PIT-treated tumors.

A. Methods

Seven (7) patients from the clinical study described in Example 2 above were evaluated by computed tomography (CT), prior to treatment, and one (1) month after irradiation to activate PIT. Changes in tumor density, as measured in Hounsfield Units (HU), between the pre-treatment tumor CT scan and the tumor CT scan at one (1) month post-irradiation were determined based on the CT scans.

B. Response

Response to PIT was characterized using the Choi response criteria, as described in Choi et al., (2007) J Clin Oncol. 25:1753-1759. The Choi criteria use changes in tumor density to determine response, and decreased density of tumors on CT is correlated with the development of tumor necrosis. For therapies that cause tumor necrosis without a substantial decrease in one-dimensional tumor size, the Choi criteria can be more predictive of the treatment outcome than the RECIST criteria, which use one-dimensional tumor size (e.g., sum of the longest diameter of target lesions) (see also van der Veldt et al., (2010) Brit J Cancer 102:803-809; Weng et al., (2013) Oncol Letters 6:1707-1712). Representative Choi criteria include the following: (1) Complete Response (CR), defined as disappearance of all target lesions and no new lesions; (2) Partial Response (PR) defined as a decrease in tumor size of ≥10% or decrease in tumor density (Hounsfield unit (HU)) of ≥15% on CT, no new lesions and no obvious progression of nonmeasurable disease; (3) Progressive Disease (PD), defined as an increase of tumor size of ≥10% and does not meet the PR criteria by tumor density (HU) or new lesions or new intratumoral nodules or increase in the size of the existing intratumoral nodules; and (4) Stable or No Response, defined as not qualifying for CR, PR, or PD and no symptomatic deterioration attributed to tumor progression.

The response results are shown in Table 2Error! Reference source not found. The results demonstrated that six (6) patients had tumors that exhibited at least a partial response (PR) under the Choi criteria after PIT treatment with cetuximab-IRDye 700DX conjugate, as indicated by a decrease in tumor density (HU) of ≥15% on CT. These results, taken together with the results described in Example 4 above showing that PIT treatment results in target cell death indicative of necrosis, showed that tumors that have received PIT treatment using cetuximab-IRDye 700DX conjugate and irradiation, exhibit necrosis and substantial reduction in tumor density, as indicated by a response under the Choi criteria. Thus, the results showed that PIT treatment can substantially reduce tumor burden through necrosis and ICD.

TABLE 2
Patient Tumor Density Reduction after
Cetuximab-IR700-mediated PIT.

| Patient | Reduction in tumor density of >15% by CT (HU) |
|---|---|
| 1 | + |
| 2 | + |
| 3 | + |
| 4 | − |
| 5 | + |
| 6 | + |
| 7 | + |

### Example 4 : Immunogenic Cell Death and Immune Activation by Antibody-IR700 Conjugate-Mediated PIT

The following studies were performed to assess whether immune stimulatory changes occur in PIT-treated cells and whether PIT-treated cells have the potential to activate immune cells. To evaluate what immune stimulatory changes occur in PIT-treated cells, cancer cells treated with and without PIT were evaluated for expression of immunogenic cell death (ICD) markers. Immunogenic cell death is a specific type of cell death exhibited by necrotic cells, and is characterized by increased presentation and release of immune stimulatory markers. Cells exhibiting ICD display membrane changes such as elevated surface expression of heat shock protein 90, and secretion of soluble, intracellular markers known as danger associated molecular patterns (DAMPs), such as ATP and high-mobility group-box protein (HMGB1) (Kromer et al. (2013) Annual Review of Immunology, 31:51-72). As shown below, PIT-treated cancer cells exhibit increased HMGB1 secretion when compared to that of the non-PIT treated cells, indicating that the PIT-treated cells exhibit characteristics of necrosis and ICD.

Because the PIT-treated cells exhibited elevated release of HMGB1, follow-up studies were performed to evaluate whether PIT-treated cells could activate immune cells. To determine whether the immune cells could be activated by PIT-treated tumor cells, the PIT and non-PIT treated cancer cells were co-cultured with monocyte derived immature dendritic cells (iDCs). The surface expression of DC maturation/activation markers CD80, CD86, CD40 and MHCII, which get upregulated upon inflammatory stimuli such as immunogenic cell death via PIT, were observed for any changes. Enhancement of co-stimulatory molecules CD80, CD86 and CD40 indicates augmentation in the ability of DCs to activate T cells and increased MHCII represents increased antigen presentation capabilities as DCs mature. Increased expression of both costimulatory molecules and MHCII was seen on iDCs exposed to tumor killed via PIT as compared to control (non-PIT treated tumor cells).

Antigen presenting cell (APC) co-culture was performed using another model system using THP1 cells, a human monocytic cell line that is widely used for in vitro based APC activation and functional assays. Upregulation of activation makers CD86 was seen on THP1 cells that were exposed to PIT killed tumor cells as opposed to THP1 cells which were co-cultured with non PIT treated tumor cells further confirming the immune-stimulatory potential of PIT.

Altogether, the data indicated that PIT-treated cells exhibit markers characteristic of necrosis and ICD, and that the PIT-treated cells have the potential to activate immune cells. Therefore, combination treatment with PIT with an immune-modulating agent may further enhance the immune activating potential of PIT.

A. Estimation of the HMGB1 Levels from Tumor Cells Subjected to PIT via Cetuximab-IR700

A431 and FaDu tumor cell lines were grown in complete RPMI 1640 and complete EMEM media, respectively. The cells were plated at 15,000 cells in 100 μL total volume per well in a 96 well tissue culture plate for adherence overnight. The viability of the cells prior to plating was checked via trypan blue exclusion method and >95% cells were viable.

The next day the cells were treated with cetuximab-IR700 (prepared as described in Example 1) at 500 ng/mL for 1 hr at 37° C. in the CO₂ incubator and then irradiated with 690 nm laser at a light fluence of 32 J/cm². The controls represented wells corresponding to the groups not treated with light.

After undergoing PIT, the media was removed from the treated cells followed by washing of the cells once with PBS. This was followed by addition of serum free version of the media and incubation for 1 hr at 37° C. in the CO₂ incubator. The supernatant was collected post incubation and stored at −20° C. until use.

The culture supernatants from various treated wells were subjected to HMGB1 ELISA (IBL International, cat #ST51011) as per manufacturer's instructions. Briefly, lyophilized HMGB1 control and standard were solvated with diluent buffer according to kit instructions. A calibration standard curve was prepared by diluting HMGB1 standard stock 1:4 in diluent buffer, then serial diluted 1:2 for a total of 6 points (80 ng/mL-2.5 ng/mL). 100 μL/well of diluent buffer was added to each used well of the ELISA plate provided in the kit. 10 μL/well of standard, control, or sample was added to each well, the plate was sealed, and incubated overnight at 37° C. After 20-24 hours unbound sample was washed away with provided wash buffer (diluted to 1× with distilled water). Lyophilized enzyme conjugate was solvated with enzyme conjugate diluent according to kit instructions and was added to washed plate at 100 μL/well. The plate was gently tapped to mix and was then sealed and incubated at room temperature for 2 hours. Excess enzyme conjugate was then washed off with 1× wash buffer and a 1:1 mix of colrea A and colrea B solutions added to plate at 100 μL/well and incubated for 30 min at room temperature. The reaction was then stopped by adding 100 μL/well of stop solution and gently tapping the plate to mix. The amount of yellow product was quantified by its absorption at 450 nm. The HMGB1 standard curve was graphed with 4 parameter logistics and the test sample data interpolated into the standard curve to determine HMGB1 concentration in each sample. The data was depicted as the fold increase over respective no light controls.

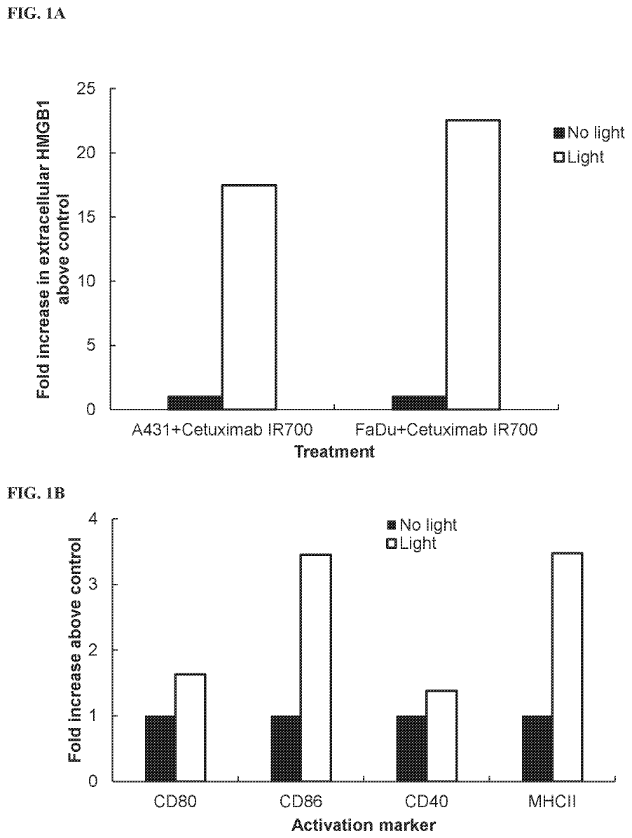

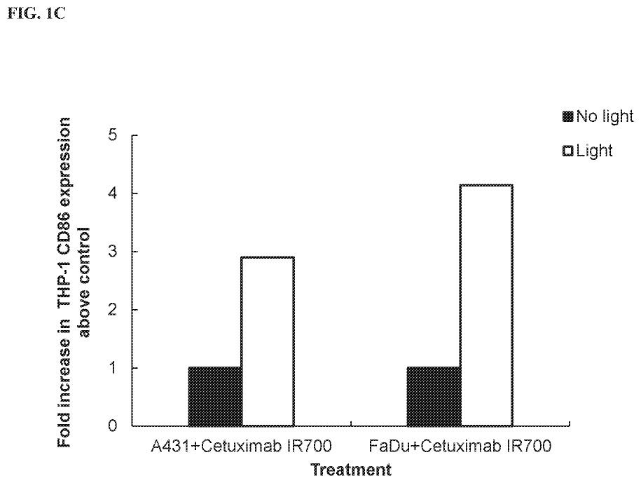

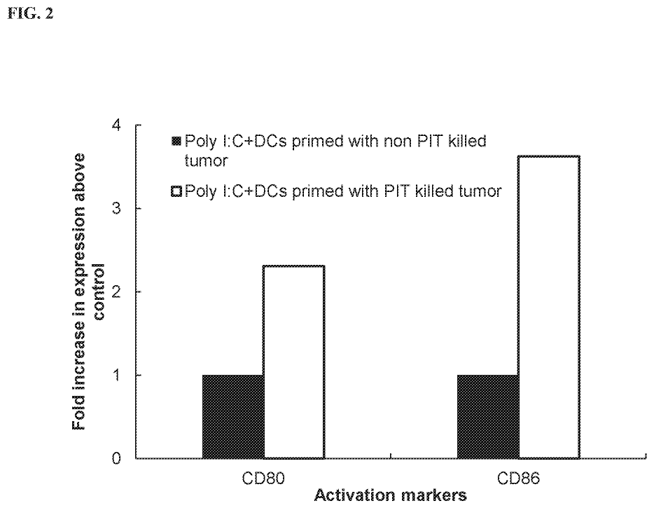

As shown in FIG. 1A, PIT via cetuximab-IR700 resulted in a robust HMGB1 secretion from the tumor cells. Both A431 and FaDu exhibited massive release of HMGB1 as compared to the no light controls. Thus, the results showed that PIT-treatment using cetuximab-IR700 results in cell death that exhibits characteristic of necrosis and ICD.

B. Determination of the Upregulation of DC Maturation Markers CD80, CD86, CD40, and MHCII on DCs Co-Cultured with PIT-Treated Tumor Cells

FaDu cells were grown in complete EMEM media. The cells were plated in 100 μL total volume per well in a 96 well tissue culture plate for adherence overnight. The viability of the cells prior to plating was checked via trypan blue exclusion method and >95% cells were viable.

The next day, the cells were treated with cetuximab-IRDye 700DX at 500 ng/mL for 1 hr at 37° C. in the CO₂ incubator and then were treated with light by subjecting the cells to 690 nm laser light fluence of 12 J/cm². The controls represented wells corresponding to the groups not treated with light (non-PIT treated tumor cells).

For co-culture, human iDCs (Astarte Biologics) from a healthy donor were directly added into the wells with PIT treated tumor cells and control wells (non-PIT treated tumor cells) at 1:1 ratio. The co-cultures were then incubated for 48 hours at 37° C. in the CO₂ incubator. The cells were then detached using a non-enzymatic detachment solution. The harvested cells from various treatment conditions were then incubated with live/dead discrimination dye Zombie Green (BioLegend, 1:500) for 20 min at room temperature followed by washing with stain buffer.

Cells were resuspended in stain buffer and human Fc blocking reagent (BD Biosciences) was then added and cells were incubated for 20 min at room temperature. Anti-human CD80 (BioLegend, clone 2D10), anti-human CD86 (BioLegend, clone IT2.2), anti-human CD40 (BioLegend, clone 5C3), anti-human CD11c (BD, clone B-1y6) and anti-human MHCII (BioLegend, clone L243) antibodies were then added (1:20), cells incubated for 30 min at room temperature. Respective isotype control staining was also performed to assess the background signal. This was followed by a wash and cells resuspended in stain buffer. Data was then acquired via flow cytometry (Attune® Acoustic Focusing Cytometer) under high sensitivity mode. Flow cytometry was performed using anti-human CD14 (clone 63D3, BioLegend, San Diego, Calif.) and anti-human CD86 (clone IT2.2, BioLegend, San Diego, Calif.) antibodies, wherein were added to cells at a 1:40 dilution, and then the cells were incubated for 30 min at room temperature. This was followed by a wash and then the cells were resuspended in stain buffer. Data was then acquired via flow cytometry (Attune® Acoustic Focusing Cytometer, Thermo Fisher Scientific, Waltham, Mass.) under high sensitivity mode. Appropriate gating was done while analyzing the data to exclude cell debris and the data was analyzed with gating performed on live events. The results described below are based on mean fluorescence intensity (MFI) data from each group which is plotted as fold increase over the no light controls.

FIG. 1B shows the upregulation of dendritic cell (DC) maturation markers on iDCs co-cultured with FaDu tumors subjected to PIT via cetuximab-IRDye 700DX. Co-culture with FaDu caused increased surface CD80, CD86, CD40 and MHCII expression on iDCs as compared to the the no light controls. The Y-axis represents fold increase over respective no light controls.

C. CD86 Expression in THP1 Cells Upon Co-Culture with PIT and Non-PIT Treated Tumor Cells

A431 cell line was grown in complete RPMI and T98G, FaDu and U87 tumor cell lines were grown in complete EMEM media. The cells were plated at 15,000 cells in 100 μL total volume per well in a 96 well tissue culture plate for adherence overnight. The viability of the cells prior to plating was checked via trypan blue exclusion method and >95% cells were viable.

The next day the cells were treated with cetuximab-IR700 at 500 ng/mL for 1 hr at 37° C. in the CO₂ incubator and then were treated with light by subjecting the cells to 690 nm laser light fluence of 12 J/cm². The controls represented wells corresponding to the groups not treated with light (non-PIT treated tumor cells).

THP1 cells (ATCC® TIB202™) were grown in complete RPMI. For co-culture, 15,000 THP1 cells were directly added into the wells with PIT treated tumor cells and control non PIT treated tumor cell wells. The co-cultures were then incubated for 24 hours at 37° C. in the CO₂ incubator. On the next day, the cells were then detached using a non-enzymatic detachment solution. The harvested cells from various treatment conditions were then resuspended in PBS only and live/dead discrimination dye Zombie Green (BioLegend) was added (1:500). The cells were incubated for 20 min at room temperature followed by washing with stain buffer.

Cells were resuspended in stain buffer and human Fc blocking reagent (BD Biosciences) was then added and cells were incubated for 20 min at room temperature. Flow cytometry was performed using anti-human CD14 (clone 63D3, BioLegend, San Diego, Calif.) and anti-human CD86 (clone IT2.2, BioLegend, San Diego, Calif.) antibodies, wherein were added to cells at a 1:40 dilution, and then the cells were incubated for 30 min at room temperature. This was followed by a wash and then the cells were resuspended in stain buffer. Data was then acquired via flow cytometry (Attune® Acoustic Focusing Cytometer, Thermo Fisher Scientific, Waltham, Mass.) under high sensitivity mode. Appropriate gating was done while analyzing the data to exclude cell debris and the data was analyzed with gating performed on live events. CD14 marker was used to identify the THP1 cells. The results were based on mean fluorescence intensity (MFI) data from each group which was plotted as fold increase over the no light controls. The data were depicted as fold increase in CD86 surface expression over respective no light controls.

As shown in FIG. 1C, CD86 was upregulated on THP1 cells co-cultured with tumors subjected to PIT via cetuximab-IR700. Co-culture with both A431 and FaDu cells subjected to PIT caused increased surface CD86 expression on THP1 cells as compared to the no light controls.

### Example 5 : PIT in Combination with Treatment with an Immune-Modulator Enhances Immune Activation

Studies were performed to assess whether there is higher immune activation when immune cells are primed with PIT killed tumors and also treated with an immune-modulator. As shown in Example 4, PIT creates an inflammatory environment which leads to activation of immune cells such as dendritic cells (DCs) and monocytes. These PIT primed cells may also exhibit higher potential for further activation when combined with a treatment with an immune-modulator. To test this, PIT-treated tumor cells were co-cultured with monocyte derived immature dendritic cells (iDCs) followed by treatment with the exemplary immune modulatory Poly I:C (a synthetic double stranded RNA analog). Changes in the expression levels of DC activation markers CD80 and CD86 was then assessed. Co-culture of iDCs with non-PIT treated tumor cells was used as controls. Increased CD80 and CD86 expression was seen on DCs that have been previously exposed to an environment where the tumor is killed via PIT versus the condition where the tumor was not treated with PIT.

FaDu cells grown in complete EMEM media were plated in 100 μL total volume per well in a 96 well tissue culture plate for adherence overnight. The viability of the cells prior to plating was checked via the trypan blue exclusion method and >95% cells were found to be viable. The next day the cells were treated with Cetuximab-IRDye 700DX (500 ng/mL for 1 hr at 37° C. in a CO₂ incubator). PIT cell killing was induced by illumination with a 690 nm laser light at a fluence of 12 J/cm². The controls represented wells corresponding to the groups not treated with light.

For co-culture, human iDCs (Astarte Biologics) from a healthy donor were directly added into the wells with PIT killed tumor cells and into control wells (non-PIT treated tumor cells). The co-cultures were then incubated for 48 hours at 37° C. in the CO₂ incubator. The harvested DCs were then subjected to poly I:C treatment (1 μg/mL) for overnight. The cells were then detached using a non-enzymatic detachment solution.

The harvested cells from various treatment conditions were incubated with live/dead discrimination dye Zombie Green (BioLegend, 1:500) for 20 min at room temperature followed by washing with stain buffer. Cells were resuspended in stain buffer and human Fc blocking reagent (BD) was then added and cells were incubated for 20 min at room temperature. Anti-human CD80 (BioLegend, clone 2D10), anti-human CD86 (BioLegend, clone IT2.2), anti-human CD40 (BioLegend, clone 5C3), anti-human CD11c (BD, clone B-1y6) and anti-human MHCII (BioLegend, clone L243) antibodies were added (1:20) and cells were incubated for 30 min at room temperature. Respective isotype control staining was also performed to assess the background signal. Cells were washed and resuspended in stain buffer. Data was then acquired via flow cytometry (Attune® Acoustic Focusing Cytometer) under high sensitivity mode.

Appropriate gating was performed while analyzing the data to exclude cell debris, and the data was analyzed with gating performed on live events. The results described below are based on median fluorescence intensity (MFI) data from each group which is plotted as fold increase over the no light controls.

The results in FIG. 2 showed that dendritic cells (DCs) treated with PIT in combination with an immune-modulator (Poly I:C) exhibited enhanced immune activation as compared to DCs that were not subjected to PIT treatment in combination with an immune modulator. The pre-treatment of DCs with PIT in combination with an immune-modulator leads to increased CD80 and CD86 expression levels compared to the no light (no PIT) controls.

Thus the data indicated that DCs exposed to an environment created by PIT are inherently more predisposed to activation via an immune-modulator. Therefore, combination treatment with PIT with an immune modulating agent may further enhance the immune activating potential of PIT.

### Example 6: Release of Pro-inflammatory Cytokines by Antibody-IR700 Conjugate-Mediated PIT in Combination with Treatment with an Immune-Modulator

Studies were performed to assess whether the enhanced immune activation in immune cells after priming with PIT killed tumors also results in release of pro-inflammatory cytokines/chemokines, and whether the release is further stimulated by an immune modulator.

As shown in Example 4 and Example 5, PIT creates an inflammatory environment which leads to activation of immune cells such as dendritic cells (DCs) and monocytes, and immune modulating agents further enhance the immune activating potential of PIT. Pro-inflammatory cytokines and chemokines released from PIT-primed cells could result in a pro-inflammatory environment near the tumor and regulate migration or recruitment of additional immune cells. Pro-inflammatory cytokines such as TNFα, GM-CSF, IL-1α, IL-1β and IL-12 are involved in differentiation and activation of immune cells involved in an anti-tumor immune response, such as antigen presenting cells (APCs), TH1 and NK cells. Pro-inflammatory chemokines such as IP-10, IL-8, MIP-1α, and MIP-1β can recruit or regulate the migration of immune cells such as T cells and APCs in the tumor microenvironment.

A. Cytokine and Chemokine Production from DCs Co-Cultured with PIT-Treated Tumor Cells

To test whether enhanced immune activation by PIT results in release of pro-inflammatory cytokines and chemokines, PIT-treated tumor cells were co-cultured with monocyte derived immature dendritic cells (iDCs), with or without further stimulation with the exemplary immune modulator Poly I:C. FaDu tumor cells grown in complete EMEM media were plated in 100 μL total volume per well in a 96 well tissue culture plate for adherence overnight. The viability of the cells prior to plating was checked via the trypan blue exclusion method and >95% cells were found to be viable. The next day the cells were treated with an antibody-phthalocyanine dye conjugate (Cetuximab-IRDye 700DX; 500 ng/mL) for 1 hr at 37° C. in a CO₂ incubator. PIT cell killing was induced by illumination with a 690 nm laser light at a fluence of 12 J/cm².

For co-culture, human iDCs (Astarte Biologics) from two healthy donors were directly added into the wells with PIT killed tumor cells and into control wells. Negative control included co-culture of iDCs with untreated tumor cells, iDCs with tumor cells only receiving irradiation, iDCs with tumor cells incubated with Cetuximab-IRDye 700DX without irradiation and iDC only culture. To test whether the iDCs used in the experiment were capable of producing inflammatory cytokines, iDCs incubated with lipopolysaccharide (LPS) were used as positive controls. LPS was added (5 μg/ml) in the last 24 hours of culture to stimulate the iDCs.

The co-cultures were incubated for 48 hours at 37° C. in the CO₂ incubator. The cultured supernantants from various culture conditions were then collected, transferred into Eppendorf tubes, centrifuged for 3 min at 6000 rpm to remove the cells/debris and stored at −80° C. until cytokine/chemokine measurements for selected cytokines/chemokines TNFα, GM-CSF, IL-1α, IL-1β, IL-12, IP-10, IL-8, MIP-1α and MIP-1β.

The culture supernatant samples were subjected to Luminex immunoassay analysis (eBiosciences; Thermo Fisher Scientific) to determine the cytokine and chemokine levels. The samples were run in triplicate, both as undiluted and at 1:5 dilution, to ensure the values were within the detectable range of the procedure. For the negative control and determination of background levels, culture media alone was also subject to the same analysis, and exhibited values lower than the detection limit of the assay for all cytokines and chemokines assessed.

The results of initial cytokine and chemokine analysis are shown in Table 3 and Table 4. Increased levels of pro-inflammatory cytokines were observed in iDCs that were exposed to an environment where the tumor is killed via PIT compared to the negative control environments. Donor derived DCs primed with PIT killed tumors exhibited a consistent (both donors) and robust release of the assessed pro-inflammatory cytokines and chemokines. Taken together with the results in Example 4, showing upregulation of activation markers such as MHCII and CD86 in DCs primed with PIT killed tumors, the results showed that the PIT treatment of tumors can create an immune activating and pro-inflammatory environment.

TABLE 3
Cytokine and Chemokine Production from DC Co-culture Supernatant (Donor 1)

| Culture conditions | TNFα | IL-1β | IP-10 | MIP-1α | MIP-1β | IL-8^{#} |
|---|---|---|---|---|---|---|
| (Donor-1) | (pg/ml) | (pg/ml) | (pg/ml) | (pg/ml) | (pg/ml) | (pg/ml) |
| Tumor + DC (PIT) | 45.5 | 6.9 | 190.8 | 71.3 | 748.7 | 27547.3 |
| | (7.0) | (1.5) | (82.1) | (24.4) | (272) | (154.1) |
| Tumor + DC (irradiation only) | 8.9 | *1.3 | 47.1 | 6.4 | 25.9 | 3925 |
| | (0.2) | (0.8) | (5.2) | (3.6) | (9.7) | (263.8) |
| Tumor + DC (Untreated) | 7.8 | *1.2 | 42.4 | 4.9 | 21 | 3385.6 |
| | (0.2) | (1.7) | (25.7) | (6.7) | (10.8) | (360.3) |
| Tumor + DC | 10.2 | *1.2 | 69.3 | 8.7 | 37.7 | 3286.8 |
| (Cetuximab-IR700 only) | (3.1) | (1.7) | (29.1) | (12) | (20.1) | (235.5) |
| DC only | 43.3 | *0.9 | 20.3 | 35.8 | 297.5 | 5705.7 |
| | (1) | (1.5) | (11.1) | (10.6) | (315) | (150) |
| DC + LPS | 3309.4 | 19.2 | 1574.5 | OOR> | OOR> | 32146.3 |
| | (102.4) | (7.7) | (167) | | | (168.8) |

TABLE 4
Cytokine and Chemokine Production from DC Co-culture Supernatant (Donor 2)

| Culture conditions | TNFα | IL-1β | IP-10 | MIP-1α | MIP-1β | IL-8^{#} |
|---|---|---|---|---|---|---|
| (Donor-2) | (pg/ml) | (pg/ml) | (pg/ml) | (pg/ml) | (pg/ml) | (pg/ml) |
| Tumor + DC (PIT) | 53.1 | 6.5 | 1024.2 | 242.8 | 1565.9 | 25620.5 |
| | (0.7) | (1.2) | (91.6) | (35) | (143) | (203) |
| Tumor + DC (irradiation only) | 20.7 | 3 | 250.9 | 29.7 | 97.4 | 8554.6 |
| | (0) | (1.2) | (172) | (29.3) | (40.8) | (134.2) |
| Tumor + DC (Untreated) | 21.4 | 4.5 | 206.2 | 35.8 | 110.4 | 9773.3 |
| | (1.8) | (1.8) | (42.5) | (12.6) | (47.1) | (37.7) |
| Tumor + DC | 27.9 | 3.9 | 481.9 | 41.2 | 180.2 | 6750.3 |
| (Cetuximab-IR700 only) | (2.7) | (4) | (105) | (4.73) | (33.6) | (94.4) |
| DC only | 24.4 | *1.4 | 299.5 | 56.3 | 207.1 | 6662.8 |
| | (1.5) | (0.5) | (32.3) | (23.8) | (161) | (198.7) |
| DC + LPS | 3524.1 | 18.37 | 1868 | *2293.8 | OOR> | 25819.6 |
| | (388.4) | (1.6) | (410) | (645) | | (215) |

Values in parentheses represent standard deviation
*values extrapolated beyond standard range
^{#}values are depicted at 1:5 dilution
OOR: out of range (above)

B. Cytokine and Chemokine Production from DCs Co-Cultured with PIT-Treated Tumor Cells with Additional Immune Stimulation

The DCs from Donor 1 were further exposed to the exemplary immune modulatory Poly I:C to test whether the immune activation was further enhanced by an immunemodulator. For one set of iDCs (from Donor 1), the co-culture were further subjected to poly I:C treatment for 24 hrs. The culture supernatants were collected post poly I:C stimulation, centrifuged and stored as described above in Example 5A. Cytokine/chemokines were assessed as described above, except additionally levels of GM-C SF and IL-12 were assessed.

The results of cytokine and chemokine production in DCs derived from Donor 1, after further stimulation with Poly I:C, are shown in Table 5. As compared to the results above in Table 1, DCs primed with PIT killed tumors were further activated by treatment with an immune modulator such as Poly I:C as evident by the substantially higher level of cytokine and chemokine release. The extent of cytokine/chemokine levels also was substantially greater in DCs primed with PIT and stimulated with poly I;C compared to DCs primed only with poly I:C. In addition, GM-CSF and IL-12 also were produced in higher amounts in DCs primed with PIT and stimulated with poly I:C, compared to negative control DCs primed with poly I:C. Thus the data indicated that DCs exposed to an environment created by PIT are inherently more predisposed to activation via an immune-modulator. Therefore, combination treatment with PIT with an immune modulating agent may further enhance the immune activating potential of PIT.

TABLE 5
Cytokine and Chemokine Production from DC Co-culture Supernatant after Poly
I:C Stimulation (Donor 1)

| Culture | TNFα | IL-1β | IL-8^{#} | IP10 | MIP-1α^{#} | MIP-1β^{#} | GM-CSF | IL-12p70 |
|---|---|---|---|---|---|---|---|---|
| conditions | (pg/ml) | (pg/ml) | (pg/ml) | (pg/ml) | (pg/ml) | (pg/ml) | (pg/ml) | (pg/ml) |
| Tumor + DC | 1317.7 | 35.4 | 31464.1 | 5453.4 | 5090.3 | *31030.2 | 61.4 | 273.4 |
| (PIT) + Poly | (36.4) | (7.23) | (241) | (214.3) | (253.5) | (307.1) | (8) | (40) |
| I:C | | | | | | | | |
| Tumor + DC | 95.2 | 6.8 | 10621.3 | 2809.6 | 217.7 | 3746.4 | 38.5 | 8.92 |
| (irradiation | (7.77) | (2.29) | (195) | (281.1) | (40.5) | (138.3) | (3.2) | (0) |
| only) + Poly | | | | | | | | |
| I:C | | | | | | | | |
| Tumor + DC | 93.21 | 7.56 | 10912.1 | 1977.5 | 241.8 | 4499.3 | 40.2 | 7.7 |
| (Untreated) + | (3.51) | (4) | (232) | (201.9) | (44.8) | (315.3) | (8.6) | (3.5) |
| Poly I:C | | | | | | | | |
| Tumor + DC | 181.18 | 7.4 | 11068.2 | 2313.9 | 1508.9 | 11327.3 | 46.2 | 22.6 |
| (Cetuximab- | (21) | (1.53) | (160) | (181.9) | (267.2) | (267.7) | (8.3) | (5.3) |
| IR700 only) + | | | | | | | | |
| Poly I:C | | | | | | | | |

Values in parentheses represent standard deviation
*values extrapolated beyond standard range
^{#}values are depicted at 1:5 dilution
OOR: out of range (above)

C. Cytokine Production from PIT-Treated Tumor Cells

To assess whether PIT-treated tumor cells also secrete cytokines upon PIT treatment, the level of pro-inflammatory cytokine IL-la, was tested in the culture supernatant of PIT-treated tumor. FaDu tumor cells were incubated with Cetuximab-IRDye 700DX and irradiated as described above to induce PIT. The experiments were performed twice and the samples were run in triplicate on undiluted supernatant.

The results are shown in Table 6. The results showed that PIT killed tumor cells produce higher amount of the pro-inflammatory cytokine IL-1α than the untreated tumor cells. The results indicate that PIT can induce pro-inflammatory cytokine secretion from the killed tumor cells, in addition to creating a pro-inflammatory microenvironment by the activation of immune cells.

TABLE 6
IL-1α Release from PIT killed FaDu Tumor cells.

| | Expt. 1 | Expt. 2 |
|---|---|---|
| | IL-1α | IL-1α |
| Culture conditions | (pg/ml) | (pg/ml) |
| PIT killed FaDu cells | 3.2 (0.5) | 9.2 (2.2) |
| *Irradiation only treated FaDu cells | 0.6 (0.5) | 1.8 (1.5) |
| *Untreated FaDu cells | 0.6 (0.5) | 1.3 (0.5) |
| *Cetuximab-IR700 only treated FaDu cells | 0.6 (1.3) | 1.8 (0.5) |

Values in parentheses represent standard deviation
*values extrapolated beyond standard range

In summary, the results showed that DCs exposed to a microenvironment of a PIT-treated tumor are inherently more predisposed to the secretion of pro-inflammatory cytokine and chemokines. This response is further enhanced in the presence of stimulation with an immune modulating agent such as poly I:C. Further, tumor cells killed by PIT can also contribute to the creation of inflammatory environment by secretion of pro-inflammatory cytokines.

### Example 7: Combination Treatment with Interferon Gamma and Anti-PD-L1-IR700 PIT

The following studies were performed to assess whether PIT can be combined with immune modulatory agents—which can also affect cancer cells—to enhance PIT-killing activity.

A. Effect of Interferon Gamma on Cell Death

BxPC3 cells (#CRL-1687, ATCC, Manassas Va.) were seeded in 96 well black, clear-flat bottom dishes at 5000 cells per well, and placed in at 37° C., 5% CO₂ incubator. The following day, the cells were washed once with RPMI 1640 supplemented with 10% FBS and 1% Penicillin/Streptomycin (complete culture media). The cells were then incubated for 18 hours with complete culture media containing different concentrations of recombinant human Interferon Gamma (IFNgamma) (carrier free) (Cat No: 570202, BioLegend, San Diego, Calif.) ranging from 0 ng/mL to 3.75 μg/mL.

After 18 hours, the media containing different concentrations of interferon gamma was replaced with complete culture media containing 1× CellTox Green (Cat No: G8731, Promega, Madison, Wisc.). Wells that did not include any cells were also incubated with 1× CellTox Green reagent diluted in complete culture media to serve as background subtraction wells during fluorescent signal detection. The CellTox Green fluoresence signal was measured at 24.5 hours after light treatment using a fluorescence plate reader. The cells were then lysed with detergent, incubated at 37° C. for 30 minutes, and the CellTox Green fluorescence signal was measured again post lysis. The percent dead cells was calculated by taking the ratio between background (1× CellTox Green in complete culture media without cells) subtracted CellTox Green signal per well prior to and post lysis and multiplying the ratio by 100.

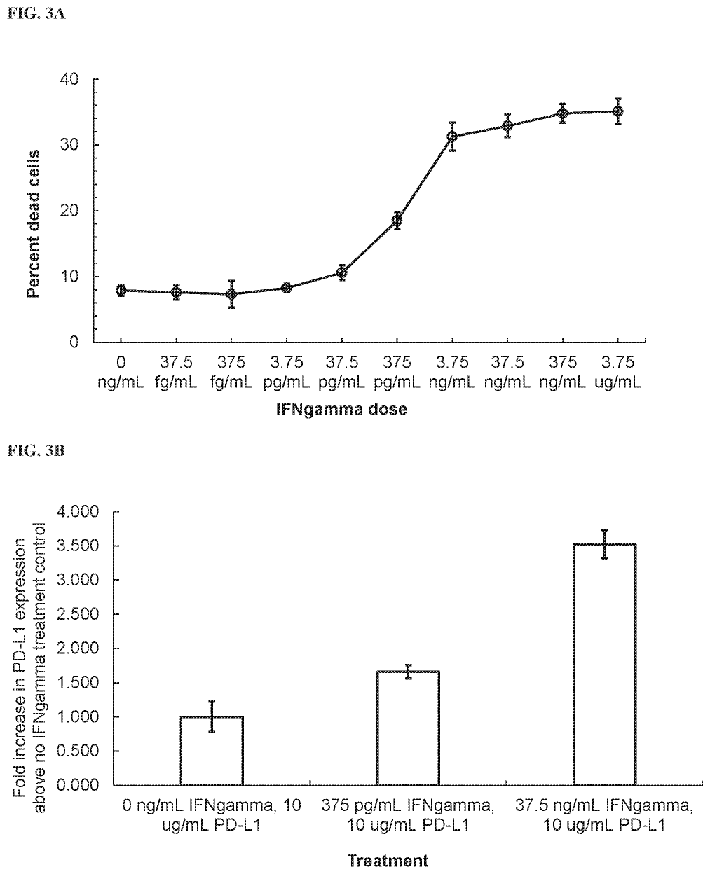

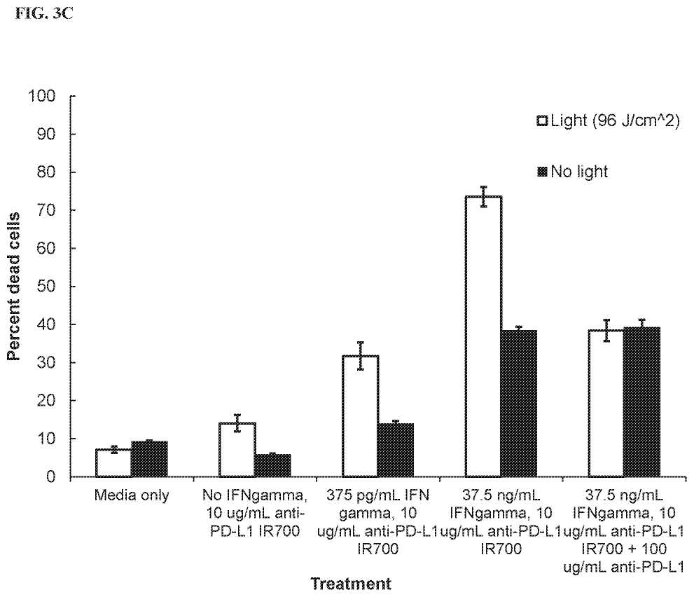

The results in FIG. 3A show the increasing IFNgamma concentration results in a dose-dependent increase in cell death of BxPC3 cells.

B. Effect of Interferon Gamma on PD-L1 Expression

BxPC3 cells were seeded in 12 well dishes at 145,000 cells per well, and placed at 37° C. in a 5% CO₂ incubator. The following day, the cells were washed once with RPMI 1640 supplemented with 10% FBS and 1% Penicillin/Streptomycin (complete culture media). The cells were then incubated for 18 hours with complete culture media alone, complete culture media containing 375 pg/mL of recombinant human Interferon Gamma (carrier free) (Cat No: 570202, BioLegend, San Diego, Calif.), or complete culture media containing 37.5 ng/mL recombinant human Interferon Gamma (carrier free). After the 18 hour incubation with or without recombinant interferon gamma, the BxPC3 cells were washed one time with complete culture media.

The cells were then incubated for one hour at 37° C. with complete culture media alone or complete culture media containing 10 μg/mL anti-PD-L1-IRDye 700DX. The anti-PD-L1-IRDye 700DX was prepared as follows: the antibody solution of mouse anti-human anti-PD-L1 (Catalog No: 329728, Biolegend, San Diego, Calif.) was first exchanged with phosphate buffer saline pH 7 using a 30,000 Dalton molecular weight cutoff centrifugal filter, then the antibody solution pH was adjusted to a pH of 8.5 with addition of phosphate buffer at pH=9. Frozen solid aliquots of IRDye 700DX NHS Ester (Catalog No. 929-70011; Li-COR, Lincoln, Nebr.) were thawed at room temperature, then dissolved with DMSO to achieve a 10 mg/mL concentration. In a dark environment, the solubilized IR700 NHS Ester was then added to the antibody solution at a 4 (IR700 NHS Ester) to 1 (antibody) molar ratio. The conjugation reaction proceeded at 25° C. for 2 hours protected from light. Glycine (pH 8.2) was added to a final concentration of 10 mM for 15 minutes to quench the reaction. The antibody conjugate solution was then exchanged with a 30,000 Dalton molecular weight cutoff centrifugal filter with 24 mL of PBS pH 7 to remove free dye, glycine, and glycine-IR700, and to adjust the pH of the solution back to pH 7.

After the one hour incubation, the cells were washed three times with phosphate buffer saline (pH 7) and incubated with enzyme free cell dissociation buffer (Catalog No: S-014-C, EMD Millipore, Billerica, Mass.) until cells were detached. After the cells detached, phosphate buffer saline containing 0.5% bovine serum albumin fraction V (Catalog No: 15260-037, ThermoFisher Scientific, Waltham, Mass.) was added to the cells, and the samples were immediately analyzed by flow cytometry for PD-L1 expression based on the fluorescent signal from the IR700 dye of the anti-PD-L1-IRDye 700DX. The fold increase in expression was calculated by first subtracting the fluorescent intensity from the anti-PD-L1-IRDye 700DX staining for each treatment from the unstained cells samples, then normalizing each treatment by subtracting the background fluorescent intensity as determined from the mean of the no interferon gamma treated, anti-PD-L1-IRDye 700DX stained samples.

As shown in FIG. 3B, the results showed that increasing IFNgamma concentration resulted in a dose-dependent increase in PD-L1 expression in BxPC3 cells.

C. Combination of Interferon Gamma and Anti-PD-L1-IR700 Conjugate on PIT Cell Killing

Studies were performed to assess if treatment of cells with interferon gamma to increase expression of PD-L1 can enhance anti-PD-L1-mediated PIT killing, BxPC3 cells were seeded in 96 well white, clear-flat bottom dishes at 5000 cells per well, and placed in a 37° C., 5% CO₂ incubator. The following day, the cells were washed once with RPMI 1640 supplemented with 10% FBS and 1% Penicillin/Streptomycin (complete culture media). The cells were then incubated for 18 hours with complete culture media alone, complete culture media containing 375 pg/mL of recombinant human Interferon Gamma (carrier free) (Cat No: 570202, BioLegend, San Diego, Calif.), or complete culture media containing 37.5 ng/mL recombinant human Interferon Gamma (carrier free).

After the 18 hour incubation with or without recombinant interferon gamma, the BxPC3 cells were washed one time with complete culture media. The cells were then incubated for one hour at 37° C. with complete culture media alone or complete culture media containing 10 μg/mL anti-PD-L1-IRDye 700DX or 10 μg/mL anti-PD-L1-IRDye 700DX with 100 ug/mL unconjugated anti-PD-L1. After the one hour incubation, the cells were washed one time complete culture media.

The cells were then illuminated with a 690 nm laser with either 96 J/cm² of light with a 690 nm laser or were protected from light (“no light”). Cell death was assessed using CellTox Green reagent as described above.

As shown in FIG. 3C, combination treatment with IFNgamma prior to treatment with the anti-PD-L1-IR700 conjugate enhanced the anti-PD-L1 photo-activated killing when compared to that of anti-PD-L1-IR700 PIT treatment alone. BxPC3 cells that were not treated with interferon gamma prior to anti-PD-L1-IR700 incubation exhibited a modest increase in cell death upon 690 nm light illumination when compared to that of the no light control. BxPC3 cells incubated with interferon gamma, followed by incubation with anti-PD-L1-IR700 conjugate exhibited an IFNgamma dose dependent increase in basal cell death in the no light treated cells, which is consistent with the effect of IFNgamma to mediate cell death. BxPC3 cells incubated with IFNgamma, incubated with anti-PD-L1-IR700 conjugate, and illuminated with 690 nm light exhibited an IFNgamma dose dependent increase in cell death relative to the no light control for each respective treatment group. The results showed that anti-PD-L1-IR700 PIT killing activity was specific because out-competing anti-PD-L1-IR700 binding with 10× molar excess of unconjugated anti-PD-L1 abrogated the photo-activated killing of the anti-PD-L1-IR700 conjugate as demonstrated by the same percentage of cell death in the light and no light treatments.

The results demonstrated that combination treatment with interferon gamma, an anti-cancer agent and immune modulator, and anti-PD-L1-IR700 PIT exhibits enhanced anticancer activity that of anti-PD-L1-IR700 PIT treatment alone or interferon gamma treatment alone.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID | |
|---|---|
| NO. | Sequence |
| 1 | CRGDKGPDC |
| 2 | CCRGDKGPDC |
| 3 | AKPAPPKPEPKPKKAP |
| 4 | AKVKDEPQRRSARLS |
| 5 | CAGALCY |
| 6 | CAGRRSAYC |
| 7 | CARSKNKDC |
| 8 | CDCRGDCFC |
| 9 | CDTRL |
| 10 | CGKRK |
| 11 | CGLIIQKNEC |
| 12 | CGNKRTR |
| 13 | CGNKRTRGC |
| 14 | CGRRAGGSC |
| 15 | CKAAKNK |
| 16 | CKGGRAKDC-GG |
| 17 | CLSDGKRKC |
| 18 | CMYIEALDKYAC |
| 19 | KKCGGGGIRLRG |
| 20 | CNAGESSKNC |
| 21 | CNGRC |
| 22 | CNRRTKAGC |
| 23 | CPGPEGAGC |
| 24 | CPKTRRPVC |
| 25 | CPRECESIC |
| 26 | CRAKSKVAC |
| 27 | CREAGRKAC |
| 28 | CREKA |
| 29 | CRGDKGPDC |
| 30 | CRGRRST |
| 31 | CRKDKC |
| 32 | CRPPR |
| 33 | CRRETAWAC |
| 34 | CRSRKG |
| 35 | CSRPRRSEC |
| 36 | CTTHWGFTLC |
| 37 | CVPELGHEC |
| 38 | EKGEGALPTGKSK |
| 39 | FALGEA |
| 40 | GLNGLSSADPSSD |
| 41 | GSMSIARL |
| 42 | GVSFLEYR |
| 43 | IFLLWQR |
| 44 | IFLLWQR-C-RR |
| 45 | PEPHC |
| 46 | PISNDQKVSDDDK |
| 47 | RMWPSSTVNLSAGRR |
| 48 | RPARPAR |
| 49 | SMSIARL |
| 50 | VDEDRASLLKSQE |
| 51 | VSFLEYR |
| 52 | WNAPAEEWGNW |
| 53 | PLGLWA |
| 54 | GFLG |

1. A dual conjugate, comprising a phthalocyanine dye, a targeting molecule and a therapeutic agent.

2. The dual conjugate of claim 1, wherein the phthalocyanine dye and therapeutic agent are each independently linked to the targeting molecule.

3. The dual conjugate of claim 1, wherein the targeting molecule and therapeutic agent are each independently linked to the phythalocyanine dye.

4. The dual conjugate of claim 1, wherein the phythalocyanine dye and the targeting molecule are each independently linked to the therapeutic agent.

5. The dual conjugate of claim 1, wherein the dual conjugate comprises the following components:
(phthalocyanine dye)n, (targeting molecule)q and (therapeutic agent)m, wherein:
n, q and m, which are selected independently, are at least 1.

6. The dual conjugate of claim 5, wherein n and q, which are selected independently, are 1 to 5.

7. The dual conjugate of claim 5, wherein n and m, which are selected independently, are 1 to 5.

8. The dual conjugate of claim 5, wherein q is 1, n is between 1 and 100, and m is between 1 and 5.

9. The dual conjugate of claim 5, wherein the ratio of n to q is from or from about 1 to about 1000, from or from about 1 to about 10 or from or from about 2 to about 5.

10. The dual conjugate of any of claims 1-9, wherein the targeting molecule is capable of binding a cell surface molecule on a cell in a microenvironment of a lesion.

11. The dual conjugate of any of claims 1-10, wherein the targeting molecule is linked directly with the phthalocyanine dye or the therapeutic agent.

12. The dual conjugate of any of claims 1-11, wherein the linkage between the targeting molecule and the phthalocyanine dye and/or the therapeutic agent is covalent or non-covalent.

13. The dual conjugate of any of claims 1-10, wherein the phthalocyanine dye is linked directly with the targeting molecule or the therapeutic agent.

14. The dual conjugate of any of claims 1-10 and 13, wherein the linkage between the phthalocyanine dye and the targeting molecule and/or the therapeutic agent is covalent or non-covalent.

15. The dual conjugate of any of claims 1-10, wherein the therapeutic agent is linked directly with the phthalocyanine dye or the targeting molecule.

16. The dual conjugate of any of claims 1-10 and 15, wherein the linkage between the therapeutic agent and the phthalocyanine dye or the targeting molecule is covalent or non-covalent.

17. The dual conjugate of any of claims 1-10, wherein the therapeutic agent is linked indirectly via a linker to the phthalocyanine dye or the targeting molecule.

18. The dual conjugate of any of claims 1-10, wherein the targeting molecule is linked indirectly via a linker to the phthalocyanine dye or the therapeutic agent.

19. The dual conjugate of any of claims 1-10, wherein the phthalocyanine dye is linked indirectly via a linker to the targeting molecule or the therapeutic agent.

20. The dual conjugate of any of claims 17-19, wherein the linker is a peptide or a polypeptide or is a chemical linker.

21. The dual conjugate of any of claims 17-20, wherein the linker is a releasable linker or a cleavable linker.

22. The dual conjugate of claim 21, wherein the releasable linker or the cleavable linker is released or cleaved in the microenvironment of the lesion.

23. The dual conjugate of claim 22, wherein the lesion is a tumor, and the releasable linker or the cleavable linker is released or cleaved in the tumor microenvironment (TME).

24. The dual conjugate of any of claims 21-23, wherein the releasable linker or the cleavable linker is released or cleaved by a matrix metalloproteinase (MMP) present in in the TME.

25. The dual conjugate of any of claims 21-24, wherein the cleavable linker comprises the sequence of amino acids PLGLWA.

26. The dual conjugate of any of claims 21-23, wherein the releasable linker or the cleavable linker is released or cleaved in hypoxic conditions or acidic conditions.

27. The dual conjugate of any of claims 21-23 and 26, wherein the cleavable linker is cleavable under acidic conditions, and the cleavable linker comprises one or more hydrazone, semicarbazone, thiosemicarbazone, cis-aconitic amide, orthoester, acetal, ketal or thioether linkages.

28. The dual conjugate of any of claims 21-23 and 26, wherein the cleavable linker is cleavable under hypoxic conditions, and the linker comprises one or more disulfide linkages.

29. The dual conjugate of any of claims 21-23, wherein the cleavable linker is cleavable by light irradiation, and the linker comprises one or more photolabile phenacyl ester, photolabile hydrazine or photolabile o-nitrobenzyl linkages or photolabile quinoxaline with thioether.

30. The dual conjugate of any of claims 1-29, wherein the therapeutic agent is an immune modulating agent and/or an anti-cancer agent.

31. The dual conjugate of claim 30, wherein the immune modulating agent is a cytokine or is an agent that induces increased expression of a cytokine in the microenvironment of the lesion.

32. The dual conjugate of claim 31, wherein the cytokine is selected from among IL-1, IL-1α, IL-2, IL-3, IL-4, IL-5, IL-6, IL-7, IL-8, IL-9, IL-10, IL-11, IL-12, IL-15, interferon (IFN)-α, IFN-β, IFN-γ, tumor necrosis factor (TNF)-α, TNF-β, human growth hormone, N-methionyl human growth hormone, parathyroid hormone, thyroxine, insulin, proinsulin, relaxin, prorelaxin, glycoprotein hormones such as follicle stimulating hormone (FSH), thyroid stimulating hormone (TSH), and luteinizing hormone (LH), hepatic growth factor, fibroblast growth factor (FGF), prolactin, placental lactogen, tumor necrosis factor-αand -β, mullerian-inhibiting substance, mouse gonadotropin-associated peptide, inhibin, activin, vascular endothelial growth factor (VEGF), integrin, thrombopoietin (TPO), nerve growth factors (NGF)-β, platelet-growth factor, transforming growth factor (TGF)-α, TGF-β, insulin-like growth factor (IGF)-1, IGF-2, erythropoietin (EPO), osteoinductive factors, macrophage-CSF (M-CSF), granulocyte-macrophage-CSF (GM-CSF), granulocyte-CSF (G-CSF), leukemia inhibitory factor (LIF), kit ligand (KL) and/or a portion and/or combination thereof.

33. The dual conjugate of any of claims 30-32, wherein the immune modulating agent is a cytokine and the cytokine is IL-2, IL-4, IL-12, IFN-γ, TNF-α or GM-CSF.

34. The dual conjugate of claim 30, wherein the immune modulating agent is an immune checkpoint inhibitor or an agonist.

35. The dual conjugate of claim 30 or claim 34, wherein the immune modulating agent specifically binds a molecule selected from among CD25, PD-1, PD-L1, PD-L2, CTLA-4, LAG-3, TIM-3, 4-1BB, GITR, CD40, CD40L, OX40, OX40L, CXCR2, B7-H3, B7-H4, BTLA, HVEM, CD28, VISTA, ICOS, ICOS-L, CD27, CD30, STING, and A2A adenosine receptor.

36. The dual conjugate of any of claims 30, 34 and 35, wherein the immune modulating agent is an antibody or an antigen-binding fragment thereof, a small molecule or a polypeptide.

37. The dual conjugate of any of claims 30 and 34-36, wherein the immune modulating agent is selected from among nivolumab, pembrolizumab, pidilizumab, MK-3475, BMS-936559, MPDL3280A, ipilimumab, tremelimumab, IMP31, BMS-986016, urelumab, TRX518, dacetuzumab, lucatumumab, SEQ-CD40, CP-870, CP-893, MED16469, MED14736, MOXR0916, AMP-224, and MSB001078C, or is an antigen-binding fragment thereof.

38. The dual conjugate of claim 30, wherein the anti-cancer agent is an alkylating agent, a platinum drug, an antimetabolite, an anti-tumor antibiotic, a topoisomerase inhibitor, a mitotic inhibitor, a corticosteroid, a proteasome inhibitor, a kinase inhibitor, a histone-deacetylase inhibitor, an anti-neoplastic agent, or a combination thereof.

39. The dual conjugate of claim 30 or claim 38, wherein the anti-cancer agent is an antibody or an antigen-binding fragment thereof, a small molecule or a polypeptide.

40. The dual conjugate of any of claims 30, 38 and 39, wherein the anti-cancer agent is selected from among 5-Fluorouracil/leukovorin, oxaliplatin, irinotecan, regorafenib, ziv-afibercept, capecitabine, cisplatin, paclitaxel, toptecan, carboplatin, gemcitabine, docetaxel, 5-FU, ifosfamide, mitomycin, pemetrexed, vinorelbine, carmustine wager, temozolomide, methotrexate, capacitabine, lapatinib, etoposide, dabrafenib, vemurafenib, liposomal cytarabine, cytarabine, interferon alpha, erlotinib, vincristine, cyclophosphamide, lomusine, procarbazine, sunitinib, somastostatin, doxorubicin, pegylated liposomal encapsulated doxorubicin, epirubicin, eribulin, albumin-bound paclitaxel, ixabepilone, cotrimoxazole, taxane, vinblastine, temsirolimus, temozolomide, bendamustine, oral etoposide, everolimus, octreotide, lanredtide, dacarbazine, mesna, pazopanib, eribulin, imatinib, regorafenib, sorafenib, nilotinib, dasantinib, celecoxib, tamoxifen, toremifene, dactinomycin, sirolimus, crizotinib, certinib, enzalutamide, abiraterone acetate, mitoxantrone, cabazitaxel, fluoropyrimidine, oxaliplatin, leucovorin, afatinib, ceritinib, gefitinib, cabozantinib, oxoliplatin and auroropyrimidine.

41. The dual conjugate of any of claims 30, 38 and 39, wherein the anti-cancer agent is selected from among bevacizumab, cetuximab, panitumumab, ramucirumab, ipilimumab, rituximab, trastuzumab, ado-trastuzumab emtansine, pertuzumab, nivolumab, lapatinib, dabrafenib, vemurafenib, erlotinib, sunitinib, pazopanib, imatinib, regorafenib, sorafenib, nilotinib, dasantinib, celecoxib, crizotinib, certinib, afatinib, axitinib, bevacizumab, bosutinib, cabozantinib, afatinib, gefitinib, temsirolimus, everolimus, sirolimus, ibrutinib, imatinib, lenvatinib, olaparib, palbociclib, ruxolitinib, trametinib, vandetanib or vismodegib, or an antigen-binding fragment thereof.

42. The dual conjugate of any of claims 1-41, wherein the phthalocyanine dye has a maximum absorption wavelength from or from about 600 nm to about 850 nm.

43. The dual conjugate of any of claims 1-42, wherein the phthalocyanine dye comprises the formula: 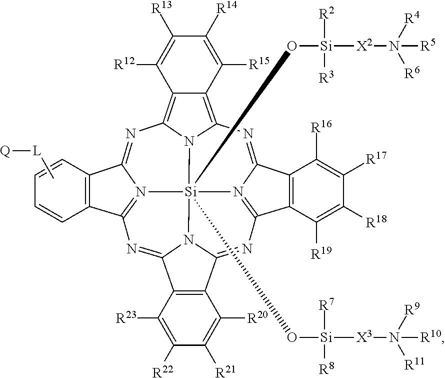
wherein:
L is a linker;
Q is a reactive group for attachment of the dye to the targeting molecule;
R², R³, R⁷, and R⁸ are each independently selected from optionally substituted alkyl and optionally substituted aryl;
R⁴, R⁵, R⁶, R⁹, R¹⁰, and R¹¹ are each independently selected from hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group;
R¹², R¹³, R¹⁴, R¹⁵, R¹⁶, R¹⁷, R¹⁸, R¹⁹, R²⁰, R²¹, R²² and R²³ are each independently selected from hydrogen, halogen, optionally substituted alkylthio, optionally substituted alkylamino and optionally substituted alkoxy; and
X² and X³ are each independently C₁-C₁₀ alkylene, optionally interrupted by a heteroatom.

44. The dual conjugate of any of claims 1-42, wherein the phthalocyanine dye comprises the formula: 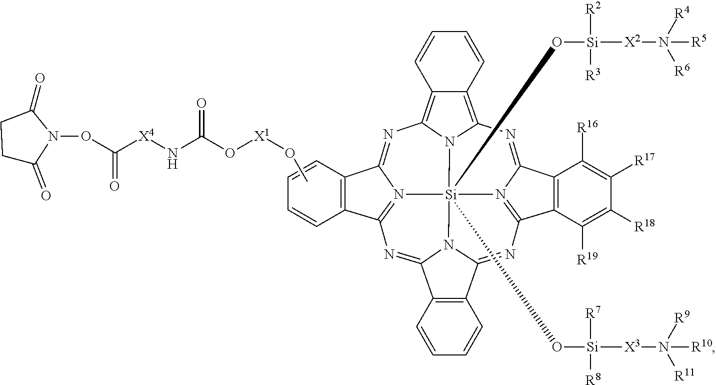
wherein:
X¹ and X⁴ are each independently a C₁-C₁₀ alkylene optionally interrupted by a heteroatom;
R², R³, R⁷, and R⁸ are each independently selected from optionally substituted alkyl and optionally substituted aryl;
R⁴, R⁵, R⁶, R⁹, R¹⁰, and R¹¹ are each independently selected from hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water soluble group; and
R¹⁶, R¹⁷, R¹⁸ and R¹⁹ are each independently selected from hydrogen, halogen, optionally substituted alkylthio, optionally substituted alkylamino and optionally substituted alkoxy.

45. The dual conjugate of any of claims 1-44, wherein the phthalocyanine dye comprises IRDye 700DX (IR700).

46. The dual conjugate of any of claims 1-45, wherein the targeting molecule is an antibody or an antigen-binding fragment thereof.

47. The dual conjugate of claim 46, wherein the antibody is an antigen-binding fragment that is a Fab, single V_{H} domain, a single chain variable fragment (scFv), a multivalent scFv, a bispecific scFv or an scFv-CH₃ dimer.

48. The dual conjugate of any of claims 10-47, wherein the lesion is wherein the lesion is premalignant dysplasia, carcinoma in situ, neoplasm, hyperplasia tumor or a tumor that is associated with a cancer.

49. A composition, comprising the dual conjugate of any of claims 1-48.

50. The composition of claim 49, further comprising a pharmaceutically acceptable excipient.

51. A kit, comprising:
the dual conjugate of any of claims 1-48 or the composition of claim 49 or claim 50; and
optionally instructions for use.

52. A method of treating a lesion in a subject comprising:
a) administering to the subject a therapeutically effective amount of the dual conjugate of any of claims 1-48 or the composition of claim 49 or claim 50 or the kit of claim 51; and
b) after administering the conjugate, irradiating the lesion at a wavelengths to induce phototoxic activity of the conjugate.

53. The method of claim 52, wherein irradiating of the lesion is carried out at a wavelength of 500 nm to 900 nm, inclusive, at a dose of at least 1 J cm⁻²or 1 J/cm of fiber length.

54. The method of claim 52 or claim 53, wherein irradiating of the lesion is carried out at wavelength of 600 nm to 850 nm.

55. The method of any of claims 52-54, wherein irradiating of the lesion is carried out at a wavelength of 690±50 nm or at a wavelength of or about 690±20 nm.

56. The method of any of claims 52-55, wherein irradiating of the lesion is carried out at a dose of from or from about 2 J cm⁻² to about 400 J cm⁻² or from or from about 2 J/cm fiber length to about 500 J/cm fiber length.

57. The method of any of claims 52-56, wherein:
irradiating of the lesion is carried out at a dose of at least or at least about 2 J cm⁻², 5 J cm⁻², 10 J cm⁻², 25 J cm⁻², 50 J cm⁻², 75 J cm⁻², 100 J cm⁻², 150 J cm⁻², 200 J cm⁻², 300 J cm⁻², 400 J cm⁻², or 500 J cm⁻²; or
irradiating of the lesion is carried out at a dose of at least or at least about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

58. The method of any of claims 52-57, wherein the lesion is a tumor or a tumor that is associated with a cancer.

59. The method of claim 58, wherein the tumor is a sarcoma or carcinoma.

60. The method of claim 58 or claim 59, wherein the tumor is a carcinoma that is a squamous cell carcinoma, basal cell carcinoma or adenocarcinoma.

61. The method of any of claims 58-60, wherein the tumor is a carcinoma that is a carcinoma of the bladder, pancreas, colon, ovary, lung, breast, stomach, prostate, cervix, esophagus or head and neck.

62. The method of any of claims 58-61, wherein the cancer is a cancer located at the head and neck, breast, liver, colon, ovary, prostate, pancreas, brain, cervix, bone, skin, eye, bladder, stomach, esophagus, peritoneum, or lung.

63. The method of any of claims 52-62, wherein irradiating of the lesion is carried out between or between about 30 minutes and about 96 hours after administering the method.

64. The method of any of claims 52-63, wherein the dual conjugate is administered at a dose from or from about 50 mg/m² to about 5000 mg/m², from about 250 mg/m² to about 2500 mg/m², from about 750 mg/m² to about 1250 mg/m² or from about 100 mg/m² to about 1000 mg/m².

65. The method of any of claims 52-64, further comprising administering an additional therapeutic agent or anti-cancer treatment.

66. The method of claim 65, wherein the additional anti-cancer treatment comprises radiation therapy.

67. The method of any of claims 52-66, wherein the dual conjugate is combined with another therapeutic for the treatment of the lesion, disease, or condition.

68. The method of any of claims 52-67, wherein:
the lesion targeted comprises neurons and the disease or condition is a neurological disorder, which optionally comprises pain;
the lesion targeted comprises fat cells or adipocytes and the disease or condition comprises excess fat;
the lesion targeted comprises pathogen infected cells and the disease or condition comprises an infection;
the lesion targeted comprises an inflammatory cell and the disease or condition comprises inflammation.
